SEQUENCE LISTING (1 sequence)

SEQ ID NO: 1 - protein, 4 aa, Artificial Sequence
  Description of Artificial Sequence Synthetic peptide
REDV

(19) United States
(12) Patent Application Publication
(10) Pub. No.: US 2022/0213438 A1
(43) Pub. Date: Jul. 7, 2022

(54) NUTRIENT COMPOSITIONS AND METHODS, KITS, AND CELL COMPOSITIONS FOR PRODUCING THE SAME

(71) Applicant: TurtleTree Labs Pte. Ltd., Singapore (SG)

(72) Inventors: Fengru Lin, Singapore (SG); Max Rye, Cathedral City, CA (US); Rabail Hassan Toor, Lahore (PK); Gen Yong, Singapore (SG); Saher Shahid, Lahore (PK)

(21) Appl. No.: 17/703,782

(22) Filed: Mar. 24, 2022

Related U.S. Application Data
(63) Continuation of application No. PCT/US2020/053866, filed on Oct. 1, 2020.
(60) Provisional application No. 63/045,677, filed on Jun. 29, 2020.

(30) Foreign Application Priority Data
Oct. 3, 2019 (SG) 10201909295W

(51) Int. Cl. C12N 5/071; C12N 15/90; C12P 1/00; A23C 9/20; A23J 1/20

(52) U.S. Cl. CPC C12N 5/0631; C12N 15/907; C12P 1/00; C12N 2506/1346; A23J 1/202; A23J 1/205; A23C 9/20

(57) ABSTRACT

Compositions, kits, and methods for preparing nutrient compositions are provided. A nutrient composition can be a food composition such as a dairy composition. A nutrient composition may be produced by an engineered cell composition. Compositions, kits, and methods for preparing engineered cell compositions for producing nutrient compositions are also provided. An engineered cell composition may comprise genetically engineered cells, such as genetically engineered mammary or mammary-like cells. An engineered cell composition can be derived from mammalian stem cells, such as non-mammary adult stem cells. Related methods of characterization, such as for characterizing the engineered cell compositions or the nutrient compositions, are also provided.

### CROSS-REFERENCE

This application is a continuation of International Application No. PCT/US2020/053866, filed Oct. 1, 2020, which claims the benefit of U.S. Provisional Application No. 63/045,677, filed Jun. 29, 2020, and Singapore Patent Application No. 10201909295 W, filed Oct. 3, 2019, each of which applications is incorporated herein by reference in its entirety for all purposes.

### SEQUENCE LISTING

The instant application contains a Sequence Listing which has been submitted in ASCII format via EFS-Web and is hereby incorporated by reference in its entirety. Said ASCII copy, created Apr. 21, 2021, is named 58159_701_301_SL.txt and is 587 bytes in size.

### BACKGROUND

The global population is expected to surpass 9 billion by 2050. Food production may need to substantially increase to fulfill the demand of the growing population, however there are constraints on resources and arable land. Rapidly developing countries such as China, India, and Russia may increase consumption of richer food products, such as meat, dairy, and eggs leading to an increased global demand on these items. Milk is a popular source of nutrition. It comprises high-quality protein, essential minerals (such as calcium, phosphorus, zinc, magnesium), as well as vitamins (such as riboflavin, vitamin A, vitamin B12) among other components. The only dietary component of infant mammals is milk. Milk components also possess advantageous functional characteristics that permit production of a wide variety of derivative dairy products, such as yogurt, cheese, cream, ice cream, and butter, which further contribute to the industrial and cultural significance of milk.

The global dairy market volume is estimated to be 216 metric tons (of which 54% market share is taken up by liquid milk) with the global annual sale of 674 billion USD and world consumption of fresh dairy products and processed dairy products are projected to increase by 2.1% and 1.7% per year respectively over the next decade. Plant-based dairy alternatives further account for $1 billion in the US and an estimated $700 million is estimated for lactose-intolerant milk. According to the report of the Food and Agriculture Organization of the United Nations, greenhouse gas emissions from dairy activities make up a high share of total emissions in some countries and the total livestock sector is responsible for 18% of Greenhouse Gas (GHG) emissions, use 30% of earth's terrain, 70% of arable land, and 8% of global freshwater. In addition, the world's demand for dairy is expected to increase by 22% by 2027, rendering traditional dairy production systems unsustainable. Compared to several dairy sources, particularly bovine milk production, cultured dairy is may decrease energy use, GHG emissions, land use and water use while providing access to dairy products.

### SUMMARY

Cultured dairy products can be an emerging technology in which milk-producing mammalian cells may be produced through in vitro tissue culture in contrast to inefficient traditional livestock agriculture. Compositions and methods are disclosed herein for nutrient compositions (such as liquid nutrient compositions), for example, including at least one lipid, at least one protein, and at least one oligosaccharide. The nutrient composition (such as liquid nutrient composition) can be substantially free of an immunoglobulin, substantially free of whole cells, or substantially free of bacterial microbes. The liquid compositions described herein can be used for consumption by mammals (e.g., humans) as a nutritious composition, a food composition (e.g., a food product), a food supplement, or as a component or ingredient in a food composition.

Multiple cell types may be desirable in creating a cultured dairy product (or dairy composition), as traditional dairy products generally do not solely consist of one cell type or component, but consist of, e.g., mammary epithelial and luminal cells, milk proteins, lipids, and polysaccharides among others. Stem cell differentiation may provide an efficient avenue in producing multiple cell types and components for a heterogeneous cultured dairy product. Forced, transient gene expression in cells such as stem cells and with simultaneous conditioning and expansion in a bioreactor may result in an efficient and holistic approach in developing a cultured dairy product. Provided herein are methods and systems for producing milk and resultant milk products (e.g., in vitro).

In some aspects, the present disclosure provides a liquid composition obtained from an in vitro culture of mammary epithelial cells, the liquid composition comprising, by weight: about 25% to about 90% water, about 0.1% to about 20% of at least one protein, about 0% to about 60% of at least one fat, about 0% to about 30% of at least one carbohydrate, and about 0.0005% to about 3% of at least one mineral, where the liquid composition is supplemented with a nutritionally beneficial amount of a nutrient. In some embodiments, the liquid composition is substantially free of at least one of whole cells, at least one of an antibody, and at least one of a bacterial microbe. In some embodiments, the liquid composition comprises by weight about 0.1% to about 30% lactose. In some embodiments, the mammary epithelial cells are of human origin. In some embodiments, the mammary epithelial cells are of bovine origin. In some embodiments, the bovine cells are dairy cattle cells. In some embodiments, the mammary epithelial cells are of goat origin. In some embodiments, the mammary epithelial cells are of camel origin. In some embodiments, the mammary epithelial cells are of buffalo origin. In some embodiments, the mammary epithelial cells are of sheep origin. In some embodiments, the mammary epithelial cells are of whale origin. In some embodiments, the mammary epithelial cells are of seal origin. In some embodiments, the mammary epithelial cells are of elephant origin. In some embodiments, the mammary epithelial cells are of snow leopard origin. In some embodiments, the liquid composition comprises, by weight, about 40% to about 90% water, about 0.1% to about 15% of at least one protein, about 0% to about 30% of at least one fat, about 0.1% to about 30% of at least one carbohydrate, and about 0.1% to about 3% of at least one mineral. In some embodiments, the liquid composition comprises, by weight, about 40% to about 90% water, about 3% to about 7% of at least one protein, about 3% to about 8% of at least one fat, about 1% to about 5% of at least one carbohydrate, and about 0.1% to about 1% of at least one mineral. In some embodiments, the liquid composition comprises, by weight, about 40% to about 90% water, about 1% to about 2% of at least one protein, about 3% to about 5% of at least one fat, about 7% to about 8% of at least one carbohydrate, and about 0.1% to about 1% of at least one mineral. In some embodiments, the liquid composition further comprising a nutritionally beneficial amount of at least one of Immunoglobulin IgA, lactoferrin, and a probiotic microbe.

In some aspects, the present disclosure provides a food product comprising a composition obtained from an in vitro culture of mammary epithelial cells, the composition comprising, by weight about 25% to about 90% water, about 0.1% to about 20% of at least one protein, about 0% to about 60% of at least one fat, about 0% to about 30% of at least one carbohydrate, and about 0.0005% to about 3% of at least one mineral. In some embodiments, the composition is substantially free of an antibody, substantially free of whole cells, and substantially free of bacterial microbes. In some embodiments, the food product comprises a cheese. In some embodiments, the food product comprises a yogurt. In some embodiments, the food product comprises an infant formula. In some embodiments, the mammary epithelial cells are of human origin. In some embodiments, the composition further comprises a nutritionally beneficial amount of at least one of Immunoglobulin IgA, lactoferrin, and a probiotic microbe. In some embodiments, the composition comprises, by weight about 40% to about 90% water, about 1% to about 2% of at least one protein, about 3% to about 5% of at least one fat, about 7% to about 8% of at least one carbohydrate, and about 0.1% to about 1% of at least one mineral. In some embodiments, the composition further comprises a nutritionally beneficial amount of at least one of Immunoglobulin IgA, lactoferrin, and a probiotic microbe.

In some aspects, the present disclosure provides an engineered cell exhibiting one or more characteristics of a mammary epithelial cell, the engineered cell comprising an attenuated expression mechanism of a target gene or a target transcript as compared to a natural expression mechanism of a corresponding non-engineered cell. In some embodiments, the attenuated expression mechanism comprises an altered promoter sequence of the target gene. In some embodiments, the attenuated expression mechanism effects an altered signaling pathway in the engineered cell. In some embodiments, the attenuated expression mechanism is affected by an endonuclease. In some embodiments, the target gene or the target transcript regulates production of a monosaccharide, a disaccharide, or an oligosaccharide. In some embodiments, the disaccharide is lactose. In some embodiments, the target gene or the target transcript is lactase (LCT). In some embodiments, the target gene or the target transcript is endogenous to the corresponding non-engineered cell.

In some aspects, the present disclosure provides an engineered mammalian cell exhibiting one or more characteristics of a mammary epithelial cell, the engineered cell comprising, in its genome, an exogenous nucleic acid, where the exogenous nucleic acid encodes a hormone or a signaling factor. In some embodiments, the hormone or the signaling factor is selected from a growth factor, glucocorticoid, insulin, progesterone, prolactin, and estrogen. In some embodiments, the exogenous nucleic acid is a heterologous nucleic acid.

In some aspects, the present disclosure provides an engineered cell exhibiting one or more characteristics of a mammary epithelial cell, the engineered cell comprising at least one exogenous nucleic acid, where the at least one exogenous nucleic acid encodes at least one of a glucocorticoid, insulin, transferrin, apo transferrin, progesterone, prolactin, ethanolamine, estrogen, fibroblast growth factor (FGF), FGF receptor, TBX3, NRG3/ERBB4, Wnt/LEF1, insulin-like growth factor 1 (IGF-1), epidermal growth factor (EGF), hepatocyte growth factor (HGF), nuclear factor κB (NF-κB), pTHrP, non-phospho β-catenin, p-p65, RELA, CTNNB1, SMAD3, beclomethasone, betamethasone, budesonide, cortisone, dexamethasone, hydrocortisone, methylprednisolone, prednisolone, prednisone, and triamcinolone. In some embodiments, the exogenous nucleic acid is a heterologous nucleic acid.

In some aspects, the present disclosure provides an engineered cell exhibiting one or more characteristics of a mammary epithelial cell, the engineered cell comprising an inducible gene expression system, the inducible expression system including an exogenous nucleic acid, where the inducible gene expression system is configured to express a hormone or a signaling factor.

In some embodiments, the inducible gene expression system is configured to inducibly express the hormone or the signaling factor. In some embodiments, the hormone or the signaling factor is insulin. In some embodiments, the hormone or the signaling factor is estrogen. In some embodiments, the hormone or the signaling factor is progesterone. In some embodiments, the hormone or the signaling factor is prolactin. Provided herein is an engineered cell exhibiting one or more characteristics of a mammary epithelial cell, the engineered cell comprising an altered genome that effects an enhancement in cellular sensitivity to insulin of the engineered cell as compared to a cellular sensitivity to insulin of a corresponding non-engineered cell. In some embodiments, the altered genome comprises an altered protein tyrosine phosphatase 1B (PTP1B) gene expression mechanism. In some embodiments, the altered genome comprises an altered protein tyrosine phosphatase receptor type F (PTPRF) gene expression mechanism. In some embodiments, the altered genome comprises an altered sirtuin 1 (SIRT1) gene expression mechanism. In some embodiments, the altered SIRT1 gene expression mechanism comprises an altered SIRT1 regulatory pathway that comprises an exogenous inducible promoter. In some embodiments, the altered SIRT1 gene expression mechanism is configured to overexpress SIRT1.

In some aspects, the present disclosure provides a cell culture comprising an effective amount of a reagent, where the reagent is capable of regulating a cellular response to a hormone or a signaling factor of cells in the cell culture. In some embodiments, the reagent is a small molecule, a small interfering ribonucleotide (siRNA), a peptide, a nucleic acid, or a transcription factor. In some embodiments, the reagent is capable of enhancing a cellular response to insulin of the cells in the cell culture. In some embodiments, the reagent is capable of modulating the biological activity of at least one of sirtuin 1 (SIRT1), protein tyrosine phosphatase 1B (PTP1B), and protein tyrosine phosphatase receptor type F (PTPRF) of the cells comprised in the cell culture. In some embodiments, the reagent is capable of enhancing the biological activity of sirtuin 1 (SIRT1) of the cells in the cell culture. In some embodiments, the reagent comprises resveratrol.

In some aspects, the present disclosure provides a liquid composition comprising, by weight, about 25% to about 90% water, about 0.1% to about 20% of at least one protein, about 0% to about 60% of at least one fat, about 0% to about 30% of at least one carbohydrate, and about 0.0005% to about 3% of at least one mineral, and a nutritionally beneficial amount of at least one vitamin, where the liquid composition is substantially free of antibodies, substantially free of bacterial microbes, and substantially free of whole cells. In some embodiments, the liquid composition further comprises a nutritionally beneficial amount of at least one of Immunoglobulin IgA, lactoferrin, and a probiotic microbe.

In some aspects, the present disclosure provides a method for generating an engineered cell, the method comprising: (a) providing an adult stem cell or a derivative thereof; and (b) subjecting the adult stem cell or the derivative thereof to conditions sufficient to generate the engineered cell exhibiting one or more characteristics of a cell of a mammary cell lineage. In some embodiments, the adult stem cell is a non-mammary adult stem cell. In some embodiments, the adult stem cell (or the non-mammary adult stem cell) is a mesenchymal stem cell. In some embodiments, the mammary cell lineage is a mammary epithelial cell lineage. In some embodiments, the mammary epithelial cell lineage is a mammary epithelial luminal cell lineage. In some embodiments, the mammary epithelial cell lineage is a mammary basal cell lineage. In some embodiments, the engineered cell is capable of lactation. In some embodiments, the engineered cell is capable of producing a milk component. In some embodiments, (b) is performed in vitro.

In some aspects, the present disclosure provides a method for generating an engineered cell, the method comprising: (a) providing an adult stem cell or a derivative thereof; and (b) subjecting the adult stem cell or the derivative thereof to conditions sufficient to generate the engineered cell capable of lactation. In some embodiments, the adult stem cell is a non-mammary adult stem cell. In some embodiments, the adult stem cell (or the non-mammary adult stem cell) is a mesenchymal stem cell. In some embodiments, the engineered cell is capable of producing a milk component. In some embodiments, (b) is performed in vitro.

In some embodiments of the method for generating the engineered cell, (b) comprises: contacting the adult stem cell (or the non-mammary adult stem cell) or the derivative thereof with a heterologous polypeptide including a gene regulating moiety, where the gene regulating moiety is configured to regulate an expression of a target gene, a target transcript, or a target protein of the adult stem cell (or the non-mammary adult stem cell) or the derivative thereof, thereby effecting a modified expression profile of the target gene, the target transcript, or the target protein in the engineered cell as compared to a corresponding non-engineered adult stem cell (or a corresponding non-engineered non-mammary adult stem cell). In some embodiments, the target gene, the target transcript, or the target protein is associated with production of a milk component. In some embodiments, the target gene, the target transcript, or the target protein is associated with at least one selected from the group consisting of: CIDEA, XDH, C/EBPβ, HDAC, DGAT1, DGAT2, acyl-CoA:cholesterol acyltransferase, AKT1, S14, LPL, BTN/BTN1A1, ADFP, ACACA/B, FASN, ACLY, S-acyl fatty acid synthase thioesterase, ACOT, SCD, ELOVL, FADS, ACSS, ACSL, GPD1L, GK5, DGKA, GPAM, AGPAT1, LPIN1, LPIN2, SLC27A, FABP, ABCG2, FDFT1, SC4MOL, SC5DL, IDI1, MVD, CH25H, DHCR7, PLIN2, SREBP1, MUC1, MFGE8, mTORC1, CASTOR1, GATOR1, GATOR2, RRAGA/B, RHEB, TSC2, PIK3C3, EAAT3, CAT1, 4F2hc, LAT1, ASCT2, AMPK, STK11, SIRT1, ARNTL, CLOCK, NAMPT, CRY, PER1, CSN1, CSN2, CSN3, LALBA, LTF, LYZ, LPO, TC-1, SPP1, ABL, C3, C4, Galactosyltransferase, B4GALT1, B4GALT2, LGALS1, LGALS2, LGALS3, LGALS8, LGALS9, LGALS12, LGALS13, LGALS14, LGALS16, LCT, GLUT1, FUT1, FUT2, FUT3, FUT4, FUT5, FUT6, FUT7, FUT8, FUT9, sialyltransferase, GALM, GLTP, LGALSL, N-acetylglucosaminyltransferase, glycosyltransferase, and a combination thereof. In some embodiments, the target gene, the target transcript, or the target protein is selected from the group consisting of: CIDEA, XDH, C/EBPβ, HDAC, DGAT1, DGAT2, acyl-CoA:cholesterol acyltransferase, AKT1, S14, LPL, BTN/BTN1A1, ADFP, ACACA/B, FASN, ACLY, S-acyl fatty acid synthase thioesterase, ACOT, SCD, ELOVL, FADS, ACSS, ACSL, GPD1L, GK5, DGKA, GPAM, AGPAT1, LPIN1, LPIN2, SLC27A, FABP, ABCG2, FDFT1, SC4MOL, SC5DL, IDI1, MVD, CH25H, DHCR7, PLIN2, SREBP1, MUC1, MFGE8, mTORC1, CASTOR1, GATOR1, GATOR2, RRAGA/B, RHEB, TSC2, PIK3C3, EAAT3, CAT1, 4F2hc, LAT1, ASCT2, AMPK, STK11, SIRT1, ARNTL, CLOCK, NAMPT, CRY, PER1, CSN1, CSN2, CSN3, LALBA, LTF, LYZ, LPO, TC-1, SPP1, ABL, C3, C4, Galactosyltransferase, B4GALT1, B4GALT2, LGALS1, LGALS2, LGALS3, LGALS8, LGALS9, LGALS12, LGALS13, LGALS14, LGALS16, LCT, GLUT1, FUT1, FUT2, FUT3, FUT4, FUT5, FUT6, FUT7, FUT8, FUT9, sialyltransferase, GALM, GLTP, LGALSL, N-acetylglucosaminyltransferase, glycosyltransferase, and a combination thereof.

In some embodiments of the method for generating the engineered cell, the modified expression profile comprises an up-regulated expression of a gene or a transcript that regulates production of a milk protein. In some embodiments, the modified expression profile comprises a down-regulated expression of a gene or a transcript that regulates production of a milk protein. In some embodiments, the gene or the transcript regulates production of a milk protein selected from the group consisting of: α S1/S2 casein, β casein, κ casein, α-lactalbumin, lactoferrin, lactoperoxidase, lysozyme, haptocorrin, osteopontin, serum albumin, complement C3, complement C4, and a combination thereof.

In some embodiments of the method for generating the engineered cell, the modified expression profile comprises an up-regulated expression of a gene or a transcript that regulates production of a milk carbohydrate. In some embodiments, the modified expression profile comprises a down-regulated expression of gene or a transcript that regulates production of a milk carbohydrate. In some embodiments, the gene or the transcript regulates production of a milk carbohydrate selected from the group consisting of: 2′-fucosyllactose, 3-fucosyllactose, difucosyllactose, difucosyllacto-N-tetrose (DFLNT), difucosyllacto-N-hexaose, fucosyllacto-N-hexaose (FLNH), lacto-N-fucopentaose (LNFP) I, LNFP II, LNFP III, 3′-sialyllactose, 6′-sialyllactose, disialyllacto-N-hexaose (DSLNH), disialyllacto-N-tetraose (DSLNT), fucodisialyllacto-N-hexaose (FDSLNH), sialyl-lacto-N-tetraose b (LSTb), and sialyl-lacto-N-tetraose c (LSTc), lacto-N-hexaose, lacto-N-neotetraose (LNnT), lacto-N-tetrose (LNT), lactose, and a combination thereof. In some embodiments, the gene or the transcript regulating the production of the milk carbohydrate is a gene or a transcript selected from the group consisting of: LALBA, galactosyltransferase B4GALT1, galactosyltransferase B4GALT2, LGALS1, LGALS2, LGALS3, LGALS8, LGALS9, LGALS12, LGALS13, LGALS14, LGALS16, LCT, GLUT1, FUT1, FUT2, FUT3, FUT4, FUT5, FUT6, FUT7, FUT8, FUT9, sialyltransferase, GALM, GLTP, LGALSL, N-acetylglucosaminyltransferase, glycosyltransferase, and a combination thereof.

In some embodiments of the method for generating the engineered cell, the modified expression profile comprises an up-regulated expression of a gene or a transcript that degrades lactose. In some embodiments, the modified expression profile comprises a down-regulated expression of a gene or a transcript that degrades lactose. In some embodiments, the gene or the transcript that degrades the lactose is a gene or a transcript selected from the group consisting of: Lac operon, lactase, and glycosidase.

In some embodiments of the method for generating the engineered cell, the modified expression profile comprises an up-regulated expression of a gene or a transcript that regulates production of a milk fat. In some embodiments, the modified expression profile comprises a down-regulated expression of a gene or a transcript that regulates production of a milk fat. In some embodiments, the gene or the transcript regulating the production of the milk fat is a gene or a transcript selected from the group consisting of: CIDEA, XDH, C/EBPβ, HDAC, DGAT1, DGAT2, acyl-CoA:cholesterol acyltransferase, AKT1, S14, LPL, BTN/BTN1A1, ADFP, ACACA/B, FASN, ACLY, S-acyl fatty acid synthase thioesterase, medium chain/OLAH, ACOT, SCD, ELOVL, FADS, ACSS, ACSL, GDP1L, GK5, DGKA, GPAM, AGPAT1, LPIN1, LPIN2, SLC27A, FABP, ABCG2, FDFT1, SC4MOL, SC5DL, IDI1, MVD, CH25H, DHCR7, PLIN2, SREBP1, MUC1, MFGE8, and a combination thereof.

In some embodiments of the method for generating the engineered cell, the modified expression profile comprises an up-regulated expression of a gene or a transcript that regulates production of a milk lipid. In some embodiments, the modified expression profile comprises a down-regulated expression of a gene or a transcript that regulates production of a milk lipid. In some embodiments, the gene or the transcript regulates production of a milk lipid selected from the group consisting of: palmitic acid, myristic acid, stearic acid, butyric acid, caproic acid, oleic acid, linoleic acid, α-linoleic acid, vaccenic acid, eicosapentaenoic acid, docosahexaenoic acid, calendic acid, γ-linoleic acid, eicosadienoic acid, dihomo-γ-linoleic acid, arachidonic acid, docosadienoic acid, adrenic acid, asbond acid, tetracosatetraenoic acid, tetracospentaenoic acid, hexadecatrienoic acid, stearidonic acid, eicosatrienoic acid, eicosatetraenoic acid, heneicosapentaenoic acid, docosapentaenoic acid, docosahexaenoic acid, tetracosapentaenoic acid, tetracosahexaenoic acid, rumenic acid, caprylic acid, capric acid, and a combination thereof. In some embodiments, the gene or the transcript regulating the production of the milk lipid is a gene or a transcript selected from the group consisting of: CIDEA, XDH, C/EBPβ, HDAC, DGAT1, DGAT2, acyl-CoA:cholesterol acyltransferase, AKT1, S14, LPL, BTN/BTN1A1, ADFP, ACACA/B, FASN, ACLY, S-acyl fatty acid synthase thioesterase, medium chain/OLAH, ACOT, SCD, ELOVL, FADS, ACSS, ACSL, GDP1L, GK5, DGKA, GPAM, AGPAT1, LPINL LPIN2, SLC27A, FABP, ABCG2, FDFT1, SC4MOL, SC5DL, IDI1, MVD, CH25H, DHCR7, PLIN2, SREBP1, MUC1, MFGE8, and a combination thereof.

In some embodiments of the method for generating the engineered cell, the gene or the transcript regulates a cellular response to a hormone or a signaling factor. In some embodiments, the gene or the transcript regulating the cellular response is a gene or a transcript selected from the group consisting of: USF, GATA-1, GATA-2, GATA-3 c-Myc:Max, ATF, USF, CREB, TATA, SREBP-1, Arnt, USF, Tal-1beta, NRF-2, FOXD3, HNF-3beta, v-Myb, FOXJ2, FOXO1, Evi-1, FOXO4, ARP-1, Staf, NF-kappaB, myogenin, AML-1a, Elk-1 Oct-1, Tax/CREB, HNF-1, AP-1, Ahr, Bachl, RP58, AREB6, NKX3A, XFD-1, deltaEF1, poly A downstream element, Pax-4, Sox-5, Sox-9, Zic3, E47, LPL, AGPAT6, CD36, SCD, GPAM, BTN1A1, ACACA, ACSL1, LPIN1, FASN, FABP3, AGPAT6, SREBP1, INSR, PRLR, EGFR, and a combination thereof. In some embodiments, the modified expression profile comprises (1) an up-regulated expression of a first target gene, a first target transcript, or a first target protein, and (2) a down-regulated expression of a second target gene, a second target transcript, or a second target protein. In some embodiments, the modified expression profile is characteristic of a mammary cell lineage. In some embodiments, the modified expression profile comprises a modified expression profile of a gene, a transcript, or a protein selected from the group consisting of: EpCAM, CD14, CD29, CD49b, CD49f, CD61, Sca1, Prominin, ALDEFLUOR, CK14, CK18, and a combination thereof.

In some embodiments of the method for generating the engineered cell, the engineered cell is configured to generate a milk component in response to a hormone or a signaling factor.

In some embodiments of the method for generating the engineered cell, the method further comprises contacting the adult stem cell (or the non-mammary adult stem cell) or the derivative thereof with an exogenous polynucleotide, which exogenous polynucleotide (1) includes an inducible promoter sequence and (2) encodes a hormone or a signaling factor. In some embodiments, the inducible promoter sequence is configured for inducible expression of the hormone or the signaling factor. In some embodiments, the inducible promoter sequence is configured for activation by a reagent. In some embodiments, the engineered cell has an altered response to a hormone or a signaling factor as compared to a corresponding non-engineered adult stem cell (or a corresponding non-engineered non-mammary adult stem cell). In some embodiments, the engineered cell has an enhanced sensitivity to a hormone or a signaling factor as compared to the corresponding non-engineered adult stem cell (or the corresponding non-engineered non-mammary adult stem cell). In some embodiments, the engineered cell has a diminished sensitivity to a hormone or a signaling factor as compared to the corresponding non-engineered adult stem cell (or the corresponding non-engineered non-mammary adult stem cell).

In some embodiments of the method for generating the engineered cell, the hormone or the signaling factor is selected from the group consisting of: glucocorticoid, insulin, transferrin, apo transferrin, progesterone, prolactin, ethanolamine, estrogen, fibroblast growth factor (FGF), FGF receptor, T-box 3 (TBX3), NRG3/ERBB4, Wnt/LEF1, insulin-like growth factor 1 (IGF-1), epidermal growth factor (EGF), hepatocyte growth factor (HGF), nuclear factor κB (NF-κB), pTHrP, non-phospho β-catenin, p-p65, RELA, CTNNB1, SMAD3, and a combination thereof. In some embodiments, the glucocorticoid comprises beclomethasone, betamethasone, budesonide, cortisone, dexamethasone, hydrocortisone, methylprednisolone, prednisolone, prednisone, triamcinolone, or a combination thereof.

In some embodiments of the method for generating the engineered cell, where (b) comprises contacting the adult stem cell (or the non-mammary adult stem cell) or the derivative thereof with a heterologous polypeptide including a gene regulating moiety, the gene regulating moiety is a polynucleotide-guided gene regulating moiety. In some embodiments, the gene regulating moiety is an endonuclease. In some embodiments, the endonuclease is a clustered regularly interspaced short palindromic repeats (CRISPR)-associated (Cas) endonuclease. In some embodiments, the Cas endonuclease is selected from the group consisting of: C2C1, C2C2, C2C3, Cas1, Cas1B, Cas2, Cas3, Cas4, Cas5, Cas5e, Cas6, Cas6e, Cas6f, Cas7, Cas8a, Cas8a1, Cas8a2, Cas8b, Cas8c, Cas9, Cas10, Cas10d, Cas10, Cas10d, Cas11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, a modification thereof, and a combination thereof.

In some embodiments of the method for generating the engineered cell, the mammary cell lineage is a mammary epithelial cell lineage. In some embodiments, the mammary cell lineage is a mammary alveolar cell lineage. In some embodiments, the mammary cell lineage is a mammary myoepithelial cell lineage.

In some embodiments of the method for generating the engineered cell, (b) further comprises contacting the adult stem cell (or the non-mammary adult stem cell) or the derivative thereof with a heterologous polynucleotide configured to bind at least a portion of the target gene or the target transcript. In some embodiments, (b) further comprises forming a complex of the gene regulating moiety and the heterologous polynucleotide. In some embodiments, the complex is a clustered regularly interspaced short palindromic repeats (CRISPR)/CRISPR-associated (Cas) protein complex.

In some embodiments of the method for generating the engineered cell, (b) comprises contacting the adult stem cell (or the non-mammary adult stem cell) or the derivative thereof with a differentiation medium under conditions sufficient to obtain the engineered cell.

In some embodiments of the method for generating the engineered cell, (a) comprises isolating the adult stem cell (or the non-mammary adult stem cell) from a tissue or a bodily secretion of a mammalian subject. In some embodiments, the tissue or the bodily secretion comprises adipose tissue, muscle tissue, cord blood, bone marrow, organ tissue, mammary tissue, extra-embryonic tissue, umbilical cord blood, tendon, periodontal ligament, synovial membrane, trabecular bone, bone marrow, nervous system, skin, periosteum, muscle, peripheral blood, breastmilk, or other body fluid, or a combination thereof. In some embodiments, the tissue or the bodily secretion comprises adipose tissue, mammary tissue, umbilical cord, umbilical cord blood, breastmilk, or a combination thereof. In some embodiments, the subject is a mammal. In some embodiments, the mammal is a human, a non-human primate, a horse, a cow, a dairy cow, a buffalo, a goat, a sheep, a camel, an elephant, a snow leopard, a whale, a seal, a pig, a dog, a mouse, a rat, or a rabbit.

Certain aspects of the present disclosure provide a composition comprising the engineered cell produced by the method for generating an engineered cell as described herein.

In some aspects, the present disclosure provides a composition comprising an engineered cell (or engineered adult stem cell, or engineered non-mammary adult stem cell) exhibiting a modified expression profile that is different than an expression profile of a corresponding non-engineered cell (or non-engineered adult stem cell, or non-engineered non-mammary adult stem cell), where the engineered cell (or engineered adult stem cell, or engineered non-mammary adult stem cell) is configured to be differentiated to an engineered cell exhibiting one or more characteristics of a cell of a mammary cell lineage. In some embodiments, the cell of the mammary cell lineage is a cell of a mammary epithelial cell lineage. In some embodiments, the mammary epithelial cell lineage is a mammary epithelial luminal cell lineage. In some embodiments, the mammary epithelial cell lineage is a mammary basal cell lineage. In some embodiments, the engineered cell is an engineered cell capable of lactation.

In some aspects, the present disclosure provides a composition comprising an engineered cell (or engineered adult stem cell, or engineered non-mammary adult stem cell) exhibiting a modified expression profile that is different than an expression profile of a corresponding non-engineered cell (or non-engineered adult stem cell, or non-engineered non-mammary adult stem cell), where the engineered cell (or engineered adult stem cell, or engineered non-mammary adult stem cell) is configured to be differentiated to an engineered cell capable of lactation.

In some embodiments of the composition comprising the engineered cell, the engineered cell (or engineered adult stem cell, or engineered non-mammary adult stem cell) is configured to produce a milk component. In some embodiments, the engineered cell (or engineered adult stem cell, or engineered non-mammary adult stem cell) is an engineered mesenchymal stem cell. In some embodiments, the corresponding non-engineered cell (or non-engineered adult stem cell, or non-engineered non-mammary adult stem cell) is a corresponding non-engineered mesenchymal stem cell. In some embodiments, the mammary cell lineage is a mammary luminal cell lineage, a mammary epithelial cell lineage, a mammary alveolar cell lineage, or a mammary myoepithelial cell lineage.

In some embodiments of the composition comprising the engineered cell, the engineered cell (or engineered adult stem cell, or engineered non-mammary adult stem cell) or an ancestor cell thereof has been brought in contact with a heterologous polypeptide, which heterologous polypeptide includes a gene regulating moiety. In some embodiments, the engineered cell (or engineered adult stem cell, or engineered non-mammary adult stem cell) comprises a heterologous polypeptide including a gene regulating moiety. In some embodiments, the gene regulating moiety is a polynucleotide-guided gene regulating moiety. In some embodiments, the gene regulating moiety is an endonuclease. In some embodiments, the endonuclease is a clustered regularly interspaced short palindromic repeats (CRISPR)-associated (Cas) endonuclease. In some embodiments, the Cas endonuclease is selected from the group consisting of: C2C1, C2C2, C2C3, Cas1, Cas1B, Cas2, Cas3, Cas4, Cas5, Cas5e, Cas6, Cas6e, Cas6f, Cas7, Cas8a, Cas8a1, Cas8a2, Cas8b, Cas8c, Cas9, Cas10, Cas10d, Cas10, Cas10d, Cas11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, a modification thereof, and a combination thereof. In some embodiments, the engineered cell (or engineered adult stem cell, or engineered non-mammary adult stem cell) or an ancestor cell thereof has been brought in contact with a heterologous polynucleotide configured to form a complex with the gene regulating moiety.

In some embodiments of the composition comprising the engineered cell, the gene regulating moiety is configured to regulate an expression of a target gene, a target transcript, or a target protein of the engineered cell (or engineered adult stem cell, or engineered non-mammary adult stem cell). In some embodiments, the target gene, the target transcript, or the target protein is associated with production of a milk component. In some embodiments, the target gene, the target transcript, or the target protein is associated with at least one selected from the group consisting of: CIDEA, XDH, C/EBPβ, HDAC, DGAT1, DGAT2, acyl-CoA:cholesterol acyltransferase, AKT1, S14, LPL, BTN/BTN1A1, ADFP, ACACA/B, FASN, ACLY, S-acyl fatty acid synthase thioesterase, ACOT, SCD, ELOVL, FADS, ACSS, ACSL, GPD1L, GK5, DGKA, GPAM, AGPAT1, LPIN1, LPIN2, SLC27A, FABP, ABCG2, FDFT1, SC4MOL, SC5DL, IDI1, MVD, CH25H, DHCR7, PLIN2, SREBP1, MUC1, MFGE8, mTORC1, CASTOR1, GATOR1, GATOR2, RRAGA/B, RHEB, TSC2, PIK3C3, EAAT3, CAT1, 4F2hc, LAT1, ASCT2, AMPK, STK11, SIRT1, ARNTL, CLOCK, NAMPT, CRY, PER1, CSN1, CSN2, CSN3, LALBA, LTF, LYZ, LPO, TC-1, SPP1, ABL, C3, C4, Galactosyltransferase, B4GALT1, B4GALT2, LGALS1, LGALS2, LGALS3, LGALS8, LGALS9, LGALS12, LGALS13, LGALS14, LGALS16, LCT, GLUT1, FUT1, FUT2, FUT3, FUT4, FUT5, FUT6, FUT7, FUT8, FUT9, sialyltransferase, GALM, GLTP, LGALSL, N-acetylglucosaminyltransferase, glycosyltransferase, and a combination thereof. In some embodiments, the target gene, the target transcript, or the target protein is selected from the group consisting of: CIDEA, XDH, C/EBPβ, HDAC, DGAT1, DGAT2, acyl-CoA:cholesterol acyltransferase, AKT1, S14, LPL, BTN/BTN1A1, ADFP, ACACA/B, FASN, ACLY, S-acyl fatty acid synthase thioesterase, ACOT, SCD, ELOVL, FADS, ACSS, ACSL, GPD1L, GK5, DGKA, GPAM, AGPAT1, LPIN1, LPIN2, SLC27A, FABP, ABCG2, FDFT1, SC4MOL, SC5DL, IDI1, MVD, CH25H, DHCR7, PLIN2, SREBP1, MUC1, MFGE8, mTORC1, CASTOR1, GATOR1, GATOR2, RRAGA/B, RHEB, TSC2, PIK3C3, EAAT3, CAT1, 4F2hc, LAT1, ASCT2, AMPK, STK11, SIRT1, ARNTL, CLOCK, NAMPT, CRY, PER1, CSN1, CSN2, CSN3, LALBA, LTF, LYZ, LPO, TC-1, SPP1, ABL, C3, C4, Galactosyltransferase, B4GALT1, B4GALT2, LGALS1, LGALS2, LGALS3, LGALS8, LGALS9, LGALS12, LGALS13, LGALS14, LGALS16, LCT, GLUT1, FUT1, FUT2, FUT3, FUT4, FUT5, FUT6, FUT7, FUT8, FUT9, sialyltransferase, GALM, GLTP, LGALSL, N-acetylglucosaminyltransferase, glycosyltransferase, and a combination thereof.

In some embodiments of the composition comprising the engineered cell, the modified expression profile comprises an up-regulated expression of a gene or a transcript that regulates production of a milk protein. In some embodiments, the modified expression profile comprises a down-regulated expression of a gene or a transcript that regulates production of a milk protein. In some embodiments, the gene or the transcript regulating the production of the milk protein selected from the group consisting of: casein and whey protein. In some embodiments, the gene or the transcript regulating the production of the milk protein is a gene or a transcript selected from the group consisting of mTORC1, CASTOR1, GATOR1, GATOR2, RRAGA/B, RHEB, TSC2, PIK3C3, EAAT3, CAT1, 4F2hc, LAT1, ASCT2, AMPK, STK11, SIRT1, ARNTL, CLOCK, NAMPT, CRY, PER1, CSN1, CSN2, CSN3, LALBA, LTF, LYZ, LPO, TC-1, SPP1, ABL, C3, C4, and a combination thereof.

In some embodiments of the composition comprising the engineered cell, the modified expression profile comprises an up-regulated expression of a gene or a transcript that regulates production of a milk carbohydrate. In some embodiments, the modified expression profile comprises a down-regulated expression of a gene or a transcript that regulates production of a milk carbohydrate. In some embodiments, the gene or the transcript regulates production of a milk carbohydrate selected from the group consisting of: 2′-fucosyllactose, 3-fucosyllactose, difucosyllactose, difucosyllacto-N-tetrose (DFLNT), difucosyllacto-N-hexaose, fucosyllacto-N-hexaose (FLNH), lacto-N-fucopentaose (LNFP) I, LNFP II, LNFP III, 3′-sialyllactose, 6′-sialyllactose, disialyllacto-N-hexaose (DSLNH), disialyllacto-N-tetraose (DSLNT), fucodisialyllacto-N-hexaose (FDSLNH), sialyl-lacto-N-tetraose b (LSTb), and sialyl-lacto-N-tetraose c (LSTc), lacto-N-hexaose, lacto-N-neotetraose (LNnT), lacto-N-tetrose (LNT), Lactose, and a combination thereof. In some embodiments, the gene or the transcript regulating the production of the milk carbohydrate is a gene or a transcript selected from the group consisting of: LALBA, galactosyltransferase B4GALT1, galactosyltransferase B4GALT2, LGALS1, LGALS2, LGALS3, LGALS8, LGALS9, LGALS12, LGALS13, LGALS14, LGALS16, LCT, GLUT1, FUT1, FUT2, FUT3, FUT4, FUT5, FUT6, FUT7, FUT8, FUT9, sialyltransferase, GALM, GLTP, LGALSL, N-acetylglucosaminyl transferase, glycosyltransferase, and a combination thereof.

In some embodiments of the composition comprising the engineered cell, the modified expression profile comprises an up-regulated expression of a gene or a transcript that degrades lactose. In some embodiments, the modified expression profile comprises a down-regulated expression of a gene or a transcript that degrades lactose. In some embodiments, the gene or the transcript that degrades lactose is a gene or a transcript selected from the group consisting of: Lac operon, lactase, and glycosidase.

In some embodiments of the composition comprising the engineered cell, the modified expression profile comprises an up-regulated expression of a gene or a transcript that regulates production of a milk fat. In some embodiments, the modified expression profile comprises a down-regulated expression of a gene or a transcript that regulates production of a milk fat. In some embodiments, the gene or the transcript regulates production of a milk fat selected from the group consisting of: palmitic acid, myristic acid, stearic acid, butyric acid, caproic acid, oleic acid, linoleic acid, α-linoleic acid, vaccenic acid, eicosapentaenoic acid, docosahexaenoic acid, calendic acid, γ-linoleic acid, eicosadienoic acid, dihomo-γ-linoleic acid, arachidonic acid, docosadienoic acid, adrenic acid, asbond acid, tetracosatetraenoic acid, tetracospentaenoic acid, hexadecatrienoic acid, stearidonic acid, eicosatrienoic acid, eicosatetraenoic acid, heneicosapentaenoic acid, docosapentaenoic acid, docosahexaenoic acid, tetracosapentaenoic acid, tetracosahexaenoic acid, rumenic acid, caprylic acid, capric acid, and a combination thereof. In some embodiments, the gene or the transcript regulating the production of the milk fat is a gene or a transcript selected from the group consisting of: CIDEA, XDH, C/EBPβ, HDAC, DGAT1, DGAT2, acyl-CoA:cholesterol acyltransferase, AKT1, S14, LPL, BTN/BTN1A1, ADFP, ACACA/B, FASN, ACLY, S-acyl fatty acid synthase thioesterase, medium chain/OLAH, ACOT, SCD, ELOVL, FADS, ACSS, ACSL, GDP1L, GK5, DGKA, GPAM, AGPAT1, LPIN1, LPIN2, SLC27A, FABP, ABCG2, FDFT1, SC4MOL, SC5DL, IDI1, MVD, CH25H, DHCR7, PLIN2, SREBP1, MUC1, MFGE8, and a combination thereof.

In some embodiments of the composition comprising the engineered cell, the gene or the transcript regulates a cellular response to a hormone or a signaling factor. In some embodiments, the gene or the transcript regulating the cellular response is a gene or a transcript selected from the group consisting of: USF, GATA-1, GATA-2, GATA-3 c-Myc:Max, ATF, USF, CREB, TATA, SREBP-1, Arnt, USF, Tal-1beta, NRF-2, FOXD3, HNF-3beta, v-Myb, FOXJ2, FOXO1, Evi-1, FOXO4, ARP-1, Staf, NF-kappaB, myogenin, AML-1a, Elk-1 Oct-1, Tax/CREB, HNF-1, AP-1, Ahr, Bachl, RP58, AREB6, NKX3A, XFD-1, deltaEF1, poly A downstream element, Pax-4, Sox-5, Sox-9, Zic3, E47, LPL, AGPAT6, CD36, SCD, GPAM, BTN1A1, ACACA, ACSL1, LPIN1, FASN, FABP3, AGPAT6, SREBP1, INSR, PRLR, EGFR, and a combination thereof.

In some embodiments of the composition comprising the engineered cell, the modified expression profile comprises (i) an up-regulated expression of a first target gene, a first target transcript, or a first target protein, and (ii) a down-regulated expression of a second target gene, a second target transcript, or a second target protein.

In some embodiments of the composition comprising the engineered cell, the modified expression profile is indicative of the mammary cell lineage. In some embodiments, the engineered cell (or engineered adult stem cell, or engineered non-mammary adult stem cell) exhibits a cellular morphology indicative of the mammary cell lineage. In some embodiments, the mammary cell lineage is a mammary epithelial cell lineage. In some embodiments, the mammary cell lineage is a mammary alveolar cell lineage. In some embodiments, the mammary cell lineage is a mammary myoepithelial cell lineage.

In some embodiments of the composition comprising the engineered cell, the engineered cell (or engineered adult stem cell, or engineered non-mammary adult stem cell) is configured to generate a milk component in response to a hormone or a signaling factor. In some embodiments, the engineered cell (or engineered adult stem cell, or engineered non-mammary adult stem cell) comprises an exogenous polynucleotide that (1) includes an inducible promoter sequence and (2) encodes a hormone or a signaling factor. In some embodiments, the inducible promoter sequence is configured for inducible expression of the hormone or the signaling factor. In some embodiments, the inducible promoter sequence is configured for activation by a reagent. In some embodiments, the engineered cell (or engineered adult stem cell, or engineered non-mammary adult stem cell) has an altered response to a hormone or a signaling factor as compared to a corresponding non-engineered cell (or non-engineered adult stem cell, or non-engineered non-mammary adult stem cell). In some embodiments, the engineered cell (or engineered adult stem cell, or engineered non-mammary adult stem cell) has an enhanced sensitivity to a hormone or a signaling factor as compared to the corresponding non-engineered cell (or non-engineered adult stem cell, or non-engineered non-mammary adult stem cell). In some embodiments, the engineered cell (or engineered adult stem cell, or engineered non-mammary adult stem cell) has a diminished sensitivity to a hormone or a signaling factor as compared to the corresponding non-engineered cell (or non-engineered adult stem cell, or non-engineered non-mammary adult stem cell). In some embodiments, the hormone or the signaling factor is selected from the group consisting of: glucocorticoid, insulin, transferrin, apo transferrin, progesterone, prolactin, ethanolamine, estrogen, fibroblast growth factor (FGF), FGF receptor, TBX3, NRG3/ERBB4, Wnt/LEF1, insulin-like growth factor 1 (IGF-1), epidermal growth factor (EGF), hepatocyte growth factor (HGF), nuclear factor κB (NF-κB), pTHrP, non-phospho β-catenin, p-p65, RELA, CTNNB1, SMAD3, and a combination thereof. In some embodiments, the glucocorticoid comprises beclomethasone, betamethasone, budesonide, cortisone, dexamethasone, hydrocortisone, methylprednisolone, prednisolone, prednisone, triamcinolone, or a combination thereof.

In some embodiments of the composition comprising the engineered cell, the engineered cell (or engineered adult stem cell, or engineered non-mammary adult stem cell) is derived from a non-mammary adult stem cell isolated from a tissue or a bodily secretion of a subject. In some embodiments, the tissue or the bodily secretion comprises adipose tissue, muscle tissue, cord blood, bone marrow, organ tissue, mammary tissue, extra-embryonic tissue, umbilical cord blood, tendon, periodontal ligament, synovial membrane, trabecular bone, bone marrow, nervous system, skin, periosteum, muscle, peripheral blood, breastmilk, or other body fluid, or a combination thereof. In some embodiments, the tissue or the bodily secretion comprises adipose tissue, mammary tissue, umbilical cord, umbilical cord blood, breastmilk, or a combination thereof. In some embodiments, the subject is a mammal. In some embodiments, the mammal is a human, a non-human primate, a horse, a cow, a dairy cow, a buffalo, a goat, a sheep, a camel, an elephant, a snow leopard, a whale, a seal, a pig, a dog, a mouse, a rat, or a rabbit.

In some aspects, the present disclosure also provides a method for generating a milk component, the method comprising: (a) providing a reaction vessel including a cell culture, which cell culture comprises an engineered mammary or mammary-like cell derived from an adult stem cell (or a non-mammary adult stem cell); and (b) subjecting the cell culture to conditions sufficient to produce the milk component in vitro. In some embodiments, the cell culture further comprises a culture medium. In some embodiments, the adult stem cell (or non-mammary adult stem cell) is a mesenchymal stem cell. In some embodiments, the engineered mammary or mammary-like cell is an engineered milk-producing cell. In some embodiments, the engineered mammary or mammary-like cell exhibits one or more characteristics of a mammary cell. In some embodiments, the engineered mammary or mammary-like cell exhibits one or more characteristics of a mammary epithelial cell. In some embodiments, the engineered mammary or mammary-like cell exhibits one or more characteristics of a mammary myoepithelial cell. In some embodiments, the cell culture comprises a two-dimensional cell culture including the engineered mammary or mammary-like cell, a three-dimensional cell culture including the engineered mammary or mammary-like cell, or a high-density cell culture including the engineered mammary or mammary-like cell.

In some embodiments of the method for generating the milk component, the engineered mammary or mammary-like cell comprises a genome that is different from a genome of a naturally occurring milk-producing cell. In some embodiments, the engineered mammary or mammary-like cell has been brought in contact with a heterologous polypeptide, which heterologous polypeptide includes a gene regulating moiety. In some embodiments, the gene regulating moiety is a polynucleotide-guided gene regulating moiety. In some embodiments, the gene regulating moiety is an endonuclease. In some embodiments, the endonuclease is a clustered regularly interspaced short palindromic repeats (CRISPR)-associated (Cas) endonuclease. In some embodiments, the Cas endonuclease is selected from the group consisting of: C2C1, C2C2, C2C3, Cas1, Cas1B, Cas2, Cas3, Cas4, Cas5, Cas5e, Cas6, Cas6e, Cas6f, Cas7, Cas8a, Cas8a1, Cas8a2, Cas8b, Cas8c, Cas9, Cas10, Cas10d, Cas10, Cas10d, Cas11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, a modification thereof, and a combination thereof.

In some embodiments of the method for generating the milk component, (b) comprises using the adult stem cell (or non-mammary adult stem cell) or a derivative thereof to generate the engineered mammary or mammary-like cell or a derivative thereof.

In some embodiments of the method for generating the milk component, (b) comprises: contacting the adult stem cell (or non-mammary adult stem cell) or the derivative thereof with a growth medium under conditions sufficient to produce an expanded cell culture.

In some embodiments of the method for generating the milk component, (b) comprises: contacting the adult stem cell (or non-mammary adult stem cell) or the derivative thereof with a differentiation medium under conditions sufficient to obtain the engineered mammary or mammary-like cell.

In some embodiments of the method for generating the milk component, the method further comprises isolating the adult stem cell (or non-mammary adult stem cell) from a tissue or a bodily secretion of a subject. In some embodiments, the tissue or the bodily secretion comprises adipose tissue, muscle tissue, cord blood, bone marrow, organ tissue, mammary tissue, extra-embryonic tissue, umbilical cord blood, tendon, periodontal ligament, synovial membrane, trabecular bone, bone marrow, nervous system, skin, periosteum, muscle, peripheral blood, breastmilk, or other body fluid, or a combination thereof. In some embodiments, the tissue or the bodily secretion comprises adipose tissue, mammary tissue, umbilical cord, umbilical cord blood, breastmilk, or a combination thereof. In some embodiments, the subject is a mammal. In some embodiments, the mammal is a human, a non-human primate, a horse, a cow, a dairy cow, a buffalo, a goat, a sheep, a camel, an elephant, a snow leopard, a whale, a seal, a pig, a dog, a mouse, a rat, or a rabbit.

In some embodiments of the method for generating the milk component, (b) comprises contacting the engineered mammary or mammary-like cell or a derivative thereof with a lactogenic medium under conditions sufficient to produce the milk component. In some embodiments, the lactogenic medium comprises a substance capable of activating an expression of a hormone or a signaling factor in the engineered mammary or mammary-like cell or the derivative thereof. In some embodiments, the lactogenic medium comprises a substance capable of regulating a cellular response to a hormone or a signaling factor of the engineered milk-producing cell or the derivative thereof.

In some embodiments of the method for generating the milk component, the milk component comprises whey protein, casein, a lipid, an oligosaccharide, or a combination thereof.

In some embodiments of the method for generating the milk component, (b) comprises using the cell culture to generate an aqueous medium comprising one or more particles. In some embodiments, a particle of the one or more particles comprises a fat surrounded by a layer. In some embodiments, the layer comprises a milk lipid or fat. In some embodiments, the layer comprises a milk fat globule membrane (MFGM). In some embodiments, the milk fat globule membrane (MFGM) comprises a milk fat globule membrane (MFGM) protein. In some embodiments, the fat comprises a triglyceride. In some embodiments, the aqueous medium comprises a carbohydrate. In some embodiments, the carbohydrate comprises a monosaccharide, a disaccharide, an oligosaccharide, or a combination thereof. In some embodiments, the oligosaccharide comprises from 3 to 20 saccharide units. In some embodiments, the oligosaccharide comprises a terminal lactose moiety. In some embodiments, the carbohydrate comprises a plurality of oligosaccharides comprising the oligosaccharide. In some embodiments, the milk component not comprise lactose. In some embodiments, the one or more particles are emulsified by the layer and dispersed in the aqueous medium. In some embodiments, the milk component comprises a fat, a protein, or a carbohydrate, or a combination thereof. In some embodiments, the protein comprises casein, whey protein, alpha-lactalbumin, lactoferrin, immunoglobulin IgA, lysozyme, and serum albumin, or a combination thereof.

In some embodiments of the method for generating the milk component, the method further comprises isolating the milk component from the cell culture.

In some embodiments of the method for generating the milk component, the method results in a sterile composition comprising the milk component. In some embodiments, the sterile composition is free of whole cells.

Certain aspects of the present disclosure provides a food composition comprising a milk component, which milk component is produced by a method for generating the milk component as described herein.

In some aspects, the present disclosure further provides a food composition comprising a milk component, where the milk component is produced by one or more engineered mammary or mammary-like cells in a culture medium.

In some aspects, the present disclosure further provides a food composition comprising a milk component, where the milk component is produced by one or more engineered mammary or mammary-like cells derived from a non-mammary adult stem cell. In some embodiments, the milk component is produced by the one or more engineered mammary or mammary-like cells in a culture medium.

In some embodiments of the food composition, the culture medium comprises one or more particles. In some embodiments, a particle of the one or more particles comprises a fat surrounded by a layer. In some embodiments, the layer comprises a milk lipid or fat. In some embodiments, the layer comprises a milk fat globule membrane (MFGM). In some embodiments, the milk fat globule membrane (MFGM) comprises a milk fat globule membrane (MFGM) protein. In some embodiments, the fat of the particle comprises a triglyceride. In some embodiments, the culture medium comprises a carbohydrate. In some embodiments, the carbohydrate comprises a monosaccharide, a disaccharide, an oligosaccharide, or a combination thereof. In some embodiments, the oligosaccharide comprises from 3 to 20 saccharide units. In some embodiments, the oligosaccharide comprises a terminal lactose moiety. In some embodiments, the carbohydrate comprises a plurality of oligosaccharides comprising the oligosaccharide. In some embodiments, the milk component not comprise lactose. In some embodiments, the one or more particles are emulsified by the layer and dispersed in the culture medium.

In some embodiments, the food composition is a dairy composition selected from the group consisting of: milk, yogurt, cheese, cream, and butter. In some embodiments, the milk is selected from colostrum, mature milk, fore milk, hind milk, and transition milk. In some embodiments, the milk is formulated for a subject of an age. In some embodiments, the milk is formulated for a child of an age three-years or younger. In some embodiments, the milk is formulated for an infant of twelve-month old or younger.

In some embodiments of the food composition, the milk component comprises a fat, a protein, a carbohydrate, or a combination thereof. In some embodiments, the protein comprises casein, whey protein, alpha-lactalbumin, lactoferrin, immunoglobulin IgA, lysozyme, serum albumin, or a combination thereof. In some embodiments, the carbohydrate comprises a monosaccharide, a disaccharide, an oligosaccharide, or a combination thereof. In some embodiments, the oligosaccharide comprises from 3 to 20 saccharide units. In some embodiments, the oligosaccharide comprises a terminal lactose moiety. In some embodiments, the carbohydrate comprises a plurality of oligosaccharides. In some embodiments, the carbohydrate does not comprise lactose. In some embodiments, the fat comprises a triglyceride.

In some embodiments, the milk component comprises, by weight, at least (about) 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% protein. In some embodiments, the milk component comprises, by weight, at most (about) 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or 0.5% protein. In some embodiments, the milk component comprises protein, by weight, in a range of any two of the following values: (about) 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

In some embodiments, the milk component comprises, by weight, at least (about) 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% carbohydrate. In some embodiments, the milk component comprises, by weight, at most (about) 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.2%, 0.1%, 0.05%, or 0.01% carbohydrate. In some embodiments, the milk component comprises carbohydrate, by weight, in a range of any two of the following values: (about) 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the milk component is substantially free of carbohydrate. In some embodiments, the milk component does not comprise carbohydrate. In some embodiments, the milk component comprises, by weight, at least (about) 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% lactose. In some embodiments, the milk component comprises, by weight, at most (about) 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.2%, 0.1%, 0.05%, or 0.01% lactose. In some embodiments, the milk component comprises lactose, by weight, in a range of any two of the following values: (about) 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the milk component is substantially free of lactose. In some embodiments, the milk component does not comprise lactose.

In some embodiments, the milk component comprises, by weight, at least (about) 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% fat. In some embodiments, the milk component comprises, by weight, at most (about) 60%, 57%, 55%, 53%, 50%, 47%, 45%, 43%, 40%, 37%, 35%, 33%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.2%, 0.1%, 0.05%, or 0.01% fat. In some embodiments, the milk component comprises fat, by weight, in a range of any two of the following values: (about) 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 33%, 35%, 37%, 40%, 43%, 45%, 47%, 50%, 53%, 55%, 57%, or 60%. In some embodiments, the milk component is substantially free of fat. In some embodiments, the milk component does not comprise fat.

In some embodiments, the milk component comprises, by weight:

(i) from about 1% to about 20%, from about 2% to about 20%, from about 3% to about 20%, from about 4% to about 20%, from about 5% to about 20%, from about 1% to about 15%, from about 2% to about 15%, from about 3% to about 15%, from about 4% to about 15%, from about 5% to about 15%, from about 1% to about 10%, from about 2% to about 10%, from about 3% to about 10%, from about 4% to about 10%, from about 5% to about 10%, from about 1% to about 9%, from about 2% to about 9%, from about 3% to about 9%, from about 4% to about 9%, from about 5% to about 9%, from about 1% to about 8%, from about 2% to about 8%, from about 3% to about 8%, from about 4% to about 8%, from about 5% to about 8%, from about 1% to about 7%, from about 2% to about 7%, from about 3% to about 7%, from about 4% to about 7%, or from about 5% to about 7% fat,

(ii) from about 0.1% to about 20%, from about 0.2% to about 20%, from about 0.3% to about 20%, from about 0.4% to about 20%, from about 0.5% to about 20%, from about 0.6% to about 20%, from about 0.7% to about 20%, from about 0.8% to about 20%, from about 0.9% to about 20%, from about 1% to about 20%, from about 0.1% to about 15%, from about 0.2% to about 15%, from about 0.3% to about 15%, from about 0.4% to about 15%, from about 0.5% to about 15%, from about 0.6% to about 15%, from about 0.7% to about 15%, from about 0.8% to about 15%, from about 0.9% to about 15%, from about 1% to about 15%, from about 0.1% to about 10%, from about 0.2% to about 10%, from about 0.3% to about 10%, from about 0.4% to about 10%, from about 0.5% to about 10%, from about 0.6% to about 10%, from about 0.7% to about 10%, from about 0.8% to about 10%, from about 0.9% to about 10%, from about 1% to about 10%, from about 0.1% to about 8%, from about 0.2% to about 8%, from about 0.3% to about 8%, from about 0.4% to about 8%, from about 0.5% to about 8%, from about 0.6% to about 8%, from about 0.7% to about 8%, from about 0.8% to about 8%, from about 0.9% to about 8%, from about 1% to about 8%, from about 0.1% to about 7%, from about 0.2% to about 7%, from about 0.3% to about 7%, from about 0.4% to about 7%, from about 0.5% to about 7%, from about 0.6% to about 7%, from about 0.7% to about 7%, from about 0.8% to about 7%, from about 0.9% to about 7%, from about 1% to about 7%, from about 0.1% to about 5%, from about 0.2% to about 5%, from about 0.3% to about 5%, from about 0.4% to about 5%, from about 0.5% to about 5%, from about 0.6% to about 5%, from about 0.7% to about 5%, from about 0.8% to about 5%, from about 0.9% to about 5%, from about 1% to about 5% protein, and

(iii) from about 0.1% to about 20%, from about 0.2% to about 20%, from about 0.3% to about 20%, from about 0.4% to about 20%, from about 0.5% to about 20%, from about 0.6% to about 20%, from about 0.7% to about 20%, from about 0.8% to about 20%, from about 0.9% to about 20%, from about 1% to about 20%, from about 0.1% to about 15%, from about 0.2% to about 15%, from about 0.3% to about 15%, from about 0.4% to about 15%, from about 0.5% to about 15%, from about 0.6% to about 15%, from about 0.7% to about 15%, from about 0.8% to about 15%, from about 0.9% to about 15%, from about 1% to about 15%, from about 0.1% to about 10%, from about 0.2% to about 10%, from about 0.3% to about 10%, from about 0.4% to about 10%, from about 0.5% to about 10%, from about 0.6% to about 10%, from about 0.7% to about 10%, from about 0.8% to about 10%, from about 0.9% to about 10%, from about 1% to about 10%, from about 0.1% to about 8%, from about 0.2% to about 8%, from about 0.3% to about 8%, from about 0.4% to about 8%, from about 0.5% to about 8%, from about 0.6% to about 8%, from about 0.7% to about 8%, from about 0.8% to about 8%, from about 0.9% to about 8%, from about 1% to about 8%, from about 0.1% to about 7%, from about 0.2% to about 7%, from about 0.3% to about 7%, from about 0.4% to about 7%, from about 0.5% to about 7%, from about 0.6% to about 7%, from about 0.7% to about 7%, from about 0.8% to about 7%, from about 0.9% to about 7%, from about 1% to about 7%, from about 0.1% to about 5%, from about 0.2% to about 5%, from about 0.3% to about 5%, from about 0.4% to about 5%, from about 0.5% to about 5%, from about 0.6% to about 5%, from about 0.7% to about 5%, from about 0.8% to about 5%, from about 0.9% to about 5%, from about 1% to about 5% carbohydrate.

In some embodiments, the food composition is sterile. In some embodiments, the food composition is lactose-free. In some embodiments, the food composition further comprises a mineral. In some embodiments, the food composition further comprises a vitamin. In some embodiments, the food composition further comprises an amino acid. In some embodiments, the food composition further comprises one or more probiotic bacteria. In some embodiments, the one or more probiotic bacteria comprises one or more strains selected from the group consisting of: Bifidobacterium, Lactobacillus, Clostridium, Ralstonia, Staphylococcus, Streptococcus, and a combination thereof. In some embodiments, the one or more probiotic bacteria comprises one or more strains selected from the group consisting of: Pseudomonas, Staphylococcus, Streptococcus, Elizabethkingia, Variovorax, Bifidobacterium, Flavobacterium, Lactobacillus, Stenotrophomonas, Brevundimonas, Chryseobacterium, Enterobacter, and a combination thereof. In some embodiments, the food composition further comprises an antibody. In some embodiments, the antibody comprises Immunoglobulin IgA. In some embodiments, the food composition further comprises lactoferrin.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a schematic of processes for generating mammary cells or mammary-like cells from isolated stem cells as described herein.

FIG. 2 illustrates a schematic of bioreactor processes for producing milk component(s) from isolated stem cells as described herein.

FIG. 3 illustrates a laboratory workflow for preparing genetically engineered mammary or mammary-like cell(s) as described herein.

FIG. 4 illustrates a schematic of a Tet-On inducible promoter system that can be used to induce expression of casein in mammary or mammary-like cells as described herein.

FIG. 5 illustrates a computer system programmed or otherwise configured to implement method(s) provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least (about),” “greater than (about),” or “greater than or equal to” precedes the first numerical value in a series of two or more numerical values, the term “at least (about),” “greater than (about)” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “at most (about),” “no more than (about),” “less than (about),” or “less than or equal to” precedes the first numerical value in a series of two or more numerical values, the term “at most (about),” “no more than (about),” “less than (about),” or “less than or equal to” applies to each of the numerical values in that series of numerical values. For example, less than or equal to 3, 2, or 1 is equivalent to less than or equal to 3, less than or equal to 2, or less than or equal to 1.

The use of the word “a” or “an,” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” is generally used to indicate that a value includes the inherent variation of error for the device, the method being employed to determine the value, or the variation that exists among the study subjects. Unless otherwise specified based upon the above values, the term “about” may mean±5% of the listed value.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “flavor” generally refers to the taste and/or the aroma of a food or drink.

As used herein, the terms “ash” or “mineral” generally refer to one or more ions, elements, minerals, and/or compounds that can be found in a mammal-produced milk. Non-limiting ions, elements, minerals, and compounds that are found in a mammal-produced milk are described herein. Additional ions, elements, minerals, and compounds may be found in a mammal-produced milk.

As used herein, the term “nucleic acid,” generally refers to a polymeric form of nucleotides of any length (e.g., at least 2, 3, 4, 5, 6, 7, 8, 9, 10, 100, 500, 1000 or more nucleotides), either deoxyribonucleotides or ribonucleotides, or analogs thereof. A nucleic acid may include one or more subunits selected from adenosine (A), cytosine (C), guanine (G), thymine (TO, and uracil (U), or variants thereof. A nucleotide can include A, C, G, T, or U, or variants thereof. A nucleotide can include any subunit that can be incorporated into a growing nucleic acid strand. Such subunit can be A, C, G, T, or U, or any other subunit that is specific to one of more complementary A, C, G, T, or U, or complementary to a purine (e.g., A or G, or variant thereof) or pyrimidine (e.g., C, T, or U, or variant thereof). In some examples, a nucleic acid may be single-stranded or double stranded, in some cases, a nucleic acid molecule is circular. Non-limiting examples of nucleic acids include deoxyribonucleic acid (DNA) and ribonucleic acid (RNA). Nucleic acids can include coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid molecule may comprise one or more modified nucleotides, such as methylated nucleotides and nucleotide analogs.

As used herein, the term “lipids” generally refers to one or more molecules (e.g., biomolecules) that include a fatty acyl group (e.g., saturated or unsaturated acyl chains). A lipid may include oils, phospholipids, free fatty acids, phospholipids, monoglycerides, diglycerides, and triglycerides.

As used herein, an “isolated” organic molecule (e.g., a fatty acid or a SCFA) generally refers to one which is substantially separated from the cellular components (membrane lipids, chromosomes, proteins) of the host cell from which it originated. As used herein, the term “isolated” with respect to protein generally indicates that the preparation of protein is at least 60% pure, e.g., greater than 60%, 65%, 70%, 75%, 80%, 85%, 90%, 95%, or 99% pure. The term does not require that the biomolecule has been separated from all other chemicals, although certain isolated biomolecules may be purified to near homogeneity.

As used herein, the term “micelle” generally refers to a spherical or a roughly spherical supramolecular structure that exists as a dispersion within a composition. A micelle can have, for example, a surface that is composed of a charged outer layer. A micelle can encapsulate one or more biomolecules. For example, a micelle can encapsulate two or more proteins (such as a beta-casein protein and a kappa-casein protein). A micelle can have diameter of between about 10 nanometers (nm) and about 350 nm. A micelle can have a diameter of more than about 350 nm. A micelle can have a diameter of less than about 10 nm.

As used herein, the term “mutated” or “mutation” when applied to nucleic acid sequences generally refers to nucleotides in a nucleic acid sequence which may be inserted, deleted, or changed compared to a reference nucleic acid sequence. A single alteration may be made at a locus (a point mutation) or multiple nucleotides may be inserted, deleted, or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique 1:11-15, 1989, and Caldwell and Joyce, PCR Methods Applic. 2:28-33, 1992, each of which is incorporated herein by reference in their entireties for all purposes); and “oligonucleotide-directed mutagenesis” (a process which enables the generation of site-specific mutations in any cloned DNA segment of interest; see, e.g., Reidhaar-Olson and Sauer, Science 241:53-57, 1988, which is incorporated herein by reference in its entirety for all purposes).

As used herein, the term “percent sequence identity” or “identical” in the context of nucleic acid sequences generally refers to the residues in the two sequences which may be the same when aligned for maximum correspondence. The length of sequence identity comparison may be over a stretch of at least about nine nucleotides, e.g., at least about 20 nucleotides, at least about 24 nucleotides, at least about 28 nucleotides, at least about 32 nucleotides, or at least about 36 or more nucleotides. The length of a sequence identity comparison may be over a stretch of less than nine nucleotides. An algorithm may be applied to measure nucleotide sequence identity. For example, polynucleotide sequences can be compared using FASTA, Gap, or Bestfit, which are programs in Wisconsin Package Version 10.0, Genetics Computer Group (GCG), Madison, Wis. FASTA may provide alignments and percent sequence identity of the regions of the best overlap between the query and search sequences. See, e.g., Pearson, Methods Enzymol. 183:63-98, 1990 (which is incorporated here by reference). For example, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410, 1990; Gish and States, Nature Genet. 3:266-272, 1993; Madden et al., Meth. Enzymol. 266:131-141, 1996; Altschul et al., Nucleic Acids Res. 25:3389-3402, 1997; Zhang and Madden, Genome Res. 7:649-656, 1997, especially blastp or tblastn (Altschul et al., Nucleic Acids Res. 25:3389-3402, 1997 (each of which is incorporated herein by reference in their entireties for all purposes).

As used herein, the term “operably linked” expression control sequences generally refers to a linkage in which the expression control sequence is contiguous with the gene of interest to control the gene of interest, as well as expression control sequences that act in trans or at a distance to control the gene of interest. As used herein, the term “polynucleotide” or “nucleic acid molecule” generally refers to a polymeric form of nucleotides of around at least 10 bases in length. The term may include DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, or double-stranded, or circular.

As used herein, the term “recombinant” when referring to a nucleic acid (e.g., a gene) generally refers to a nucleic acid that has been removed from its naturally occurring environment, a nucleic acid that is not associated with all or a portion of a nucleic acid abutting or proximal to the nucleic acid when it is found in nature, a nucleic acid that is operatively linked to a nucleic acid which it is not linked to in nature, or a nucleic acid that does not occur in nature. The term “recombinant” can be used, e.g., to describe cloned DNA isolates, or a nucleic acid including a chemically-synthesized nucleotide analog. When “recombinant” is used to describe a protein, it can refer to, e.g., a protein that is produced in a cell of a different species or type, as compared to the species or type of cell that produces the protein in nature. As used herein, an endogenous nucleic acid sequence in the genome of an organism (or the encoded protein product of that sequence) may be deemed “recombinant” if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). For example, a promoter sequence can be substituted (e.g., by homologous recombination) for the native promoter of a gene in the genome of a host cell, such that this gene has an altered expression pattern. This gene may now become “recombinant” because it is separated from at least some of the sequences that naturally flank it. A nucleic acid may also be considered “recombinant” if it contains any modifications that do not naturally occur to the corresponding nucleic acid in a genome. For example, an endogenous coding sequence may be considered “recombinant” if it contains an insertion, deletion, or a point mutation introduced artificially, e.g., by human intervention. A “recombinant nucleic acid” may also include a nucleic acid integrated into a host cell chromosome at a heterologous site and a nucleic acid construct present as an episome.

As used herein, the term “recombinant host cell” (“expression host cell,” “expression host system,” “expression system” or simply “host cell”), generally refers to a cell into which a recombinant vector has been introduced. Such terms may refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell that resides in a living tissue or organism. Mammalian cells described herein can be genetically engineered to have a genome that is different from the genome of the naturally occurring cell. Such engineered cells can be termed recombinant mammary cells, expression mammary cells, etc. in accordance with the context hereof.

As used herein, the term “substantial homology” or “substantial similarity,” when referring to a nucleic acid or fragment thereof, generally refers to a nucleic acid or fragment thereof when optimally aligned with appropriate nucleotide insertions or deletions with another nucleic acid (or its complementary strand), there may be nucleotide sequence identity in at least about 75%, at least about 80%, at least about 85%, at least about 90%, at least about 95%, at least about 96%, at least about 97%, at least about 98%, or at least about 99% of the nucleotide bases, as measured by any well-known algorithm of sequence identity, such as FASTA, BLAST or Gap, as discussed above.

As used herein, the term “stringent hybridization” generally refers to a hybridization process which may be performed at about 25° C. below the thermal melting point (Tm) for the specific DNA hybrid under a particular set of conditions. “Stringent washing” may be performed at temperatures about 5° C. lower than the Tm for the specific DNA hybrid under a particular set of conditions. The Tm may be the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y., page 9.51, 1989, hereby incorporated by reference. For purposes herein, “stringent conditions” may generally refer to for solution phase hybridization as aqueous hybridization (e.g., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. Hybridization at 65° C. may occur at different rates depending on a number of factors including the length and percent identity of the sequences which are hybridizing. Alternatively, substantial homology or similarity may exist when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments may depend upon a number of different physical parameters. Nucleic acid hybridization may be affected by such conditions as salt concentration, temperature, solvents, the base composition of the hybridizing species, length of the complementary regions, or the number of nucleotide base mismatches between the hybridizing nucleic acids as well as other factors.

As used herein, the terms “synthetic” or “artificial” may be used interchangeably and generally refer to a molecule, compound, or composition of matter that is man-made and does not generally occur in such form or structure in nature. Modifications to the chemical structure, or physical structure of a naturally occurring molecule, compound or composition of matter, however small, may be included in the terms “synthetic” or “artificial” so long as such modifications do not generally occur in nature.

The term “oligonucleotide”, as used herein, generally refers to a nucleic acid molecule comprising at least one nucleotide that may have various lengths such as either deoxyribonucleotides or ribonucleotides or analogs thereof. An oligonucleotide may comprise at least about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 250, 300, 400, 500, 600, 700, 800, 900, 1,000, 5,000, 10,000, 50,000, 100,000 or more nucleotides. An oligonucleotide may comprise at most about 100,000, 50,000, 10,000, 5,000, 1,000, 900, 800, 700, 600, 500, 400, 300, 250, 200, 175, 150, 125, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or less nucleotides. An oligonucleotide may be unbound (e.g., in solution) or bound (e.g., chemically bonded to a substrate). Oligonucleotides may include one or more nonstandard nucleotide(s), nucleotide analog(s), modified nucleotides, or any combination thereof.

As used herein, the term “component of milk” or “milk component” or “milk nutrient” generally refers to a molecule, compound, element, or an ion present in a mammal-produced milk. Non-limiting examples of a component of milk, or a milk component, may include a fat or a lipid; a protein such as casein, or a whey protein; a mineral or ash, such calcium, phosphate, magnesium, sodium, copper, iron, zinc, manganese, and potassium; an amino acid such as tryptophan, threonine, leucine, isoleucine, methionine, lysine, cystine, phenylalanine, tyrosine, valine, histidine, arginine, alanine, aspartic acid, betaine, glycine, glutamic acid, proline, and serine.

As used herein, the term “essentially free of” or “substantially free of” a particular molecule, protein, oligosaccharide, lipid, cell, microbe, etc, generally may indicate that the composition is substantially devoid of such molecule, protein, oligosaccharide, lipid, cell, microbe, etc. Expressed in terms of purity, essentially free of, or substantially free of, generally refers to a situation in which the amount of such molecule, protein, oligosaccharide, lipid, cell, microbe, etc, may not exceed, by weight percentage, (about) 10%, (about) 5%, (about) 1%, or (about) 0.5%.

As used herein, the term “mammary cell(s)” generally refers to mammary epithelial cell(s), mammary-epithelial luminal cell(s), or mammalian epithelial alveolar cell(s), or any combination thereof. As used herein, the term “mammary-like cell(s)” generally refers to cell(s) having a phenotype/genotype similar (or substantially similar) to natural mammary cell(s) but is/are derived from non-mammary cell source(s). Such mammary-like cell(s) may be engineered to remove at least one undesired genetic component and/or to include at least one predetermined genetic construct that is typical of a mammary cell. Non-limiting examples of mammary-like cell(s) may include mammary epithelial-like cell(s), mammary epithelial luminal-like cell(s), non-mammary cell(s) that exhibits one or more characteristics of a cell of a mammary cell lineage, or any combination thereof. Further non-limiting examples of mammary-like cell(s) may include cell(s) having a phenotype similar (or substantially similar) to natural mammary cell(s), or more particularly a phenotype similar (or substantially similar) to natural mammary epithelial cell(s). A cell with a phenotype or that exhibits at least one characteristic similar to (or substantially similar to) a natural mammary cell or a mammary epithelial cell may comprise a cell (e.g., derived from a mammary cell lineage or a non-mammary cell lineage) that exhibits either naturally, or has been engineered to, be capable of expressing at least one milk component.

As used herein, the term “non-mammary cell(s)” may generally include any cell of non-mammary lineage. In an embodiment, a non-mammary cell can be any mammalian cell capable of being engineered to express at least one milk component. Non-limiting examples of such non-mammary cell(s) include hepatocyte(s), blood cell(s), kidney cell(s), cord blood cell(s), epithelial cell(s), epidermal cell(s), myocyte(s), fibroblast(s), mesenchymal cell(s), or any combination thereof. In some instances, molecular biology and genome editing techniques can be engineered to eliminate, silence, or attenuate myriad genes simultaneously.

As used herein, the term “attenuate” may generally refer to a functional deletion made to a gene sequence (or a sequence controlling the transcription of a gene sequence), which reduces or inhibits production of the gene product, or renders the gene product non-functional. In some instances, a functional deletion may include knockout mutation(s). Non-limiting examples of attenuation may include amino acid sequence changes by altering the nucleic acid sequence, placing the gene under the control of a less active promoter, down-regulation, expressing interfering RNA, ribozymes or antisense sequences that target the gene of interest, or through any other technique known in the art. In a non-limiting example, the sensitivity of a particular enzyme to feedback inhibition or inhibition caused by a composition that is not a product or a reactant (non-pathway specific feedback) may be lessened such that the enzyme activity is not impacted by the presence of a compound. In other instances, an enzyme that has been altered to be less active can be referred to as attenuated.

As used herein in the context of genetic engineering, the term “deletion” generally refers to removal of nucleotide(s) from nucleic acid molecule(s) or removal of amino acid(s) from protein(s), for example, the regions on either side being joined together. For instance, such methods can be used to edit the genetic loci of undesirable genes, to insert desirable genes into the mammalian cell, to make non-mammary cells into mammary-like cells, or any combination thereof. See, for example, Smith et al., entitled “Enabling large-scale genome editing at repetitive elements by reducing DNA nicking,” Nucleic Acids Research, Volume 48, Issue 9, 21 May 2020, Pages 5183-5195, https://doi.org/10.1093/nar/gkaa239, which is incorporated herein by reference in its entirety for all purposes.

Milk is a dairy product; a nutrient-rich liquid food produced by mammalian mammary glands during or after pregnancy and is a primary source of nutrition for infant mammals including humans. It is considered a balanced diet due to its unique beneficial properties attributed to its composition. Mammal- or mammalian-produced milk is a very complex fluid that includes several thousand components such as of lipids (mono-, di- and triglycerides, phospholipids, free fatty acids, cholesterol and cholesteryl esters), proteins (casein and whey proteins), vitamins (vitamin A, B1, B2, B5, B6, B12, C, D, E and K), minerals (calcium, potassium, magnesium, phosphate, sodium, chloride, citrates and calcium-phosphate), carbohydrates and sugars (glucose, lactose, galactose, and other oligosaccharides). However, the exact composition of milk varies from species to species. Other components of milk consist of stem cells, mammary gland cells, white blood cells, enzymes, bacteria, and immunoglobulins. Besides being rich in nutrients, milk is a source of dairy products such as yogurt, cream, cheese, butter, and ice cream.

Global per capita consumption of milk is estimated at 108 liters per year, with milk consumption alone estimated at 81 billion liters per year. The global dairy market volume is estimated to be 216 metric tons (of which 54% market share is taken up by liquid milk) with a global annual sale of 674 billion USD projected to increase. A 22% increase in milk production from 2017 is projected by 2027. World consumption of fresh dairy products and processed dairy products is poised to grow by 2.1% and 1.7% per year respectively over the next decade. The largest share of milk and dairy product consumption is in the form of fresh dairy products, taking up about 50% of the world's total milk production and is expected to increase to 52% in the next ten years due to rising milk consumption in developing countries. Per capita consumption of dairy products in developing countries is expected to increase by an average of 0.5% per year for whole milk powder, 1.1% for skim milk powder, 0.8% for cheese, 1.7% for butter, and 1.9% for fresh dairy products. In developed countries, increasing per capita consumption of dairy products is also anticipated with some dairy product use further increasing in the manufacturing sector such as skim milk powder in confectionary, infant formula, research, and bakery products.

Although mammal-produced milk, such as bovine milk, is considered by many to be an ideal source of nutrition, various milk alternatives to mammal- or mammalian-produced milk bovine alternative sources of milks have been pursued for reasons related to mammal- or mammalian-produced milk's allergenicity, and lactose intolerance of certain components. These suboptimal attributes may not be easily correctable as milk is a complex mixture of several thousand components and cannot easily be altered. Alternative sources of milk may comprise plant-based milk such as soy, oats, almonds, cashew nuts, coconut, flaxseed, rice and tiger nut milk or milk produced by recombinant or chemical synthesis methods. U.S. sales of lactose-free dairy products in 2015 were $6.7 billion, and U.S. sales of plant- or nut-based dairy-like products in 2015 were $13.7 billion. Dairy alternatives fall short both in flavor and in functionality; moreover, a large part of the industrial and cultural significance of dairy milk stems from its usefulness in derivative products, such as cheese, yogurt, cream, or butter. Non-dairy plant-based milks, while addressing environmental and health often fail to form such derivative products when subjected to the same processes used for dairy milk.

Dairy alternatives often do not provide a sufficient milk replacement for newborn or infant mammals whose sole dietary component is milk. Consequently, infant mammals that cannot get enough breast milk either because their mothers cannot express enough milk or are not able to breastfeed the infants are fed with infant formula as an alternative to breast milk. The composition of infant formula cannot match the complexity of breast milk which may be particularly relevant for rare or endangered mammals. In one example human breast milk is a rich source of human milk oligosaccharides (HMOs) that are critical for early development and health of human infants. HMOs act as prebiotics, have antibacterial, and immunomodulating properties. These health benefits are the synergistic effects of over 150 different HMOs identified in human milk. Compared to natural human breast milk, two HMO's (lacto-N-neotetraose and 2′-fucosyllactose) are available commercially for addition to human infant formula. Human infants fed with human infant formula are at higher risk of developing conditions like diabetes, obesity, respiratory and immune disorders later in life.

There is also widespread concern over the impact of conventional milk production on animal welfare and the environment. For example, the environmental impact resulting from dairy effluent can result in significant levels of nitrate which has the potential to contaminate groundwater. Groundwater forms the main source of water supply for many towns and farms where surface water supplies are limited. In the US, half the population relies completely or partially on groundwater. Further, dairy cattle such as cows, goats, camels and others require intensive resources. Livestock consume 70% of all wheat, corn, and other grain produced in the United States alone. Livestock is responsible for 18% of Green House Gas (GHG) emissions, use 30% of Earth's terrain, 70% of arable land, and 8% of freshwater globally. Dairy cattle are predominantly grazing unculates or hooved animals that can cause damage to soils and grasslands. In 2017, 278 million dairy cows were predicted to exist in the world that produced 828 million tons (72.5 billion liters) of milk. With the increasing population of the world, milk consumption and demand is predicted to rise. Meeting this demand using traditional livestock agriculture and increasing dairy cattle may result in greater ecological and environmental concerns.

While further industrializing the dairy industry may consolidate some of the resource utilization, factory farming and poor animal welfare conditions in livestock agriculture are a cause for foodborne illnesses and dairy contaminants. Contaminants encountered in milk and milk products may be derived from animal husbandry practices and may include pathogens, pesticide residues, heavy metals, and aflatoxin M1. The presence of foodborne pathogens in milk may be due to direct contact with contaminated sources in a dairy farm environment or from excretion from the udder of an infected animal. Outbreaks of disease in humans have been traced to the consumption of both unpasteurized and pasteurized milk. A more efficient, safer, and healthier method of dairy production than current methods of production may be needed.

Currently, the methods employed for milk production outside of traditional agriculture methods are based on producing the components (casein, whey proteins, etc.) by recombinant based methods using bacterial, yeast or mammalian expression systems or using plants as alternate source of milk. Milk produced by these methods lack most of the components of milk (protein and fatty content, oligosaccharides, etc.). While methods for isolating and differentiating mature breast luminal and epithelial cells from mesenchymal stem cells from breast tissue or milk are known, it is difficult to produce large volumes of milk using such methods and the profile of the milk produced varies too much from naturally produced milk. Therefore, there exists a need for the development milk and its derivative dairy products which minimizes foodborne pathogens, has a lower environmental impact, which retains the ability to be used for derivative or downstream applications of dairy milk, and which provide a similar nutritional profile as a mammal- or mammalian-produced milk. Provided herein are systems and methods for producing cultured dairy for food consumption.

### Nutrient Compositions

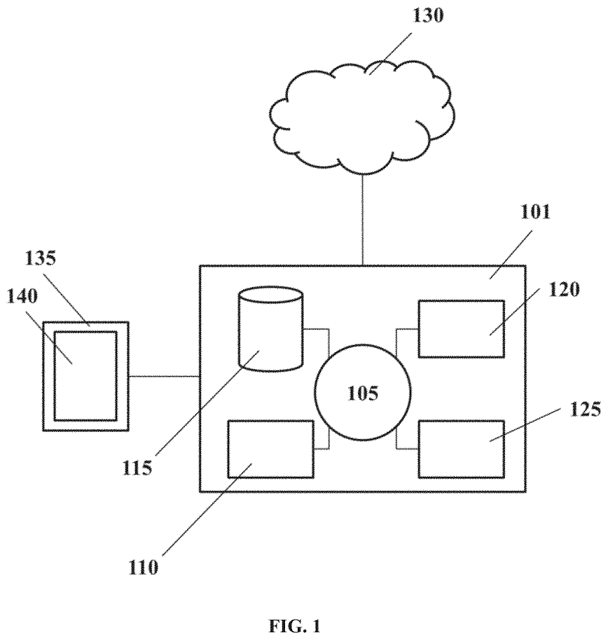

Aspects of the present disclosure may comprise nutrient compositions (such as liquid nutrient compositions or liquid compositions). In some embodiments, the nutrient compositions (such as liquid nutrient compositions or liquid compositions) may be obtained from an in vitro culture of mammary cells or mammary-like cells (for example, as shown in the non-limiting example schematic of FIG. 1). In some embodiments, the nutrient composition(s) (such as liquid nutrient composition(s) or liquid composition(s)) or milk product(s) may be substantially free of immunoglobulin(s) (or antibody or antibodies), substantially free of whole cell(s), or substantially free of bacterial microbe(s), or a combination thereof. The mammary cells or mammary-like cells can be obtained by differentiating mammalian stem cell composition(s) (e.g., mammalian mesenchymal stem cell composition(s) such as described herein) (for example, as shown in the non-limiting example schematic of FIG. 1). In some embodiments, the stem cell(s), mammary cell(s), or mammary-like cell(s) can be genetically engineered to enhance or reduce any component produced by the cells resulting in a composition having any predetermined composition comprising at least one protein, at least one oligosaccharide, or at least one fat or lipid, or any combination thereof (such as described herein). The nutrient composition(s) produced by the in vitro culture of such mammary cells or mammary-like cells can be supplemented with ingredient(s) to alter its flavor, texture, or nutritional benefits. In some embodiments, mammalian mesenchymal stem cells may be isolated and cultured, e.g., for 48 hours or 96 hours. The mammalian mesenchymal stem cells may proliferate in a growth medium, e.g., for up to 7 days. In some embodiments, these cells may be grown, e.g., in a 2D or 3D culture. In some embodiments, these cells may be subsequently differentiated into mammary epithelial and luminal cells and seeded in a 2D or 3D culture. Cells seeded on scaffolding in 2D or 3D culture may be cultured in a bioreactor vessel and further differentiated into lactating cells using a lactation media (for example, as shown in the non-limiting example schematic of FIG. 2). Components of lactation may be filtered out to create a liquid composition or milk nutrient composition (such as described herein).

### Food Composition

Aspects of the present disclosure may provide compositions, kits, systems, and methods for producing food compositions (such as food products (e.g., in vitro engineered)). In some embodiments, a food composition or food product may be any composition capable of being ingested or metabolized by human(s) or other animal(s) to give energy and build tissue. A food composition or food product may be eaten or drunk by human(s) or other animal(s) for nutrition or pleasure. A food composition or food product may comprise carbohydrates, fats, proteins, or water, or any combination thereof. A food composition or food product may be combined with or added to other ingredients to generate compositions that can be ingested by human(s) or other animal(s). A food composition or food product may comprise a dairy product (or dairy composition). A dairy product (or dairy composition) may refer to milk (e.g., whole milk, partly skimmed milk, skim milk, cooking milk, condensed milk, flavored milk, goat milk, sheep milk, dried milk, evaporated milk, milk foam), or product(s)/composition(s) derived from milk, including but not limited to yogurt (e.g., whole milk yogurt, low-fat yogurt, nonfat yogurt, Greek yogurt (e.g., strained yogurt with whey removed), whipped yogurt, goat milk yogurt, Labneh, sheep milk yogurt, yogurt drinks (e.g., whole milk Kefir, low-fat milk Kefir, Lassi), cheese (e.g., whey cheese such as ricotta and mozzarella, semi-soft cheese such as Havarti and Munster, medium-hard cheese such as Swiss and Jarlsberg, hard cheese such as Cheddar, soft ripened cheese such as Brie and Camembert, cottage cheese, cream cheese, curd), cream (e.g., whipping cream, coffee whitener, coffee creamer, sour cream, creme fraiche), frozen confections (e.g., ice cream, smoothie, milk shake, frozen yogurt, sundae), butter, infant formula, weight loss beverages, nutritional beverages, pudding, buttermilk, milk protein concentrate, whey protein concentrate, whey protein isolate, casein concentrate, casein isolate, skim milk powder, whole milk powder, nutritional supplements, texturizing blends, flavoring blends, or coloring blends. A dairy product (or dairy composition) may comprise an animal component grown in vitro such as a cultured cell or cultured cell component. A dairy product (or dairy composition) may be used to generate any kind of food composition or food product originating from or similar to the milk of an animal or its derivates. A dairy product (or dairy composition) may comprise a hybrid food composition or food product comprising a plant-originated substance and a cultured dairy, cells, or substances interconnected with the plant-originated substance to form a unified food composition or food product with an improved organoleptic and nutritional value compared with a sole plant-originated substance. A dairy product (or dairy composition) may be free of bodily fluids e.g., saliva, serum, plasma, mucus, urine, feces, tears, milk etc. or may comprise a bodily fluid.

In some embodiments, a cultured dairy product may comprise an in vitro cell culture of animal cells such as mammalian epithelial or luminal cells or their secretions (such as described herein). A cultured cell may refer to a cell grown under controlled conditions, outside their natural environment in in vitro conditions. A cultured dairy product may comprise a mammal-produced milk which generally refers to a milk produced by a mammal. A dairy product may comprise a processed mammal-produced milk which may refer to a mammal-produced milk that is processed using one or more steps known in the dairy industry such as homogenization, pasteurization, irradiation, or supplementation. A cultured dairy product may comprise a component of milk or milk component which may refer to a molecule, compound, element, or an ion present in a mammal-produced milk. A component of milk may be mammal-derived components which may comprise a molecule, element, ion, or compound such as a protein, a lipid, a mineral or ash, or a nucleic acid obtained from the body of a mammal or a molecule obtained from a fluid or solid produced by a mammal. Non-limiting examples of a component of milk (or a milk component) may comprise a fat or a lipid, a protein (such as casein or whey protein), a mineral or ash (such calcium, phosphate, magnesium, sodium, copper, iron, zinc, manganese, and potassium), an amino acid (such as tryptophan, threonine, leucine, isoleucine, methionine, lysine, cystine, phenylalanine, tyrosine, valine, histidine, arginine, alanine, aspartic acid, betaine, glycine, glutamic acid, proline, and serine), or any combination thereof. A component of milk may comprise a concentration of a component in a mammal-produced milk which may refer to the concentration of a component in the milk produced by a mammal or the mean concentration of a component in milk produced by a population of mammals of the same species. A component of milk may comprise a milk protein which generally refers to a protein that can be found in a mammal-produced milk or a protein having a sequence that that may be substantially similar to the sequence of a protein that can be found in a mammal-produced milk. In some cases, a milk protein may comprise β-casein, κ-casein, α-S1-casein, α-S2-casein, α-lactalbumin, β-lactoglobulin, lactoferrin, transferrin, serum albumin, or any combination thereof. A milk protein may comprise a milk protein component such as the proteins or protein equivalents and variants found in milk such as casein, whey or a combination of casein and whey, including their subunits, which may be derived from various sources. A casein protein generally refers to a family of proteins present in mammal-produced milk and capable of self-assembling with other proteins in the family to form micelles and/or precipitate out of an aqueous solution at an acidic pH. Non-limiting examples of casein proteins may include β-casein, κ-casein, α-S1-casein, and α-S2-casein which are found in bovine milk. A protein, or a plurality of proteins, may be encapsulated with a micelle, such as an approximately spherical supramolecular structure that exists as a dispersion within a composition. For example, a micelle can have a surface that is composed of a charged outer layer. In an example, a micelle may encapsulate a protein such as a β-casein protein or a κ-casein protein among others.

In some embodiments, a dairy product or dairy composition may comprise nutritional additive(s) comprising vitamin(s) or mineral(s) or both. The dairy product or dairy composition may optionally further comprise a flavor-enhancing additive (such as a sweetening agent). A sweetening agent may comprise a saccharide (such as a monosaccharide, a disaccharide, or a polysaccharide) or an artificial sweetener (such as a small molecule artificial sweetener or a protein artificial sweetener) that, when added to a composition, makes the composition taste sweet when ingested by a mammal, such as a human. Cultured cells or tissues may be combined with at least one other ingredient, for example, to obtain a food composition or food product having a desired texture, moisture retention, product adhesion, or any combination thereof. Ingredients that provide flavor, texture, or other culinary properties may be added to a dairy product or dairy composition. An ingredient may comprise a binder, filler, or extender. A filler or binder may comprise a non-dairy substance comprising carbohydrate(s) such as a starch. Non-limiting examples of filler(s) or binder(s) may include potato starch, flour, eggs, gelatin, carrageenan, or tapioca flour, or a combination thereof. An extender may have a high protein content. Non-limiting examples of extender(s) may comprise soy protein, milk protein, or dairy-derived protein, or a combination thereof. Ingredients that provide flavor, texture, or other culinary properties may be added to a dairy product or dairy composition. For example, extracellular matrix proteins may be used to modulate structural consistency and texture contributing to the taste of the desired food composition(s) or food product(s). In some instances, nutrients such as vitamins that are normally lacking in dairy products from whole animals may be added to increase the nutritional value of the dairy product. This may be achieved either through straight addition of the nutrients to a growth medium or by alternative methods. For example, the enzymes responsible for the biosynthesis of a particular vitamin, such as Vitamin D, A, or the different Vitamin B complexes, may be transfected into the cultured mammary cells to produce the particular vitamin within those cells.

In some embodiments, agents, compounds, molecules, or ingredients can be added to the liquid compositions described herein. For example, and without limitation, the following can be further added to, or supplementing, a liquid composition or milk product described herein: at least one of a mineral, at least one of a vitamin, at least one of an immunoglobulin, at least one of an amino acid, at least one of a carbohydrate, at least one of an oligosaccharide, at least one cell, at least one microbe, or any combination thereof. At least one vitamin, may include but is not limited to, at least one of vitamin A, vitamin B, vitamin C, vitamin D, vitamin K, vitamin E, thiamine, niacin, biotin, riboflavin, folates, and pantothenic acid. Vitamin B may include, but is not limited to, at least one of thiamin, riboflavin, niacin, biotin, pantothenic acid, folate, pyridoxine (and related substances, such as vitamin B₆) and cobalamin (and its derivative, vitamin B₁₂). Vitamin A can be present in a variety of forms and may include at least one of retinol, retinal, retinoic acid, retinyl esters as well as provitamin A carotenoids such as β-carotene. Vitamin E can include at least one of two groups of compounds: tocopherols (α-, β-, γ- and δ-) and tocotrienols (α-, β-, γ- and δ-). Vitamin K can include at least one of phylloquinone (vitamin K₁ and menaquinone (vitamin K₂). Vitamin D can include at least one of cholecalciferol (vitamin D₃), and ergocalciferol (vitamin D₂). At least one mineral may comprise, but is not limited to, at least one of calcium, phosphate, magnesium, sodium, copper, iron, zinc, manganese, and potassium. At least one amino acid may include, but is not limited to, at least one of tryptophan, threonine, leucine, isoleucine, methionine, lysine, cystine, phenylalanine, tyrosine, valine, histidine, arginine, alanine, aspartic acid, betaine, glycine, glutamic acid, proline, serine, taurine, carnitine, arginine, and inositol. At least one immunoglobulin may include, but is not limited to, immunoglobulin A (IgA), Immunoglobulin G (IgG), and immunoglobulin M (IgM). The at least one bacterial microbe that can be further included in the compositions may include but are not limited to, at least one of Pseudomonas, Staphylococcus, Streptococcus, Elizabethkingia, Variovorax, Bifidobacterium, Flavobacterium, Lactobacillus, Stenotrophomonas, Brevundimonas, Chryseobacterium, and Enterobacter. See, for example, Murphy et al. (2017), entitled “The composition of human milk and infant fecal microbiota over the first three months of life: a pilot study” Scientific Reports, 7(1), pp. 1-10, which is incorporated herein by reference in its entirety for all purposes. The above list of ingredients for supplementing the liquid compositions or milk product described herein is not exhaustive and other ingredients can be added to thereto as desired.

In some embodiments, at least one Human Milk Oligosaccharide (HMO), or glycan, can be added to the liquid compositions described herein. There exist approximately 200 HMOs and HMOs may be structurally complex sugars unique to human breast milk. They can serve as prebiotics, which are substrates for fermentation processes by intestinal microbes, inducing the growth or activity of beneficial bacteria. HMOs can act as antiadhesive agents that inhibit pathogen adhesion to mucosal surfaces, preventing colonization and as antimicrobials by preventing proliferation of certain bacteria. HMOs may be composed out of five different monosaccharides, the structural complexity of HMOs encountered in human milk is unique to humans. The monosaccharides that are used as building blocks for HMOs may be glucose (Glc), galactose (Gal), N-Acetyl-Glucosamine (GlcNAc), fucose (Fuc), and sialic acid (Neu5Ac). These single monosaccharides may be conjugated via several linkage types (e.g., glycosidic bonds). With exceptions, HMOs structures may follow a strict building plan. Each of the HMOs structure may start with a lactose unit “Gal (β1-4) Glc” which results from formation of a β1-4 glycosidic linkage between galactose and glucose catalyzed by the lactose synthase protein complex. Several tri-saccharides can be synthesized by appending either galactose or fucose to the reducing or non-reducing end of the lactose residue, which is performed through galactosyl- or fucosyl-transferase activity. Resulting components may be for example, lacto-N-neoetraose (LNnT), 3′-galactosyllactose (Gal(β1-3)Gal(β1-4)Glc), 4′-galactosyllactose (Gal(β1-4)Gal(β1-4)Glc), 6′-galactosyllactose (Gal(β1-6)Gal(β1-4)Glc), 2′-FL (Fuc(α 1-2)Gal(β1-4)Glc), and 3-fucosyllactose (3′-FL) (Gal(β1-4)[Fucα1-3]Glc). If sialic acids are connected to the non-reducing end of lactose via sialyl-transferases, 3′-sialyllactose (3′-SL; Neu5Ac (α 2-3) Gal(131-4) Glc) and 6′-sialyllactose (6′-SL) (Neu5Ac (α 2-6) Gal((31-4) Glc) are formed. Further elongation of lactose via the free 3-OH group of galactose can occur by addition of Gal (β1-x) GlcNAc units of either type I (Gal (β1-3) GlcNAc, Lacto-N-biose) or type II (Gal (β1-4) GlcNAc, N-Acetyllactosamine). These core structures may be linear or branched and can be further decorated with fucoses or sialic acid residues. The cellular localization of HMOs synthesis in the mammary gland epithelium is believed to be the Golgi apparatus. At least one of the HMOs can be added to the liquid compositions described herein. Combinations of two or more HMOs are also contemplated to be included in the liquid composition or milk product described herein. HMOs of high purity are readily available from various commercial suppliers. For example, DuPont Nutrition & Bioscience, BASF Corporation, and Lonza Specialty Ingredients each supply various purified and food quality HMOs.

In some embodiments, bioactive lipids such as prostaglandins (PGs, including PGE2, PGD2, PGF2, PGI2), and thromboxane A2 are, which naturally are synthesized from arachidonic acid by cyclooxygenases, can be added to the liquid composition described herein. Such bioactive lipids can be made through methods that are known in the art or can be purchased from commercial sources. The following can be added to or supplemented in the liquid compositions described herein but are not limited: free amino acids, creatine, carnitine, Nucelotides (cytidine-(CMP), uridine-(UMP), adenosine-(AMP), guanosine-(GMP), inosine-monophosphate (IMP), and cytidine 5′-diphosphate (CDP), Polyamines such as putrescine, spermidine, spermine.

In some embodiments, other ingredients can be added to the liquid composition or milk product described herein to enhance, alter, or remove at least one characteristic of the liquid composition. For example, and without limitation, such other supplemental agents, compounds, molecules, or ingredients and the like may include flavoring agents, coloring agents, aroma agents, sweetening agents, and texturing agents vanilla, strawberry, chocolate, or durian. There exist myriad such agents, each intended for different purposes and possessing different chemical and biochemical properties, readily available to food scientists. Non-limiting examples of flavoring agents that can be added to the liquid composition or milk may include at least one natural flavoring agent or at least one synthetic flavoring agent, such as vanilla extract, vanilla essence, chocolate, sesame, durian, corn, yam, mushroom, coffee, banana, malt, cinnamon, turmeric, caramel, avocado, kiwi, almond, or mocha. Non-limiting examples of coloring agents that can be added to the liquid composition or milk may include at least one natural coloring agent or at least one synthetic coloring agent, such as E102, E104, E110, E122, E127, E129 E124, E131, E132, E133, E142, or E143. Non-limiting examples of aroma agents that can be added to the liquid composition or milk may include at least one natural aroma agent or at least one synthetic aroma agent, such as 2,5-dimethyl-4-hydroxy-3(2H) furanone (furaneol), butanedione (diacetyl) CH₃COCOCH₃, methylpyrazine, trimethyloxazole H3C, 2-acetylthiophene, or 2-actylthiazole. Non-limiting examples of sweetening agents that can be added to the liquid composition or milk may include at least one natural sweetening agent or at least one synthetic sweetening agent, such as stevia, erythritol, xylitol, yacon syrup, acesulfame potassium, aspartame, cyclamate, mogrosides, saccharin, sucralose, sugar alcohols, thaumatin (E957), isomalt (E953), or maltitol (E965). Non-limiting examples of texturing agents that can be added to the liquid composition or milk may include at least one natural texturing agent or at least one synthetic texturing agent, such as lecithin, cellulosic derivatives, pectin, gelatin, xanthan gum, starch, inulin, silicones, carrageenan, and phosphates.

Any of the above ingredients that is a milk component can be obtained from natural sources, and then isolated and purified from the natural environment; or can be made synthetically, e.g., from chemical synthesis or from using recombinant DNA methodology. For use in supplementing the liquid compositions described herein, the milk components that are synthesized may have substantial homology or be substantially identical to the respective naturally occurring component. The synthesized milk components that are supplemented into the liquid compositions described herein may be at least about 50%, 55%, 60%, 65%, 70%, 75%, 80%, 85%, 90%, 95%, 96%, 97%, 98%, 99%, 99.9%, or 100% homologous to their respective naturally occurring component. The synthesized milk components that are supplemented into the liquid compositions described herein may be less than about 50% homologous or identical to their respective naturally occurring component.

A liquid composition or milk obtained using aspects of the present disclosure can be customized to produce a composition having a desired level of at least one fat or lipid, a desired level of at least one protein, a desired level of at least one oligosaccharide, a desired level of at least one mineral, or the like. For example, and without limiting the disclosure, the liquid composition or milk can be customized to produce a high protein, low-fat, product; a product substantially free of lactose; a product substantially free of a whey protein; a product with a high fat content; a product of high fat and low sugar, etc. The described liquid composition or milk product can be customized to obtain any combination of a higher, or a lower, level of any milk component.

In some embodiments, certain milk components of the compositions (e.g., liquid compositions) produced by the in vitro culture of mammary cells, or mammary-like cells can be isolated or purified. For example, such milk components can include casein or caseinate product(s), including micellar casein protein, sodium caseinate, calcium caseinate, casein, A2 milk, rennet casein, and acidic casein. Any or all of these components can be isolated or purified from the composition (e.g., liquid). The compositions (e.g. liquid compositions) produced by the in vitro culture of mammary cells, or mammary-like cells, described herein can be used to produce dry products such as a dry buttermilk product, a nonfat dry milk, a milk permeate powder, a milk protein concentrate (for example, with approximately 82.5% protein), a milk protein isolate (for example, with approximately 90% protein), a dry whole milk, a skim milk powder, or a whole milk powder. Other milk components of the liquid compositions produced by the in vitro culture of mammary cells, or mammary-like cells, described herein that can be isolated or purified, include whey protein and products: whey protein powder, whey protein concentrate, whey protein isolate, hydrolyzed whey protein, dry whey (e.g., for human consumption), sweet whey powder, acid whey powder, delactosed whey, demineralized whey powder ingredient, whey permeate (e.g., high lactose dairy product solids), and whey peptides.

In some embodiments, a cultured dairy product may be produced by culturing cells in vitro. A cell may comprise a cell membrane, at least one chromosome, composed of genetic material, cytoplasm, and various organelles which are adapted or specialized to perform one or more vital functions, such as energy and proteins synthesis, respiration, digestion, storage and transportation of nutrients, locomotion, or cell division. A cell may comprise one or a plurality of cells. A cell may comprise a somatic cell, a terminally differentiated cell, a stem cell, or a germ cell. A somatic cell may be any cell forming the body of an organism that are not germline cells. Mutations in somatic cells may affect the individual organism but are not passed onto offspring. A terminally differentiated cell may refer to any cell that in the course of acquiring specialized functions, is not able to transform into other types of cells. These cells may constitute most of the mammalian body and may be unable to proliferate.

In some embodiments, the liquid composition produced by the in vitro culture of mammary cells, or mammary-like cells, can be readily substituted for natural mammalian milk into manufacturing processes for cheese, yoghurt, ice cream and many other milk-based products. The liquid composition produced by the in vitro culture of mammary cells, or mammary-like cells can be further modified to change the flavor profile of the liquid composition to enhance the quality of the desired derived product(s).

In some embodiments, a cell may be derived from any non-human animals such as mammals (e.g. cattle, buffalo, pigs, sheep, deer, etc.), birds (e.g. chicken, ducks, ostrich, turkey, pheasant, etc.), fish (e.g. swordfish, salmon, tuna, sea bass, trout, catfish, etc.), invertebrates (e.g. lobster, crab, shrimp, clams, oysters, mussels, sea urchin, etc.), reptiles (e.g. snake, alligator, turtle, etc.), or amphibians (e.g. frogs).

In some embodiments, a cultured dairy product may be produced by culturing cells in vitro. A cell may comprise a cell membrane, at least one chromosome, composed of genetic material, cytoplasm, and various organelles which are adapted or specialized to perform one or more vital functions, such as energy and proteins synthesis, respiration, digestion, storage and transportation of nutrients, locomotion, or cell division. A cell may comprise one or a plurality of cells. A cell may comprise a somatic cell, a terminally differentiated cell, a stem cell, a germ cell, or other cell type. A somatic cell may be any cell forming the body of an organism that are not germline cells. Mutations in somatic cells may affect the individual organism but are not passed onto offspring. A cell may comprise myoepithelial, cuboidal, leukocytes, epithelial cells, luminal cells, basal cells, alveolar cells, ductal cells, basal stem cells, luminal stem cells, epithelial cap cells, luminal epithelial cells, epithelial body cells, vascular endothelial cells, immune cells, fibroblasts, luminal progenitor cells, basal progenitor cells, or a mammary cell.

In some embodiments, a cell may comprise a cell with a mammary cell with a mammary cell lineage. A mammary cell lineage may comprise a precursor cell. A mammary cell may comprise a mammary epithelial cell or mammary-like cell. A cell may be a mammalian cell. A mammalian cell may comprise mammary epithelial cells obtained, or mammary cells differentiated from stem cells obtained, from any of the class Mammalia origin—for example, any member of the group of vertebrate animals in which the young are nourished with milk from special mammary glands of the mother. Members of the class Mammalia include but are not limited to, human; ungulate, including, but not limited to, bovine, including domestic dairy cow, bison, goat, sheep, buffalo, yak, horse, donkey, zebu, reindeer, deer, pig, peccarie, hippopotamus, camel, chevrotain, deer, giraffes, pronghorn, antelope, elk, moose, zebra, rhinoceros, and cattle, elephant; canine (family Canidae) including, but not limited to, domestic dog, coyote, fox, wolf, and dingo; feline (family Felidae) including, but not limited to, lion, tiger, snow leopard, jaguar, panther, mountain lion, domestic cat, bobcat, and lynx; aquatic mammals including, but not limited to, cetaceans such as whales, narwhals, dolphins, and porpoise; pinnipeds including, but not limited to, seal, walrus, and sea lions; marsupials including, but not limited to, kangaroo, wallaby, koala, phalangeriform, opossum, wombat, Tasmanian devil, dunnart, potoroo, and cuscus; rodent; bat; squirrel, panda, mouse, hedgehog, monkey, chimpanzee, ox, raccoon, horse, mole, armadillo, beaver, gibbon, gopher, Llama, orangutan, orca, otter, polar bear, porcupine, puma, rabbit, rat, and weasel. For bovine, the mammary cells or mammary-like cells can be obtained, or mammary cells differentiated from stem cells obtained, from the cows of the species Holstein-Friesian, Ayrshire, Brown Swiss, Guernsey, and Jersey. As illustrated in FIG. 1, mammary stem cells may be separated from milk, aliquoted, and growth media added to cause proliferation to obtain mammary or mammary-like cells. Media may be changed to differentiation media where the mammary stem cells may differentiate into mature mammary cells.

In some embodiments, a cell may comprise an adipocyte. An adipocyte may be a cell primarily composed of adipose tissue, specialized in synthesizing and storing energy as fat. Adipocytes may be derived from mesenchymal stem cells through adipogenesis. Adipocytes may be white adipocytes, which store energy as a single large lipid droplet and have important endocrine functions, and brown adipocytes which store energy in multiple small lipid droplets but specifically for use as fuel to generate body heat. A cell-derived dairy product may comprise one cell type, such as a mammary epithelial cell, or a heterogeneous co-culture composition, such as a mammary epithelial cell and an adipocyte composition. A plurality of single cell types may be cultured individually and then combined into a desired product. A dairy product may be derived from mammary epithelial cells grown ex vivo and may include fat cells derived also from any non-human animals. A ratio of mammary cells to fat cells may be regulated to produce a dairy product with optimal flavor and health effects. A dairy product may be derived from mammary cells, stem cells, mammary epithelial cells, myoepithelial cells, luminal cells, basal cells, alveolar cells, ductal cells or a combination thereof. A dairy product may be derived from another cell type. A dairy product may be derived from a mammary cell or mammary-like cell such as a mammary progenitor cell or a mammary stem cell.

In some embodiments, differentiation may refer to the process during which young, unspecialized cells take on individual characteristics and reach their specialized form and function. Cell differentiation may allow a single cell and genotype to result in tens to hundreds of different cell types and phenotypes. Through differentiation a totipotent cell may move through pluripotency or multipotency, eventually reaching a lineage committed state. A cell may comprise a stem cell which may be any unspecialized cell capable of renewing themselves through cell division which have the developmental potential to differentiate into multiple cell types. A stem cell may be any unspecialized cell capable of self-renewal through cell division which may have the developmental potential to differentiate into multiple cell types, without a specific implied meaning regarding developmental potential, for example a stem cell can be totipotent, pluripotent, multipotent, etc. A stem cell may be a cell capable of proliferation and giving rise to more such stem cells while maintaining its developmental potential. A stem cell may refer to any subset of cells that have the developmental potential, under particular circumstances, to differentiate to a more specialized or differentiated phenotype, and which retain the capacity, under certain circumstances, to proliferate without substantially differentiating. A stem cell may refer to a naturally occurring parent cell whose descendants (progeny cells) specialize, often in different directions, by differentiation, e.g., by acquiring completely individual characters, as occurs in progressive diversification of embryonic cells and tissues. Some differentiated cells may have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype.

In some embodiments, ectopic differentiation factors may induce differentiation in a transient and non-integrative manner using non-native induction through biochemical systems. Ectopic differentiation factors may comprise nucleic acids, polypeptides, small molecules, growth factors, or any combination thereof. A cultured stem cell or progenitor cell may be differentiated by arresting the cell cycle of the stem cell or progenitor cell. Ectopic differentiation factors may arrest the cell cycle of cells by reducing or removing growth factors. Ectopic differentiation factors may arrest the cell cycle of cells through reducing or removing growth factors from a subset of cultured cells. Growth factors may be reduced or removed from a subset of cultured cells. Self-renewal and pluripotency of stem cells may be governed by extrinsic signals mediated by an endogenous pluripotency gene regulatory network consisting of a set of core transcription factors such as Oct3/4 or Sox2. Transcription factor interactions may regulate genomic functions by establishing both negative and positive feedback loops and transcription by recruiting activators and repressors to modulate the transcriptional machinery. Maintaining stem cell characteristics of self-renewal and differentiation in pluripotent stem cells may require distinct extrinsic signaling pathways including leukemia inhibitory factor (LIF), FGF/extracellular signal-regulated kinase (ERK) pathway, Wnt/glycogen synthase kinase 3 (GSK3), and transforming growth factor-beta (TGF-β) signaling Growth factors which may influence the differentiation of stem or progenitor cells may comprise LIF, FGF, BMP, activin, MAPK, and TGF-β.

In some embodiments, a stem cell may comprise a non-mammary stem cell. For example, a non-mammary stem cell may comprise a non-mammary adult stem cell. A stem cell may be any unspecialized cell capable of self-renewal through cell division which may have the developmental potential to differentiate into multiple cell types, without a specific implied meaning regarding developmental potential. A stem cell may be totipotent, pluripotent, multipotent, oligopotent, or unipotent. A stem cell may be a cell capable of proliferation and giving rise to more such stem cells while maintaining its developmental potential. A stem cell may refer to any subset of cells that have the developmental potential, under particular circumstances, to differentiate to a more specialized or differentiated phenotype, and which retain the capacity, under certain circumstances, to proliferate without substantially differentiating. A stem cell may refer to a naturally occurring parent cell whose descendants (progeny cells) specialize, often in different directions, by differentiation, e.g., by acquiring completely individual characters, as occurs in progressive diversification of embryonic cells and tissues. Some differentiated cells may have the capacity to give rise to cells of greater developmental potential. Such capacity may be natural or may be induced artificially upon treatment with various factors.

In some embodiments, a stem cell may comprise an embryonic stem cell, animal stem cell, adult stem cell, induced pluripotent stem cell, reprogrammed stem cell, mesenchymal stem cell, hematopoietic stem cell, or a progenitor cell. An embryonic stem cell may refer to embryonic cells capable of differentiating into cells of all three embryonic germ layers (the endoderm, ectoderm and mesoderm), or remaining in an undifferentiated state. The embryonic stem cells may comprise cells which are obtained from the embryonic tissue formed after gestation (e.g., blastocyst) before implantation of the embryo, such as a pre-implantation blastocyst, extended blastocyst cells which are obtained from a post-implantation/pre-gastrulation stage blastocyst, embryonic germ cells which are obtained from the genital tissue of a fetus, and cells originating from an unfertilized ova which are stimulated by parthenogenesis (parthenotes). An embryonic stem cell has unlimited self-renewal ability and pluripotent differentiation ability. An adult stem cell may be any stem cell derived from a somatic tissue of either a postnatal or prenatal animal. An adult stem cell may be capable of indefinite self-renewal while maintaining its undifferentiated state and is multipotent, capable of differentiation into multiple cell types. Adult stem cells can be derived from any adult, neonatal or fetal tissue such as adipose tissue, skin, kidney, liver, prostate, pancreas, intestine, bone marrow and placenta. Induced pluripotent stem cells or “iPSCs” may refer to any cells obtained by de-differentiation of adult somatic cells which are endowed with pluripotency, a cell being capable of differentiating into the three embryonic germ cell layers, the endoderm, ectoderm and mesoderm. Such cells may be obtained from a differentiated tissue (e.g. a somatic tissue such as skin) and undergo de-differentiation by genetic manipulation which reprogram the cell to acquire stem cell-like characteristics. iPSCs may be formed through a process that reverses the developmental potential of a cell or population of cells (e.g., a somatic cell). An iPSC may be a cell that has undergone a process of driving a cell to a state with higher developmental potential, such as a cell that is driven backwards to a less differentiated state. The somatic cell, prior to induction to an iPSC, can be either partially or terminally differentiated. There may be a complete or partial reversion of the differentiation state, e.g., an increase in the developmental potential of a cell, to that of a cell having a pluripotent state. A somatic cell may be driven to a pluripotent state, such that the cell has the developmental potential of an embryonic stem cell, similar to an embryonic stem cell phenotype. Induction of a somatic cell may also encompass a partial reversion of the differentiation state or a partial increase of the developmental potential of a cell, such as a somatic cell or a unipotent cell, to a multipotent state. Induction may also encompass partial reversion of the differentiation state of a cell to a state that renders the cell more susceptible to complete induction to a pluripotent state when subjected to additional manipulations. A stem cell may comprise a reprogrammed cell. Cellular reprogramming may be a process that reverses the developmental potential of a cell or population of cells (e.g., a somatic cell). Reprogramming may be a process of driving a cell to a state with higher developmental potential, such as driving a cell backwards to a less differentiated state. The cell to be reprogrammed can be either partially or terminally differentiated prior to reprogramming. Reprogramming may infer a complete or partial reversion of the differentiation state, such as an increase in the developmental potential of a cell, to that of a cell having a pluripotent state, driving a somatic cell to a pluripotent state, such that the cell has the developmental potential of an embryonic stem cell, such as an embryonic stem cell phenotype, or may encompass a partial reversion of the differentiation state or a partial increase of the developmental potential of a cell, such as a somatic cell or a unipotent cell, to a multipotent state. Reprogramming may also encompass a partial reversion of the differentiation state of a cell to a state that renders the cell more susceptible to complete reprogramming to a pluripotent state when subjected to additional manipulations. Hematopoietic stem cells may be adult tissue stem cells, including stem cells obtained from blood or bone marrow tissue of an individual at any age or from cord blood of a newborn individual. These cells may give rise to other blood cells during hematopoiesis. Hematopoietic stem cells may have the ability to self-renew and may be pluripotent, able to generate any and all diverse mature functional hematopoietic cell types such as erythrocytes, platelets, basophils, neutrophils, eosinophils, monocytes, T-lymphocytes, and B-lymphocytes. Mesenchymal stem cells may be multipotent stromal cells that can differentiate into a variety of cell types, including osteoblasts (bone cells), chondrocytes (cartilage cells), myocytes (muscle cells), adipocytes (fat cells which give rise to marrow adipose tissue), and neuron-like cells. A non-mammary adult stem cell may comprise a mesenchymal stem cell. Mesenchymal stem cells may be derived from the marrow as well as other non-marrow tissues, such as placenta, umbilical cord blood, adipose tissue, adult muscle, corneal stroma or the dental pulp of deciduous baby teeth. The cells may not have the capacity to reconstitute an entire organ but may be capable of self-renewal while maintaining their multipotency. A non-mammary adult stem cell may comprise a derivative of a non-mammary adult stem cell or a mesenchymal stem cell. A progenitor cell may comprise any cell that maintains the ability to differentiate into at least one specific type of cells but is more specified than a stem cell and pushed to differentiate to a target cell. Progenitor cells may not be able to replicate indefinitely and may divide a limited number of times. A cell may also comprise a reprogrammed cell such as a transdifferentiated mature cell wherein a somatic cell may be reprogrammed or otherwise induced into another lineage without going through an intermediary proliferative stem cell phase. Transdifferentiated mature cells may be somatic cells that are reprogrammed or otherwise induced into another lineage without going through an intermediate proliferative pluripotent stem cell stage. Direct transdifferentiation of mature cells may occur through transient, forced expression of transcription factors, different methods of transfection, culture conditions, and supplementation of small molecules or growth factors. Stem cells can be adult stem cells, induced pluripotent stem cells (iPSCs), or any other cell naturally capable of differentiating, or engineered to differentiate, into a mammary cell or a mammary-like cell. Stem cells can be differentiated into the mammary lineage by culturing in MammoCult media. Culturing the cells with estrogen and progesterone can help with lineage commitment to the mammary lineage. Mammary lineage cells can have an EpCAMhighCD14⁺CD29^{low}CD49b⁺CD49f^{low}CD61⁺Scal^{low}Promininl^{low}-ALDEFLUOR^{bright}phenotype, which can be used in the isolation and enrichment of these cells.

In some embodiments, stem cells may be obtained from mammalian adipose tissue, muscle tissue, cord blood, bone marrow, organ tissue, mammary tissue, extra-embryonic tissue, umbilical cord blood, tendon, periodontal ligament, synovial membrane, trabecular bone, bone marrow, nervous system, skin, periosteum, muscle, and body fluids, such as peripheral blood, or breastmilk, or a combination thereof, can be differentiated into mammary cells using known methods or the methods described herein. In an embodiment, cells can be isolated from such tissues types obtained from human; ungulate, including, but not limited to, bovine, including domestic dairy cow, bison, goat, sheep, buffalo, yak, horse, donkey, zebu, reindeer, deer, pig, peccarie, hippopotamus, camel, chevrotain, deer, giraffes, pronghorn, antelope, elk, moose, zebra, rhinoceros, and cattle, elephant; canine (family Canidae) including, but not limited to, domestic dog, coyote, fox, wolf, and dingo; feline (family Felidae) including, but not limited to, lion, tiger, snow leopard, jaguar, panther, mountain lion, domestic cat, bobcat, and lynx; aquatic mammals including, but not limited to, cetaceans such as whales, narwhals, dolphins, and porpoise; pinnipeds including, but not limited to, seals, walrus, and sea lions; marsupials including, but not limited to, kangaroo, wallaby, koala, phalangeriform, opossum, wombat, Tasmanian devil, dunnart, potoroo, and cuscus; rodent; bat; squirrel, panda, mouse, hedgehog, monkey, chimpanzee, ox, raccoon, horse, mole, armadillo, beaver, gibbon, gopher, Llama, orangutan, orca, otter, polar bear, porcupine, puma, rabbit, rat, and weasel.

In some embodiments, mammary epithelial cells, or mammary-like cells, can be isolated from specific mammary tissue, or such cells can be obtained from the differentiation of stem cells. Mammary cells or mammary-like cells can be purified and used in culture or can be transformed with a suitable gene to “immortalize” the cells, which may result in substantially indefinite cell division. For example, immortalized human mammary cells for oncological research have been made by directly targeting the two main senescence barriers encountered by cultured human mammary epithelial cells. The stress-associated stasis barrier may be bypassed using shRNA to p16^{INK4}; and replicative senescence due to critically shortened telomeres may be bypassed in post-stasis HMEC by c-MYC transduction. See, Garbe et al., Cell Cycle. 2014 Nov. 1; 13(21): 3423-3435, which is incorporated herein by reference in its entirety for all purposes. Another immortalized human mammary cell line may include hTERT-HME1. Huynh et al, may also describe an immortalized bovine mammary epithelial cell line, HH2A, for the study of mammary function and tissue. See, Huynh, et al., In Vitro Cellular & Developmental Biology—Animal, vol. 31, pages 25-29 (1995), which is incorporated herein by reference in its entirety for all purposes. Other immortalized mammary epithelial cell lines may be developed, such as those designated as MAC-T, CMEC-H, and BME-UV1. Still further, other mammalian mammary epithelial cell lines may be immortalized, including goat, that may be immortalized with human telomerase; see Shi et al, Animal Science Journal, Vol. 85:7, 2014, pages 735-743, which is incorporated herein by reference in its entirety for all purposes. Yak mammary epithelial cell line may be established by inserting the EGFP gene into the cells. See, Fu et al. (2014), entitled “Establishment of Mammary Gland Model In Vitro: Culture and Evaluation of a Yak Mammary Epithelial Cell Line,” PLoS ONE 9(12): e113669. doi: 10.1371/journal.pone.0113669, which is incorporated herein by reference in its entirety for all purposes.

In some embodiments, mammary epithelial cells, or mammary-like cells, can be isolated from specific mammary tissue, or such cells can be obtained from the differentiation of stem cells or progenitor cells. The mammary cells or mammary-like cells can be purified and used in culture or can be transformed with a suitable gene to “immortalize” the cells, which may result in substantially indefinite cell division. Immortalized human mammary cells for oncological research have been made by directly targeting the two main senescence barriers encountered by cultured human mammary epithelial cells. The stress-associated stasis barrier may be bypassed using shRNA to p16^{INK4}; and replicative senescence due to critically shortened telomeres may be bypassed in post-stasis HMEC by c-MYC transduction. See, Garbe et al., Cell Cycle. 2014 Nov. 1; 13(21): 3423-3435, which is incorporated herein by reference in its entirety for all purposes. Another immortalized human mammary cell line may include hTERT-HME1. Further, Huynh et al, describe an immortalized bovine mammary epithelial cell line, HH2A, for the study of mammary function and tissue. See, Huynh, et al., In Vitro Cellular & Developmental Biology—Animal, vol. 31, pages 25-29 (1995), which is incorporated herein by reference in its entirety for all purposes. Other immortalized mammary epithelial cell lines may have been developed, such as those designated as MAC-T, CMEC-H, and BME-UV1. Still further, other mammalian mammary epithelial cell lines may have been immortalized, including goat, that was immortalized with human telomerase; see Shi et al, Animal Science Journal, Vol. 85:7, 2014, pages 735-743, which is incorporated herein by reference in its entirety for all purposes. A yak mammary epithelial cell line may be established by inserting the EGFP gene into the cells. See, Fu M, Chen Y, Xiong X, Lan D, Li J (2014); “Establishment of Mammary Gland Model In Vitro: Culture and Evaluation of a Yak Mammary Epithelial Cell Line.” PLoS ONE 9(12): e113669. doi: 10.1371/journal.pone.0113669, which is incorporated herein by reference in its entirety for all purposes.

### Cell Culture Media

Aspects of the present disclosure may comprise a cell culture medium. A cell culture medium may comprise a differentiation medium, a growth medium, or a lactogenic medium. A lactogenic medium may comprise a hormone or a signaling factor. A signaling factor may comprise a growth factor or a growth hormone such as glucocorticoid, insulin, progesterone, prolactin, or estrogen. A medium may comprise a reagent capable of regulating or enhancing a cellular response to a hormone or signaling factor. A reagent capable or regulating or enhancing a cellular response to a hormone or a signaling factor may comprise a small molecule, a small interfering ribonucleotide (siRNA), a peptide, a nucleic acid, or a transcription factor. A reagent may be capable of activating the expression of a hormone or a signaling factor in the cell. Mammary cells, such as bovine mammary cells may be cultured according to standard mammalian cell culture conditions. For example, but not limited to: the growth media can include DMEM/F12 and about 10% fetal bovine serum (FBS) (available from Invitrogen). Mammary or mammary-like cells can be cultured in standard mammalian cell culture media, and can include the use of DMEM-F12, RPMI, and Alpha-MEM supplemented with fetal bovine serum, and antibiotics.

Isolated cells may be grown and cultured in 2D and/or 3D culture systems. The cells may be differentiated into mammary epithelial and mammary luminal cells and induced to lactate. Mammary cells can be induced to lactate when exposed to certain environmental conditions, including various hormones, proteins, and stimulants and/or temperature levels. Lactation media, which may promote the synthesis of milk components, such as protein, oligosaccharides, and fat, can include a growth medium supplemented with about 5 mg/mL insulin, about 5 mg/mL Holotransferrin, about 5 mg/mL progesterone, about 1027 mol/L hydrocortisone, about 10 ng/mL epithelial growth factor and about 5 mg/mL bovine estradiol (Sigma-Aldrich, cat. #14434, T1283, P8783, H0888, E4127, E2758, respectively). The mammary or mammary-like cells can be induced to lactation using a lactation medium including using at least one bioactive compound, such as prolactin, insulin, a glucocorticoid, PQQ, etc. See, for example, Mitchell et al., entitled “Characterization of pyrroloquinoline quinone amino acid derivatives by electrospray ionization mass spectrometry and detection in human milk,” Anal Biochem. 1999; 269(2):317-325; doi:10.1006/abio.1999.4039, which is incorporated herein by reference in its entirety for all purposes. Upon induction of lactation, the cells secrete milk components into the medium, and the components can be isolated as the liquid composition described herein, by filtration to remove cellular debris, dialysis to remove and equilibrate metabolic waste products and other undesired waste products, resin purification to capture and remove materials that are not desirable in the product.

A culture of mammary cells, or mammary-like cells, may be subjected to 2D or 3D culture conditions to grow and maintain the cells and to induce the production of the nutrient composition (such as liquid nutrient composition). As provided herein, three-dimensional (3D) scaffolding and tissue engineering platforms may be used to facilitate large-scale growth. A scaffold may provide structural support and guide the growth of the cultured cells into the desired structure and/or texture analogous with the equivalent food composition or food product produced using conventional methods. Cell or tissue culture may comprise growing the population of cells on scaffolds within a bioreactor.

A scaffold may enable cell adhesion in a cell culture. A scaffold may enable adherent cells to be grown in a bioreactor system. A bioreactor system may be adherent or a suspension bioreactor system. Culturing stem cells in contact with a scaffold may be performed in a bioreactor chamber and subjecting at least a subset of the cultured stem cells to one or more expansion processes may be performed in a same bioreactor chamber. As used herein, the term “expansion” generally refers to growing a population of cells exponentially into larger systems. Cellular expansion is a process that results in an increase of the number of cells and is defined by the balance between cell divisions and cell loss through death or differentiation. Culturing stem cells in contact with a scaffold may be performed in a bioreactor chamber and subjecting at least a subset of the cultured stem cells to one or more expansion processes may be performed in an additional bioreactor chamber. One or more of cell culturing, expansion, or differentiation processes may be performed in a same bioreactor chamber, or each may be conducted in a different bioreactor chamber. In some cases, cell culturing may be performed in a bioreactor camber and cell expansion performed in a different bioreactor chamber.

Cultured cells may receive some degree of structural integrity from a scaffold on which the cells may be attached during culturing. Alternatively, a scaffold may not be necessary in suspended cell cultures. Non-adherent cells may not require a substrate or surface for attachment. Cells may have been modified or engineered to no longer require an adherence substrate. For example, hepatocytes are normally adherent cells, but may be modified to no longer require an extracellular matrix for attachment for survival and proliferation. Cultured cells may be grown into cultured tissues that are attached to a support structure such as a two-dimensional or three-dimensional scaffold or support structure. A scaffold or support structure may be degradable. Cultured cells may be grown on a two-dimensional support structure such as a petri-dish where they may form several layers of cells that may be peeled and processed for consumption. Two-dimensional support structures may include porous membranes that allow for diffusion of nutrients from culture media on one side of the membrane to the other side where the cells are attached. In such a composition, additional layers of cells may be achieved by exposing the cells to culture media from both sides of the membrane, for example, cells may receive nutrients through diffusion from one side of the membrane and also from the culture media covering the cells growing on the membrane.

Cultured cells may be grown on, around, or inside a three-dimensional support structure. The support structure may be sculpted into different sizes, shapes, and forms to provide the shape and form for the cultured cells to grow. The support structure may be a natural or synthetic biomaterial. A biomaterial may comprise any substance intended to interface with biological systems to evaluate, treat, augment, or replace any tissue, organ, or function in a biocompatible manner, such as with a level of acceptable biological response. A biomaterial may interact passively with cells and tissues or may comprise a bioactive material which induces a specific and intended biological response. A biomaterial may comprise a substrate that has been engineered to take a form which alone or as part of a complex system, is used to direct, by control of interactions with components of living systems. A biomaterial may be natural, synthetic, or some combination thereof. A scaffold may be composed of one material or one or more different materials. The support structure may be non-toxic and edible so that they may not be harmful if ingested and may provide additional nutrition, texture, flavor, or form to the food composition or food product. A scaffold may comprise a hydrogel, a biomaterial such as an extracellular matrix molecule (ECM), or biocompatible synthetic material. ECM molecules may comprise proteoglycans, non-proteoglycan polysaccharides, or proteins. A micro-scaffold may be smaller than a conventional tissue culture scaffold which may provide a macroscopic structure and/or shape for the cell population. A micro-scaffold may provide a surface for adherent cells to attach to even while the micro-scaffold itself is in suspension. A micro-scaffold may provide a seed or core structure for adherent cells to attach while remaining small enough to remain in suspension with stirring. The use of micro-scaffolds enables the culturing of adherent cells in a suspension culture which may enable the large-scale production of adherent cells. An edible dairy product may be generated using the tissue produced and a degradable scaffold. As an example, the scaffold may be used to guide (as a framework) or facilitate the production the dairy product.

A degradable scaffold may comprise a polymeric material. A polymeric material may comprise a natural polymeric material or a synthetic polymeric material. Natural biomaterials may comprise collagen, gelatin, fibrin, alginate, agar, cassava, maize, chitosan, gellan gum, corn-starch, chitin, cellulose, chia (Salvia hispanica) recombinant silk, decellularized tissue (plant or animal), hyaluronic acid, fibronectin, laminin, hemicellulose, glucomannan, textured vegetable protein, heparan sulfate, chondroitin sulfate, tempeh, keratan sulfate, or any combination thereof. A plant-based scaffold may be used for 3D culturing. A plant-based scaffold may comprise scaffolds obtained from plants such as apples, seaweed, or jackfruit. A plant-based scaffold may comprise at least one plant-based material such as cellulose, hemicellulose, pectin, lignin, alginate, or any combination thereof. A textured vegetable protein (TVP), such as textured soy protein (TSP) may comprise a high percentage of soy protein, soy flour, or soy concentrate. TVP and TSP can be used to provide a meat-like texture and consistency to a meat product. Synthetic biomaterials may comprise hydroxyapatite, polyethylene terephthalate, acrylates, polyethylene glycol, polyglycolic acid, polycaprolactone, polylactic acid, their copolymers, or any combination thereof.

A support structure may include adhesion peptides, cell adhesion molecules, or other growth factors covalently or non-covalently associated with the support structure. Cell recognition sites may promote cell adhesion and migration. Cell recognition sites may comprise sequences such as Arg-Gly-Asp (RGD) or Arg-Glu-Asp-Val sequences. A synthetic polymeric material may comprise a polyethylene glycol biomaterial comprising an arginylglycylaspartic (RGD) motif. A dairy product comprising scaffolding material may be seasoned to taste like a dairy product (e.g., using various salts, herbs, and/or spices). A scaffold may be comprised of a cell or tissue culture product.

A support structure may be formed as a solid or semisolid support. A support structure may comprise a solid non-porous structure or a porous structure, for example, high porosity may provide maximal surface area for cell attachment. Porous scaffolds may allow cell migration or infiltration into the pores. A porous scaffold may be edible. A porous scaffold may comprise a natural biomaterial or a synthetic biomaterial, textured protein. A porous scaffold may have an average pore diameter. An average pore diameter of the porous scaffold may range from about 20 micrometers (μm) to about 1000 μm, 20 μm to 900 μm, 20 μm to 800 μm, 20 μm to 700 μm, 20 μm to 600 μm, 20 μm to 500 μm, 20 μm to 400 μm, 20 μm to 300 μm, 20 μm to 200 μm, 20 μm to 100 μm, 50 μm to 1000 μm, 50 μm to 900 μm, 50 μm to 800 μm, 50 μm to 700 μm, 50 μm to 600 μm, 50 μm to 500 μm, 50 μm to 400 μm, 50 μm to 300 μm, 50 μm to 200 μm, 50 μm to 100 μm, 100 μm to 1000 μm, 100 μm to 900 μm, 100 μm to 800 μm, 100 μm to 700 μm, 100 μm to 600 μm, 100 μm to 500 μm, 100 μm to 400 μm, 100 μm to 300 μm, 100 μm to 200 μm, 500 μm to 1000 μm, 500 μm to 900 μm, 500 μm to 800 μm, 500 μm to 700 μm, or 500 μm to 600 μm. An average pore diameter of the porous scaffold may range from about 20 μm to about 1000 μm. An average pore diameter may be less than 20 μm or may be larger than 1000 μm. A soft, porous material may be selected with an adequate microstructure and stiffness for the cell type of interest. A scaffold may confer mechanical properties to improve the texture and mouthfeel of a dairy product. A scaffold may also confer mechanical properties to encourage proliferation, migration, growth, or differentiation of a desired cell type from a precursor cell. A mechanical property may comprise compression, expansion, strain, stretch, elasticity, shear strength, shear modulus, viscoelasticity, or tensile strength. A scaffold may comprise a material with suitable mechanical properties and degradation kinetics for the desired tissue type that is generated from the cells. For example, a softer surface may be needed in the differentiation and culture of adipocytes as compared to ductal cells.

A scaffold may be produced by transforming a material. A scaffold fabrication method may comprise a physical and/or chemical performed on a material to render them usable for cell or tissue culture. Not all biomaterials may be suitable for a given fabrication method or a biomaterial may need to be modified to enable their use in a fabrication method. A scaffold fabrication method may comprise electrospinning, phase separation, freeze drying, lithography, printing, extrusion, self-assembly, solvent casting, textile technologies, material injections, laser sintering, phase separation, porogen leaching, gas foaming, fiber meshing, supercritical fluid processing, or additive manufacturing.

A support structure may comprise a degradable scaffold. A degradable scaffold may be configured to facilitate cell expansion in a culture vessel, such as a bioreactor chamber. As used herein, the term “bioreactor” generally refers to any manufactured device or system which supports a biologically active environment. A bioreactor may refer to a container suitable for the cultivation of eukaryotic cells, such as mammalian animal cells, or tissues in the context of cell culture. A bioreactor may culture various cell types together, in parallel, or may culture one cell type singularly. A bioreactor may comprise one vessel or a plurality of vessels and may recycle media used during culture. A degradable scaffold may be configured to facilitate cell expansion inside a bioreactor chamber. Stem cells may be cultured in the presence of a degradable scaffold to create cultured stem cells. Stem cells may be cultured into cultured stem cells and cultured stem cells may be subjected to one or more expansion processes to generate expanded stem cells in the presence of a degradable scaffold. In some embodiments, a bioreactor vessel can comprise a spinning stir rod to mix the media. Stem cells are seeded on scaffolding placed in the bioreactor vessel. A degradable scaffold may degrade at approximately an equal rate to tissue formation. A degradable material may enable remodeling and/or elimination of the scaffold in the cultured food composition or food product. The scaffold may also comprise a material that remains in the cultured food composition or food product. For example, a portion of a collagen scaffold providing support to cultured myoepithelial cells may remain in a desired dairy product to provide texture and continuing structural support in the cultured food composition or food product. A scaffold may comprise materials that do not biodegrade and/or remain in the cultured food composition or food product for consumption. For example, certain materials can be used to generate the scaffolds in order to confer a particular structure, texture, taste, or other desired property without degradation. A scaffold may comprise a material with texture-modifying properties.

Scaffolds of various compositions can be used to produce a desired texture and/or consistency in the desired food composition or food product. A natural biomaterial such as a gellan gum, corn starch, chia, or cassava material may produce a desired texture, consistency, or flavor profile to a food composition or food product. A scaffold may comprise a filler or binder material for providing texture to the food composition or food product or may be a filler or binder material for providing texture to a food composition or food product. A scaffold material may biodegrade such that the finished food composition or food product no longer has any scaffold structures remaining. For example, a population of cells may be seeded onto a scaffold in a bioreactor. As the cells adhere to the scaffolds and proliferate, the scaffolds gradually biodegrade until all that remains are the clumps of cells that are now adhered to each other and the extracellular matrix materials that they have secreted. A scaffold can be used to guide the structure of the resulting cultured food composition or food product and may remain in the food composition or food product for consumption by a human. For example, a scaffold for the proliferation of mammary epithelial cells may comprise a gellan gum material. This material may be engineered such that it partially biodegrades by the time a dairy product is produced in culture. The gellan gum may remain in the dairy product acting as a filler and as a texture and flavor enhancer.

Provided herein are systems and methods for producing cultured dairy for food consumption. An edible food composition or food product may be derived from the expansion and differentiation of cells, such as the expansion and differentiation of stem cells or progenitor cells. Expansion may comprise growing a population of cells exponentially into larger systems. Cellular expansion may be a process that results in an increase of the number of cells and may be affected by the balance between cell divisions and cell loss through death or differentiation. A cell may be grown in a high-density cell culture.

Culture conditions and the associated growth, maintenance, or induction media can be carried out in any reaction vessel useful for culturing mammalian cells. Cells may be cultured and expanded to a desired quantity such as in a scalable manner using bioreactors to enable large-scale production. A bioreactor apparatus may provide a scalable method for differentiating and expanding stem cells into tissue and with the requisite growth needed for industrial production. Further, the mechanical conditioning of such an apparatus may provide a uniform method of producing a bio-artificial dairy product that simulates standard dairy in terms of its appearance, texture, and flavor at a competitive price. Suitable reaction vessels can be continuous flow, or batch. By way of example, suitable reaction vessels include, but are not limited to fermentation bioreactors, stirred tank bioreactors, tidal bioreactors, bello-cell bioreactors, roller bottle bioreactors, packed bed bioreactors, bubbled column bioreactors, airlift bioreactors, tower bioreactors, and fluidized bed bioreactors.

A bioreactor system may comprise at least one bioreactor, bioreactor tank, or reactor chamber. For example, a bioreactor system may comprise at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 reactor chambers. A bioreactor system may comprise about 1 reactor chamber to more than 1,000 reactor chambers. A bioreactor system may comprise about 1 reactor chamber or more than 1 reactor chambers. A reactor chamber may have an internal volume suitable for large-scale cell culture. A reactor chamber may have an internal volume of about 0.1 Liters (L) to about 1,000,000 L. A reactor chamber may have an internal volume of about less than 1 L or an internal volume of greater than about 1,000,000 L. A reactor chamber may have an internal volume of at least about 1 L to about 10 L, about 1 L to about 50 L, about 1 L to about 100 L, about 1 L to about 500 L, about 1 L to about 1,000 L, about 1 L to about 5,000 L, about 1 L to about 10,000 L, about 1 L to about 50,000 L, about 1 L to about 1,000,000 L, about 10 L to about 50 L, about 10 L to about 100 L, about 10 L to about 500 L, about 10 L to about 1,000 L, about 10 L to about 5,000 L, about 10 L to about 10,000 L, about 10 L to about 50,000 L, about 10 L to about 1,000,000 L, about 50 L to about 100 L, about 50 L to about 500 L, about 50 L to about 1,000 L, about 50 L to about 5,000 L, about 50 L to about 10,000 L, about 50 L to about 50,000 L, about 50 L to about 1,000,000 L, about 100 L to about 500 L, about 100 L to about 1,000 L, about 100 L to about 5,000 L, about 100 L to about 10,000 L, about 100 L to about 50,000 L, about 100 L to about 1,000,000 L, about 500 L to about 1,000 L, about 500 L to about 5,000 L, about 500 L to about 10,000 L, about 500 L to about 50,000 L, about 500 L to about 1,000,000 L, about 1,000 L to about 5,000 L, about 1,000 L to about 10,000 L, about 1,000 L to about 50,000 L, about 1,000 L to about 1,000,000 L, about 5,000 L to about 10,000 L, about 5,000 L to about 50,000 L, about 5,000 L to about 1,000,000 L, about 10,000 L to about 50,000 L, about 10,000 L to about 1,000,000 L, or about 50,000 L to about 100,000 L or more than about 1,000,000 L.

Cell culturing, differentiation and/or expansion may each be conducted in a separate bioreactor chamber. In some examples, all processes (e.g., culturing, expansion, differentiation) may be performed in the same bioreactor chamber. As another example, cell culturing may be performed in a bioreactor chamber and expansion and/or differentiation may be performed in an additional bioreactor chamber. The bioreactor chamber or the additional bioreactor chamber may comprise a plurality of bioreactor chambers. Each of the plurality of the bioreactor chambers or the additional bioreactor chambers may be configured to facilitate a specific process (e.g., culturing, expansion, differentiation). In some cases, a subset or all of the cultured stem cells from the bioreactor chamber may be directed to a plurality of additional bioreactor chambers to perform a plurality of expansion processes, which may comprise greater than or equal to about 1, 2, 3, 4, 5, 6, 7, 8, 9, 10, 15, 20, 25, 30, 35, 40, 45, 50 expansion processes, or more. The plurality of expansion processes may be performed sequentially, simultaneously, or a combination thereof.

A bioreactor system may be suitable for large-scale production of cultured cells for generation of food compositions or food products. Cells may be cultured on a batch basis. Alternatively, or in combination, cells may be cultured continuously. In both batch and continuous cultures, fresh nutrients may be supplied to ensure the appropriate nutrient concentrations for producing the desired food composition or food product. As an example, in a fed-batch culture, nutrients (e.g. fresh culture media) is supplied to the bioreactor, and the cultured cells remain in the bioreactor until they are ready for processing into the finished food composition or food product. In a semi-batch culture, a base media may be supplied to the bioreactor and may support an initial cell culture, while an additional feed media is then supplied to replenish depleted nutrients. A bioreactor system may produce at least a certain quantity of cells per batch. A bioreactor system may produce a batch of about 1 billion cells to about 100,000,000 billion cells. A bioreactor system may produce a batch of at least about 1 billion cells. A bioreactor system may produce a batch of about 100,000,000 billion cells. A bioreactor system may produce a batch of less than 1 billion cells. A bioreactor system may produce a batch of at least about 1 billion cells, about 1 billion cells to 10 billion cells, about 1 billion cells to about 50 billion cells, about 1 billion cells to about 100 billion cells, about 1 billion cells to about 500 billion cells, about 1 billion cells to about 1,000 billion cells, about 1 billion cells to about 5,000 billion cells, about 1 billion cells to about 10,000 billion cells, about 1 billion cells to about 100,000 billion cells, about 1 billion cells to about 1,000,000 billion cells, about 1 billion cells to about 10,000,000 billion cells, about 1 billion cells to about 100,000,000 billion cells, about 10 billion cells to about 50 billion cells, about 10 billion cells to about 100 billion cells, about 10 billion cells to about 500 billion cells, about 10 billion cells to about 1,000 billion cells, about 10 billion cells to about 5,000 billion cells, about 10 billion cells to about 10,000 billion cells, about 10 billion cells to about 100,000 billion cells, about 10 billion cells to about 1,000,000 billion cells, about 10 billion cells to about 10,000,000 billion cells, about 10 billion cells to about 100,000,000 billion cells, about 50 billion cells to about 100 billion cells, about 50 billion cells to about 500 billion cells, about 50 billion cells to about 1,000 billion cells, about 50 billion cells to about 5,000 billion cells, about 50 billion cells to about 10,000 billion cells, about 50 billion cells to about 100,000 billion cells, about 50 billion cells to about 1,000,000 billion cells, about 50 billion cells to about 10,000,000 billion cells, about 50 billion cells to about 100,000,000 billion cells, about 100 billion cells to about 500 billion cells, about 100 billion cells to about 1,000 billion cells, about 100 billion cells to about 5,000 billion cells, about 100 billion cells to about 10,000 billion cells, about 100 billion cells to about 100,000 billion cells, about 100 billion cells to about 1,000,000 billion cells, about 100 billion cells to about 10,000,000 billion cells, about 100 billion cells to about 100,000,000 billion cells, about 500 billion cells to about 1,000 billion cells, about 500 billion cells to about 5,000 billion cells, about 500 billion cells to about 10,000 billion cells, about 500 billion cells to about 100,000 billion cells, about 500 billion cells to about 1,000,000 billion cells, about 500 billion cells to about 10,000,000 billion cells, about 500 billion cells to about 100,000,000 billion cells, about 1,000 billion cells to about 5,000 billion cells, about 1,000 billion cells to about 10,000 billion cells, about 1,000 billion cells to about 100,000 billion cells, about 1,000 billion cells to about 1,000,000 billion cells, about 1,000 billion cells to about 10,000,000 billion cells, about 1,000 billion cells to about 100,000,000 billion cells, about 5,000 billion cells to about 10,000 billion cells, about 5,000 billion cells to about 100,000 billion cells, about 5,000 billion cells to about 1,000,000 billion cells, about 5,000 billion cells to about 10,000,000 billion cells, about 5,000 billion cells to about 100,000,000 billion cells, about 10,000 billion cells to about 100,000 billion cells, about 10,000 billion cells to about 1,000,000 billion cells, about 10,000 billion cells to about 10,000,000 billion cells, about 10,000 billion cells to about 100,000,000 billion cells, about 100,000 billion cells to about 1,000,000 billion cells, about 100,000 billion cells to about 10,000,000 billion cells, about 100,000 billion cells to about 100,000,000 billion cells, about 1,000,000 billion cells to about 10,000,000 billion cells, about 1,000,000 billion cells to about 100,000,000 billion cells, or about 10,000,000 billion cells to about 100,000,000 billion cells or more than 100,000,000 billion cells.

A bioreactor system may produce a batch of cultured cells during a certain time period. For example, in some cases, a bioreactor system may produce a batch of cultured cells at least once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, or more. A bioreactor system may produce a batch of cultured cells having at least a certain mass. Sometimes, the mass is measured as dry weight with excess media or supernatant removed. A bioreactor system may produce a batch of cultured cells of about 1 kilogram (kg) to about 100,000 kg. In certain instances, a bioreactor system may produce a batch of cultured cells of less than 1 kg. A bioreactor system may produce a batch of about 100,000 kg or more than 100,000 kg. A bioreactor system may produce a batch of about less than 1 kg to 1 kg, about 1 kg to about 5 kg, about 1 kg to about 10 kg, about 1 kg to about 20 kg, about 1 kg to about 30 kg, about 1 kg to about 40 kg, about 1 kg to about 50 kg, about 1 kg to about 100 kg, about 1 kg to about 500 kg, about 1 kg to about 1,000 kg, about 1 kg to about 5,000 kg, about 1 kg to about 100,000 kg, about 5 kg to about 10 kg, about 5 kg to about 20 kg, about 5 kg to about 30 kg, about 5 kg to about 40 kg, about 5 kg to about 50 kg, about 5 kg to about 100 kg, about 5 kg to about 500 kg, about 5 kg to about 1,000 kg, about 5 kg to about 5,000 kg, about 5 kg to about 100,000 kg, about 10 kg to about 20 kg, about 10 kg to about 30 kg, about 10 kg to about 40 kg, about 10 kg to about 50 kg, about 10 kg to about 100 kg, about 10 kg to about 500 kg, about 10 kg to about 1,000 kg, about 10 kg to about 5,000 kg, about 10 kg to about 100,000 kg, about 20 kg to about 30 kg, about 20 kg to about 40 kg, about 20 kg to about 50 kg, about 20 kg to about 100 kg, about 20 kg to about 500 kg, about 20 kg to about 1,000 kg, about 20 kg to about 5,000 kg, about 20 kg to about 100,000 kg, about 30 kg to about 40 kg, about 30 kg to about 50 kg, about 30 kg to about 100 kg, about 30 kg to about 500 kg, about 30 kg to about 1,000 kg, about 30 kg to about 5,000 kg, about 30 kg to about 100,000 kg, about 40 kg to about 50 kg, about 40 kg to about 100 kg, about 40 kg to about 500 kg, about 40 kg to about 1,000 kg, about 40 kg to about 5,000 kg, about 40 kg to about 100,000 kg, about 50 kg to about 100 kg, about 50 kg to about 500 kg, about 50 kg to about 1,000 kg, about 50 kg to about 5,000 kg, about 50 kg to about 100,000 kg, about 100 kg to about 500 kg, about 100 kg to about 1,000 kg, about 100 kg to about 5,000 kg, about 100 kg to about 100,000 kg, about 500 kg to about 1,000 kg, about 500 kg to about 5,000 kg, about 500 kg to about 100,000 kg, about 1,000 kg to about 5,000 kg, about 1,000 kg to about 100,000 kg, or about 5,000 kg to about 100,000 kg or more than 100,000 kg.

Cell and tissue culture may occur in one or a plurality of bioreactors or bioreactor chambers throughout growth, expansion, and differentiation. There may be 1, 2, 3, 4, 5, 6, 7, 8, 9, 10, or more than 10 bioreactors or bioreactor chambers used in cell or tissue culture. In some embodiments, a bioreactor system may comprise about 1 reactor chamber to about 5 reactor chambers, about 1 reactor chamber to about 10 reactor chambers, about 1 reactor chamber to about 20 reactor chambers, about 1 reactor chamber to about 50 reactor chambers, about 1 reactor chamber to about 100 reactor chambers, about 1 reactor chamber to about 200 reactor chambers, about 1 reactor chamber to about 300 reactor chambers, about 1 reactor chamber to about 400 reactor chambers, about 1 reactor chamber to about 500 reactor chambers, about 1 reactor chamber to about 1,000 reactor chambers, about 5 reactor chambers to about 10 reactor chambers, about 5 reactor chambers to about 20 reactor chambers, about 5 reactor chambers to about 50 reactor chambers, about 5 reactor chambers to about 100 reactor chambers, about 5 reactor chambers to about 200 reactor chambers, about 5 reactor chambers to about 300 reactor chambers, about 5 reactor chambers to about 400 reactor chambers, about 5 reactor chambers to about 500 reactor chambers, about 5 reactor chambers to about 1,000 reactor chambers, about 10 reactor chambers to about 20 reactor chambers, about 10 reactor chambers to about 50 reactor chambers, about 10 reactor chambers to about 100 reactor chambers, about 10 reactor chambers to about 200 reactor chambers, about 10 reactor chambers to about 300 reactor chambers, about 10 reactor chambers to about 400 reactor chambers, about 10 reactor chambers to about 500 reactor chambers, about 10 reactor chambers to about 1,000 reactor chambers, about 20 reactor chambers to about 50 reactor chambers, about 20 reactor chambers to about 100 reactor chambers, about 20 reactor chambers to about 200 reactor chambers, about 20 reactor chambers to about 300 reactor chambers, about 20 reactor chambers to about 400 reactor chambers, about 20 reactor chambers to about 500 reactor chambers, about 20 reactor chambers to about 1,000 reactor chambers, about 50 reactor chambers to about 100 reactor chambers, about 50 reactor chambers to about 200 reactor chambers, about 50 reactor chambers to about 300 reactor chambers, about 50 reactor chambers to about 400 reactor chambers, about 50 reactor chambers to about 500 reactor chambers, about 50 reactor chambers to about 1,000 reactor chambers, about 100 reactor chambers to about 200 reactor chambers, about 100 reactor chambers to about 300 reactor chambers, about 100 reactor chambers to about 400 reactor chambers, about 100 reactor chambers to about 500 reactor chambers, about 100 reactor chambers to about 1,000 reactor chambers, about 200 reactor chambers to about 300 reactor chambers, about 200 reactor chambers to about 400 reactor chambers, about 200 reactor chambers to about 500 reactor chambers, about 200 reactor chambers to about 1,000 reactor chambers, about 300 reactor chambers to about 400 reactor chambers, about 300 reactor chambers to about 500 reactor chambers, about 300 reactor chambers to about 1,000 reactor chambers, about 400 reactor chambers to about 500 reactor chambers, about 400 reactor chambers to about 1,000 reactor chambers, or about 500 reactor chambers to about 1,000 reactor chambers or more than 1,000 reactor chambers.

Growth, culturing, expansion, and differentiation may be concurrent or in parallel in the same or in different bioreactors or bioreactor chambers. For example, a bioreactor system may be designed such that there are two bioreactors in which iPSC expansion occurs and four bioreactors in which iPSC differentiation occurs. Cells may be grown within a first bioreactor of scalable size for a period of approximately 7 days. Cells may be grown for approximately, 1, 2, 3, 4, 5, 6, 7, 8, 9, 10, 15, 20, 25, 30, 35, 40, 45, 50, 60, 70, 80, 90, or more than 90 days. One or more expansion processes may comprise passaging at least a subset or all cultured stem cells. Cells may be passaged to a subsequent bioreactor approximately four times the size of the first bioreactor of scalable size. A subsequent bioreactor may be 2, 3, 4, 5, 6, 7, 8, 9, 10, or more than 10 times the size of the first bioreactor of scalable size. A subsequent bioreactor may be less than 10, 9, 8, 7, 6, 5, 4, 3, 2, or 1 time(s) the size of the first bioreactor of scalable size. Cultured cells may be “split” or “passaged” approximately every 7 days, but the cells can be split more often or less often, depending on the specific needs and circumstances of the culture. For example, the cells may be split every 1, 2, 3, 4, 5, 6, 7, 8, 9, 10, 11, 12, 13, 14 or more days, or any time frame in between. The cell split or passaging may comprise the collection of cells from a (e.g., previous) culture and subsequent transfer of the collected (harvested) cells into a new cell culture vessel. Passaging may allow the cells to continue to grow in a healthy cell culture environment. Processes and methods of cell culture passaging may involve the use of enzymatic or non-enzymatic methods to disaggregate cells that have clumped together during their growth expansion. Passaging may comprise passing an enzyme over at a subset or all cultured stem cells to detach them from a surface of the degradable scaffold. Cells can be passaged using enzymatic, non-enzymatic, or manual dissociation methods prior to and/or after contact with the defined medium. Non-limiting examples of enzymatic dissociation methods include the use of proteases such as trypsin, TrypLE, collagenase, dispase, and accutase. When enzymatic passaging methods are used, the resultant culture can comprise a mixture of singlets, doublets, triplets, and clumps of cells that vary in size depending on the enzymatic method used. A non-limiting example of a non-enzymatic dissociation method is a cell dispersal buffer or ethylenediaminetetraacetic acid (EDTA). The choice of passaging method may be influenced by the choice of cell type, extracellular matrix or a biomaterial scaffold, if one is present.

To passage cells from one bioreactor to the next, media may be drained from the bioreactor shelves and may be replaced by phosphate buffered saline (PBS) to wash the cells. PBS may be run over the cells such that each shelf in the bioreactor may be submerged in PBS for at least 15 seconds, after which the PBS may be removed and discarded. Each shelf in the bioreactor may be submerged in PBS for about at least 1 second (s), 2 s, 3 s, 4 s, 5 s, 6 s, 7 s, 8 s, 9 s, 10 s, 15 s, 20 s, 25 s, 30 s, 35 s, 40 s, 45 s, 50 s, 60 s, 70 s, 80 s, 90 s or more than 90 s. Each shelf in the bioreactor may be submerged for less than about 1 s. An enzyme or chemical solution such as EDTA in PBS may be passed over the cells to detach the cells from their surface of adhesion, for example a shelf, scaffold, or surface in the bioreactor. The cells may be incubated in the enzyme or chemical solution for a period of time, such as 4-8 minutes (min), before the solution is removed and discarded. The cells may be incubated in the enzyme or chemical solution for about at least 1 minute (min.)-2 min., 1 min.-3 min., 1 min.-4 min., 1 min.-5 min., 1 min.-6 min., 1 min.-7 min., 1 min.-8 min., 1 min.-9 min., 1 min.-10 min., or 1 min.-more than 10 min., 2 min.-3 min., 2 min.-4 min., 2 min.-5 min., 2 min.-6 min., 2 min.-7 min., 2 min.-8 min., 2 min.-9 min., 2 min.-10 min., 2 min.-more than 10 min., 3 min.-4 min., 3 min.-5 min., 3 min.-6 min., 3 min.-7 min., 3 min.-8 min., 3 min.-9 min., 3 min.-10 min., 3 min.-more than 10 min., 4 min.-5 min., 4 min.-6 min., 4 min.-7 min., 4 min.-8 min., 4 min.-9 min., 4 min.-10 min., 4 min.-more than 10 min., 5 min.-6 min., 5 min.-7 min., 5 min.-8 min., 5 min.-9 min., 5 min.-10 min., 5 min.-more than 10 min., 6 min.-7 min., 6 min.-8 min., 6 min.-9 min., 6 min.-10 min., 6 min.-more than 10 min., 7 min.-8 min., 7 min.-9 min., 7 min.-10 min., 7 min.-more than 10 min., 8 min.-9 min., 8 min.-10 min., 8 min.-more than 10 min., 9 min.-10 min., or 9 min.-more than 10 min. Cells may be incubated in an enzyme or chemical solution for less than 1 min or more than 10 min. Media from a media storage tank may be used to collect the detached cells by passing media over the cells and the cells in the media, may be collected in an additional tank to be passed to a centrifuge/cell filter system to isolate the cell and colony pieces from the media. A condensed cell/media solution may then be further mixed with media from a media storage tank as it flows into a subsequent bioreactor using decreasing flow rates to enable equal coating of bioreactor shelves. Cells may be separated using centrifugation or through an alternative method such as cell filtration which may separate cells of the size of a cell of interest out, such as an iPSC.

Cells may be expanded in a subsequent bioreactor for approximately 7 days or may be expanded in a same bioreactor for approximately 7 days. Cells may be expanded for approximately, 1, 2, 3, 4, 5, 6, 7, 8, 9, 10, 15, 20, 25, 30, 35, 40, 45, 50, 60, 70, 80, 90, or more than 90 days. Cells may be further passaged into one or a plurality of bioreactors which may be 2, 3, 4, 5, 6, 7, 8, 9, 10, or more than 10 times the size of a (e.g., previous) bioreactor of scalable size. A subsequent bioreactor may be less than 10, 9, 8, 7, 6, 5, 4, 3, 2, or 1 time the size of the (e.g., previous) bioreactor of scalable size thus splitting the cells by a ratio dependent on the size of the bioreactors and resultant density of the cultured cells.

Differentiation may occur in any one or combination of the bioreactors. Differentiation of a stem cell or progenitor cell into a terminally differentiated cell may take approximately 14-21 days or more. Differentiation of a stem cell or progenitor cell into a terminally differentiated cell may take 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or more than 90 days. Differentiation of a stem cell or progenitor cell into a terminally differentiated cell may take less than 90, 80, 70, 60, 50, 45, 40, 35, 30, 25, 20, 19, 18, 17, 16, 15, 14, 13, 12, 11, 10, 9, 8, 7, 6, 5, 4, 3, 2, 1, or less than one day.

Expansion and differentiation phases may use one or different types of media. Media and growth conditions may be optimized using different media, temperatures, conditions, or compositions. One or multiple media storage tanks may be used to store one or multiple types of media. Media storage tanks may comprise an area for storage of differentiation factors or small molecules in solution. Media storage tanks may be temperature controlled and individual tanks in a plurality of tanks may store media at different temperatures. For example, media may be stored at 4° C. and differentiation factors to be mixed with media stored at −20° C. Differentiation factors, media components, or media stored at freezing or below freezing temperatures may be thawed automatically and added into an appropriate media storage tank when required. Some media components may remain fresh for several weeks while some differentiation factors or nucleotides may be maintained as frozen as they may degrade rapidly in less than 24 hours. Media may comprise a serum or may utilize a serum free media. Culture medium may comprise maintenance media, differentiation media, steatotic media, proliferation media, or any other media formulation. Culture medium may be refreshed about every 1 hour (h), 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, or more than 24 hours, or any fraction thereof. In additional examples, the medium may be refreshed less often such as, but not limited to, every 1.1 days (d), 1.2 d, 1.3 d, 1.4 d, 1.5 d, 1.6 d, 1.7 d, 1.8 d, 1.9 d or every 2 or more days, or any time frame in between. As illustrated in FIG. 2, cells may be conditioned in a bioreactor, the bioreactor may be flushed with growth media, the cells seeded into a scaffold in the bioreactor with growth media to proliferate, differentiate media or mechanical stimulus may be added to differentiate the stem, precursor, or progenitor cells to mammary cells, a lactation media may be flushed in, and milk components may be extracted from the bioreactor.

Microfiltration/ultrafiltration, or any other separation technology can be used to remove media components from the liquid composition or milk obtained from the culture of the mammary cells, or mammary-like cells. Filtering may comprise using any type of filter that separate media components from the liquid composition or milk obtained from the culture of the mammary cells, or mammary-like cells such as carbon filtering or zeolite filtering. Filtration may comprise a hollow fiber, a tubular membrane. The internal diameter of a tubular membrane may be least about 0.1 millimeters (mm), 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, or more than 10 mm. In some cases, the internal diameter of a tubular membrane may be less than about 0.1 mm. Filter modules comprising hollow fibers may be commercially available from various commercial sources, including G.E. Life Sciences (Marlborough, Mass.) and InnovaPrep (Drexel, Mo.). Specific examples of hollow fiber filter systems that can be used, modified or adapted for use in the disclosed methods and systems include, but are not limited to, those described in U.S. Pat. Nos. 9,738,918; 9,593,359; 9,574,977; 9,534,989; 9,446,354; 9,295,824; 8,956,880; 8,758,623; 8,726,744; 8,677,839; 8,677,840; 8,584,536; 8,584,535; and 8,110,112, each of which is incorporated herein by reference in their entireties for all purposes. Filtered media may be recycled. Media after filtering may be replenished of lost nutrients and other media components before re-entering the bioreactor. Media recycling may comprise a closed-loop perfusion system, such as a dialysis unit permitting physiological addition of nutrients and removal of toxins. Media may be recycled at a predetermined time interval or based on an established benchmark such as cell density or composition of the conditioned medium. There may be a waste medium vessel or a fresh medium vessel in fluid communion with the bioreactor chambers. A waste medium vessel may collect media that is not recycled to facilitate draining and replacement of media in a controlled manner. A waste medium vessel may be in fluid communion to a dialyzer to filter waste medium and return the treated medium to the system. During media recycling a percentage of the medium may be removed and replaced with fresh basal medium added and/or used media removed, purified, and returned to a bioreactor chamber or fresh medium vessel.

A cultured dairy product may comprise or may be composed from milk. Milk may be a nutrient-rich liquid composition or food composition (or food product) produced in the mammary glands of mammals. Milk may be the primary source of nutrition for infant mammals. Milk may comprise a composition of growth factors, sugars, proteins, and fats. Milk may be consumed either as a liquid composition or a derivative of the liquid composition past infancy as a food composition or food product. Milk may comprise a variety of dairy products such as cream, butter, yogurt, kefir, ice cream, casein, lactose, whey protein, powdered milk, condensed milk, or cheese. Aspects of the present disclosure may comprise liquid compositions and methods of obtaining the same are disclosed herein whereby the liquid compositions may be obtained from the in vitro culture of mammary epithelial cells, or mammary-like cells, under conditions to promote the expression of at least one of a fat molecule, at least one of a whey protein and/or a casein protein, and at least one oligosaccharide. The expressed fat, protein and oligosaccharide molecules can be combined with water to form a nutrient composition (such as a liquid nutrient composition) having a liquid-fat emulsion component. As used herein, the term “liquid nutrient composition” may generally include any liquid product obtained from an in vitro culture of a mammary or mammary-like cell (e.g., a mammary epithelial cell) such as any described herein. As used herein, the terms “liquid composition,” “liquid nutrient composition,” “aqueous liquid composition,” “aqueous liquid nutrient composition,” and the like may be used interchangeably herein.

A liquid composition obtained by the culture of mammary epithelial cells or mammary-like cells may mimic the milk product obtained from a mammary organ; however, unlike the natural milk product, a liquid composition may be substantially free of an immunoglobulin, substantially free of whole cells, or substantially free of bacterial microbes. A liquid composition may mimic colostrum, early milk, transition milk, foremilk, hindmilk, etc. in terms of fat content, protein content, water content, or oligosaccharide content, and may be substantially free of an immunoglobulin, substantially free of whole cells, or substantially free of bacterial microbes. For example, and without limitation, aspects of the present liquid compositions can mimic mammalian colostrum, early milk, transition milk, mature milk, and the like in composition, taste, and nutritional benefit. As the liquid compositions obtained by the in vitro culture of mammary cells, or mammary-like cells, may be substantially free of an immunoglobulin, substantially free of whole cells, or substantially free of bacterial microbes, such resulting liquid compositions can be supplemented with additional components, molecules, or ingredients such as an immunoglobulin, prebiotic oligosaccharides, probiotic microbes, vitamin(s), immune enhancing cells such as leukocytes, or other components. The liquid compositions can be adjusted in ingredient or component concentration based on the desired end use of the liquid composition. Similarly, the liquid compositions can be made to substantially mimic early milk or transition milk from a mammal. Transition milk may be a combination of colostrum and mature milk. The liquid compositions described herein can have a protein content substantially the same or different as compared to natural colostrum, or a fat content substantially the same or different as compared to natural colostrum produced by the mammal. Such transition milk can be supplemented with additional components, molecules, or ingredients including, but not limited to, at least one immunoglobulin, such as Immunoglobulin A; probiotic microbes, such Pseudomonas, Staphylococcus, Streptococcus, Variovorax, Bifidobacterium, Flavobacterium, Lactobacillus, Stenotrophomonas, Brevundimonas, Chryseobacterium, and Enterobacter; immune enhancing cells such as leukocytes; or at least one vitamin.

### A Milk Nutrient Composition

A milk nutrient may comprise a milk protein (a protein) such as casein, whey protein, alpha-lactalbumin, lactoferrin, immunoglobulin IgA, lysozyme, or serum albumin. A milk nutrient may comprise a milk carbohydrate (a carbohydrate) such as lactose, monosaccharides, disaccharides, or oligosaccharides. A milk nutrient may comprise a milk fat (a fat) such as triacylglycerol, phospholipid, cholesterol, free fatty acid, monoglycerol, or diaglycerol. A milk nutrient may comprise a milk lipid (a lipid) such as long-chain polyunsaturated fatty acids such as docosahexaenoic (DHA) and arachidonic (ARA) acids. A milk nutrient may comprise an amino acid, a mineral, a vitamin, one or more bacteria (one or more probiotic bacteria), or an antibody.

A liquid composition may comprise a fat component. A fat component may be produced by mammary or mammary-like cells which may form a milk fat globule (MFG). A liquid composition may comprise an aqueous medium comprising one or more particles. A particle may be a lipid globule. There may be one or a plurality of lipid globules in a liquid composition. A particle of the one or more particles may comprise a fat surrounded by a layer. The MFG is generally composed of a triglyceride-rich core surrounded by a tri-layer membrane, also known as the milk fat globule membrane (MFGM) that originates from mammary epithelia. The layer may comprise a MFGM protein. The MFGM is enriched with glycerophospholipids, sphingolipids, cholesterol, and proteins, some of which may be glycosylated. MFGs may be heterogeneous structures, varying in diameter, triglyceride content, and membrane and fatty acid composition. The diameter of the MFG may be at least about 0.2 micrometers (m), 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, or greater than 15 μm and its composition can vary by size. The diameter of the MFG may be less than about 0.2 jun. A fat component may also be supplemented into the cultured dairy product. The one or more particles may be emulsified by the layer and dispersed in the aqueous medium.

A nutrient composition (such as a liquid nutrient composition) may comprise a protein. A protein may comprise casein protein. The casein protein content can contain four types of casein proteins αs1-casein, αs2-casein, β-casein, and κ-casein. A protein may comprise whey protein. The serum (whey) protein content can consist of at least one of a β-lactoglobulin, α-lactalbumin, blood serum albumin, immunoglobulins, lactoferrin, transferrin, minor proteins, and enzymes. For human mammary cells, or human mammary-like cells, the casein/whey protein produced is in the ratio of about 60% casein proteins to about 40% whey proteins. For dairy cow mammary cells, or dairy cow mammary-like cells, the casein/whey protein produced is in the ratio of about 80% casein proteins to about 20% whey proteins.

A nutrient composition (such as a liquid nutrient composition) may comprise at least one saccharide such as monosaccharides, disaccharides, or oligosaccharides. The monosaccharides that can be used as building blocks for oligosaccharides are glucose (Glc), galactose (Gal), N-Acetyl-Glucosamine (GlcNAc), fucose (Fuc), and sialic acid (Neu5Ac). A disaccharide may comprise maltose, sucrose and lactose. Lactose may be a disaccharide produced by mammary cells. The single monosaccharides may be conjugated via several linkage types (e.g., glycosidic bonds). With human mammary cells, each of the human milk oligosaccharides (HMOs) structure starts with a lactose unit “Gal β1-4) Glc” which results from formation of a β1-4 glycosidic linkage between galactose and glucose catalyzed by the lactose synthase protein complex. Several tri-saccharides can be synthesized by appending either galactose or fucose to the reducing or non-reducing end of the lactose residue, which is performed through galactosyl- or fucosyl-transferase activity. Resulting components may be for example lacto-N-neoetraose (LNnT), 3′-galactosyllactose (Gal β1-3)Gal(β1-4)Glc), 4′-galactosyllactose (Gal(β1-4)Gal(β1-4)Glc), 6′-galactosyllactose (Gal(β1-6)Gal(β1-4)Glc), 2′-FL (Fuc(α 1-2)Gal(β1-4)Glc), and 3-fucosyllactose (3′-FL) (Gal(β1-4)[Fucα1-3]Glc). If sialic acids are connected to the non-reducing end of lactose via sialyl-transferases, 3′-sialyllactose (3′-SL; Neu5Ac (α 2-3) Gal(β1-4) Glc) and 6′-sialyllactose (6′-SL) (Neu5Ac (α 2-6) Gal(β1-4) Glc) are formed. Further elongation of lactose via the free 3-OH group of galactose can occur by addition of Gal(β1-x) GlcNAc units of either type I (Gal (β1-3) GlcNAc, Lacto-N-biose) or type II (Gal ((31-4) GlcNAc, N-Acetyllactosamine). These core structures may be linear or branched and can be further decorated with fucoses or sialic acid residues. A liquid composition may comprise at least one of the above saccharides.

### Liquid Compositions

Aspects of the present disclosure may comprise a liquid composition obtained from an in vitro culture of mammary epithelial cells, the liquid composition comprising, by weight: about 25% to about 90% water, about 0.1% to about 20% of at least one protein, at most about 60% of at least one fat, at most about 30% of at least one carbohydrate, and about 0.0005% to about 3% of at least one mineral, wherein the liquid composition is supplemented with a nutritionally beneficial amount of a nutrient. The liquid composition may be substantially free of at least one of whole cells, at least one of an antibody, and at least one of a bacterial microbe. The liquid composition may comprise by weight about 0.1% to about 30% lactose. A mammary epithelial cell may be of human origin, bovine origin, dairy cattle cells, goat origin, buffalo origin, sheep origin, whale origin, seal origin, elephant origin, snow leopard origin, or any other mammalian species.

Aspects of the present disclosure may comprise a liquid composition comprising, by weight, about 25% to about 90% water, about 0.1% to about 20% of at least one protein, at most about 60% of at least one fat, at most about 30% of at least one carbohydrate, and about 0.0005% to about 3% of at least one mineral, and a nutritionally beneficial amount of at least one vitamin, wherein the liquid composition may be substantially free of antibodies, substantially free of bacterial microbes, and substantially free of whole cells.

A liquid composition may include, by weight, at least about 25% to about 30% water, about 25% water to about 30% water, about 25% water to about 40% water, about 25% to about 50% water, about 25% to about 60% water, about 25% to about 70% water, about 25% to about 80% water, about 25% to about 90% water, about 25% to more than 90% water, about 30% water to about 40% water, about 30% water to about 50% water, about 30% to about 60% water, about 30% to about 70% water, about 30% to about 80% water, about 30% to about 90% water, about 30% to more than 90% water, about 40% to about 50% water, about 40% to about 60% water, about 40% to about 70% water, about 40% to about 80% water, about 40% to about 90% water, about 40% to more than 90% water, about 50% to about 60% water, about 50% to about 70% water, about 50% to about 80% water, about 50% to about 90% water, about 50% to more than 90% water, about 60% to about 70% water, about 60% to about 80% water, about 60% to about 90% water, about 60% to about more than 90% water, about 70% to about 80% water, about 70% to about 90% water, about 70% to about more than 90% water, about 80% to about 90% water, about 80% to about more than 90% water, or about 90% water. A liquid composition may include, by weight, less than about 25% water to more than about 90% water.

A liquid composition may comprise, by weight, at least (about) 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% 15%, or 20% of at least one protein. A liquid composition may comprise, by weight, at most (about) 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% 15%, or 20% of at least one protein. A liquid composition may comprise, by weight, (about) 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% 15%, or 20%, or a range between any two of the foregoing values, of at least one protein. A liquid composition may comprise, by weight, less than (about) 0.1% of at least one protein. A liquid composition may comprise, by weight, at least (about) 1%, 2%, 3%, 4%, 5%, 6%, 7%, 8%, 9%, 10%, 15%, 20%, 25%, 30%, 35%, 40%, 45%, 50%, 55%, or 60% of at least one fat. A liquid composition may comprise, by weight, at most (about) 1%, 2%, 3%, 4%, 5%, 6%, 7%, 8%, 9%, 10%, 15%, 20%, 25%, 30%, 35%, 40%, 45%, 50%, 55%, or 60% of at least one fat. A liquid composition may comprise, by weight, (about) 1%, 2%, 3%, 4%, 5%, 6%, 7%, 8%, 9%, 10%, 15%, 20%, 25%, 30%, 35%, 40%, 45%, 50%, 55%, or 60%, or a range between any two of the foregoing values, of at least one fat. A liquid composition may comprise, by weight, at least (about) 1%, 2%, 3%, 4%, 5%, 6%, 7%, 8%, 9%, 10%, 15%, 20%, 25%, or 30% of at least one carbohydrate. A liquid composition may comprise, by weight, at most (about) 1%, 2%, 3%, 4%, 5%, 6%, 7%, 8%, 9%, 10%, 15%, 20%, 25%, or 30% of at least one carbohydrate. A liquid composition may comprise, by weight, (about) 1%, 2%, 3%, 4%, 5%, 6%, 7%, 8%, 9%, 10%, 15%, 20%, 25%, or 30%, or a range between any two of the foregoing values, of at least one carbohydrate. A liquid composition may comprise, by weight, at least (about) 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, or 3% of at least one mineral. A liquid composition may comprise, by weight, at most (about) 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, or 3% of at least one mineral. A liquid composition may comprise, by weight, (about) 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, or 3%, or a range between any two of the foregoing values, of at least one mineral. A liquid composition may comprise, by weight, less than about 0.0005% of at least one mineral. A liquid composition may comprise a nutritionally beneficial amount of at least one of Immunoglobulin IgA, lactoferrin, and a probiotic microbe.

The nutrient composition (such as a liquid nutrient composition) may comprise, by weight, about 40% to about 90% water, about 0.1% to about 15% of at least one protein, at most about 30% of at least one fat, about 0.1% to about 30% of at least one carbohydrate, and about 0.1% to about 3% of at least one mineral. A nutrient composition (such as a liquid nutrient composition) obtained from cultured cells may be substantially free of an immunoglobulin, substantially free of whole cells, or substantially free of bacterial microbes. A nutrient composition (such as a liquid nutrient composition) obtained from cultured cells may include, by weight, at least about 25% to about 30% water, about 25% water to about 30% water, about 25% water to about 40% water, about 25% to about 50% water, about 25% to about 60% water, about 25% to about 70% water, about 25% to about 80% water, about 25% to about 90% water, about 25% to more than 90% water, about 30% water to about 40% water, about 30% water to about 50% water, about 30% to about 60% water, about 30% to about 70% water, about 30% to about 80% water, about 30% to about 90% water, about 30% to more than 90% water, about 40% to about 50% water, about 40% to about 60% water, about 40% to about 70% water, about 40% to about 80% water, about 40% to about 90% water, about 40% to more than 90% water, about 50% to about 60% water, about 50% to about 70% water, about 50% to about 80% water, about 50% to about 90% water, about 50% to more than 90% water, about 60% to about 70% water, about 60% to about 80% water, about 60% to about 90% water, about 60% to about more than 90% water, about 70% to about 80% water, about 70% to about 90% water, about 70% to about more than 90% water, about 80% to about 90% water, about 80% to about more than 90% water, or more than 90% water. A nutrient composition may include, by weight, less than about 25% water.

A liquid composition may comprise, by weight, about 40% to about 90% water, about 3% to about 7% of at least one protein, about 3% to about 8% of at least one fat, about 1% to about 5% of at least one carbohydrate, and about 0.1% to about 1% of at least one mineral. A liquid composition may comprise, by weight, about 40% to about 90% water, about 1% to about 2% of at least one protein, about 3% to about 5% of at least one fat, about 7% to about 8% of at least one carbohydrate, and about 0.1% to about 1% of at least one mineral.

A nutrient composition (such as a liquid nutrient composition) obtained from cultured cells may comprise, by weight, at least (about) 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% 15%, or 20% of at least one protein. A nutrient composition (such as a liquid nutrient composition) obtained from cultured cells may comprise, by weight, at most (about) 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% 15%, or 20% of at least one protein. A nutrient composition (such as a liquid nutrient composition) obtained from cultured cells may comprise, by weight, (about) 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% 15%, or 20%, or a range between any two of the foregoing values, of at least one protein. A nutrient composition (such as a liquid nutrient composition) obtained from cultured cells may comprise, by weight, less than about 0.1% of at least one protein. A nutrient composition (such as a liquid nutrient composition) obtained from cultured cells may comprise, by weight, at least about 1%, 2%, 3%, 4%, 5%, 6%, 7%, 8%, 9%, 10%, 15%, 20%, 25%, 30%, 35%, 40%, 45%, 50%, 55%, or 60% of at least one fat. A nutrient composition (such as a liquid nutrient composition) obtained from cultured cells may comprise, by weight, at most about 1%, 2%, 3%, 4%, 5%, 6%, 7%, 8%, 9%, 10%, 15%, 20%, 25%, 30%, 35%, 40%, 45%, 50%, 55%, or 60% of at least one fat. A nutrient composition (such as a liquid nutrient composition) obtained from cultured cells may comprise, by weight, about 1%, 2%, 3%, 4%, 5%, 6%, 7%, 8%, 9%, 10%, 15%, 20%, 25%, 30%, 35%, 40%, 45%, 50%, 55%, or 60%, or a range between any two of the foregoing values, of at least one fat. A nutrient composition (such as a liquid nutrient composition) obtained from cultured cells may comprise, by weight, at least about 1%, 2%, 3%, 4%, 5%, 6%, 7%, 8%, 9%, 10%, 15%, 20%, 25%, or 30% of at least one carbohydrate. A nutrient composition (such as a liquid nutrient composition) obtained from cultured cells may comprise, by weight, at most about 1%, 2%, 3%, 4%, 5%, 6%, 7%, 8%, 9%, 10%, 15%, 20%, 25%, or 30% of at least one carbohydrate. A nutrient composition (such as a liquid nutrient composition) obtained from cultured cells may comprise, by weight, about 1%, 2%, 3%, 4%, 5%, 6%, 7%, 8%, 9%, 10%, 15%, 20%, 25%, or 30%, or a range between any two of the foregoing values, of at least one carbohydrate. A nutrient composition (such as a liquid nutrient composition) obtained from cultured cells may comprise, by weight, at least about 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, or 3% of at least one mineral. A nutrient composition (such as a liquid nutrient composition) obtained from cultured cells may comprise, by weight, at most about 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, or 3% of at least one mineral. A nutrient composition (such as a liquid nutrient composition) obtained from cultured cells may comprise, by weight, about 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, or 3%, or a range between any two of the foregoing values, of at least one mineral. A nutrient composition (such as a liquid nutrient composition) obtained from cultured cells may comprise, by weight, less than about 0.0005% of at least one mineral. The at least one mineral may comprise calcium. A nutrient composition (such as a liquid nutrient composition) obtained from cultured cells may comprise, by weight, at least about 0.005%, 0.006%, 0.007%, 0.008%, 0.009%, 0.01%, 02%, 0.03%, 0.04%, 0.05%, 0.06%, 0.07%, 0.08%, 0.09%, 0.1%, 0.11%, or 0.12% calcium. A nutrient composition (such as a liquid nutrient composition) obtained from cultured cells may comprise, by weight, at most about 0.005%, 0.006%, 0.007%, 0.008%, 0.009%, 0.01%, 02%, 0.03%, 0.04%, 0.05%, 0.06%, 0.07%, 0.08%, 0.09%, 0.1%, 0.11%, or 0.12% calcium. A nutrient composition (such as a liquid nutrient composition) obtained from cultured cells may comprise, by weight, about 0.005%, 0.006%, 0.007%, 0.008%, 0.009%, 0.01%, 02%, 0.03%, 0.04%, 0.05%, 0.06%, 0.07%, 0.08%, 0.09%, 0.1%, 0.11%, or 0.12%, or a range of any two of the foregoing values, calcium. A nutrient composition (such as a liquid nutrient composition) obtained from cultured cells may comprise, by weight, less than 0.005% calcium. The at least one mineral may comprise magnesium. A nutrient composition (such as a liquid nutrient composition) obtained from cultured cells may comprise, by weight, at least about 0.001%, 0.002%, 0.003%, 0.004%, 0.005%, 0.006%, 0.007%, 0.008%, 0.009%, 0.01%, 0.02%, 0.03%, 0.04%, or 0.05% of magnesium. A nutrient composition (such as a liquid nutrient composition) obtained from cultured cells may comprise, by weight, at most about 0.001%, 0.002%, 0.003%, 0.004%, 0.005%, 0.006%, 0.007%, 0.008%, 0.009%, 0.01%, 0.02%, 0.03%, 0.04%, or 0.05% of magnesium. A nutrient composition (such as a liquid nutrient composition) obtained from cultured cells may comprise, by weight, about 0.001%, 0.002%, 0.003%, 0.004%, 0.005%, 0.006%, 0.007%, 0.008%, 0.009%, 0.01%, 0.02%, 0.03%, 0.04%, or 0.05%, or a range of any two of the foregoing values, of magnesium. A nutrient composition (such as a liquid nutrient composition) obtained from cultured cells may comprise, by weight, less than about 0.001% of magnesium. A nutrient composition (such as a liquid nutrient composition) obtained from cultured cells may comprise, by weight, at least about 0.000001%, 0.000002%, 0.000003%, 0.000004%, 0.000005%, 0.000006%, 0.000007%, 0.000008%, 0.000009%, 0.00001%, 0.00002%, 0.00003%, 0.00004%, or 0.00005% of selenium. A nutrient composition (such as a liquid nutrient composition) obtained from cultured cells may comprise, by weight, at most about 0.000001%, 0.000002%, 0.000003%, 0.000004%, 0.000005%, 0.000006%, 0.000007%, 0.000008%, 0.000009%, 0.00001%, 0.00002%, 0.00003%, 0.00004%, or 0.00005% of selenium. A nutrient composition (such as a liquid nutrient composition) obtained from cultured cells may comprise, by weight, at least about 0.000001%, 0.000002%, 0.000003%, 0.000004%, 0.000005%, 0.000006%, 0.000007%, 0.000008%, 0.000009%, 0.00001%, 0.00002%, 0.00003%, 0.00004%, or 0.00005%, ora range of any two of the foregoing values, of selenium. A nutrient composition (such as a liquid nutrient composition) obtained from cultured cells may comprise, by weight, less than about 0.000001% of selenium. A nutrient composition (such as a liquid nutrient composition) obtained from cultured cells may comprise, by weight, at least about 0.0005%, 0.0006%, 0.0007%, 0.0008%, 0.0009%, 0.001%, 0.002%, 0.003%, 0.004%, 0.005%, 0.006%, 0.007%, 0.008%, 0.009%, or 0.01% of zinc. A nutrient composition (such as a liquid nutrient composition) obtained from cultured cells may comprise, by weight, at most about 0.0005%, 0.0006%, 0.0007%, 0.0008%, 0.0009%, 0.001%, 0.002%, 0.003%, 0.004%, 0.005%, 0.006%, 0.007%, 0.008%, 0.009%, or 0.01% of zinc. A nutrient composition (such as a liquid nutrient composition) obtained from cultured cells may comprise, by weight, at least about 0.0005%, 0.0006%, 0.0007%, 0.0008%, 0.0009%, 0.001%, 0.002%, 0.003%, 0.004%, 0.005%, 0.006%, 0.007%, 0.008%, 0.009%, or 0.01%, or a range of any two of the foregoing values, of zinc. A nutrient composition (such as a liquid nutrient composition) obtained from cultured cells may comprise, by weight, less than about 0.0005% of zinc. A nutrient composition (such as a liquid nutrient composition) may comprise an emulsion of at least one lipid and a fluid containing protein(s), sugar(s), lipid(s), mineral(s), or water.

Liquid compositions obtained from the in vitro culturing of bovine mammary epithelial cells, or bovine mammary-like cells, may be substantially free of an immunoglobulin, substantially free of whole cells, or substantially free of bacterial microbes. Such a liquid composition can include, by weight, at least about 40% to about 50% water, about 40% to about 50% water, about 40% to about 60% water, about 40% to about 70% water, about 40% to about 80% water, about 40% to about 90% water, about 40% to more than 90% water, about 50% to about 60% water, about 50% to about 70% water, about 50% to about 80% water, about 50% to about 90% water, about 50% to more than 90% water, about 60% to about 70% water, about 60% to about 80% water, about 60% to about 90% water, about 60% to about more than 90% water, about 70% to about 80% water, about 70% to about 90% water, about 70% to about more than 90% water, about 80% to about 90% water, about 80% to about more than 90% water, or about more than 90% water. A liquid composition can include, by weight, less than about 40% water.

Liquid compositions may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% lactose. Liquid compositions may comprise, by weight, at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% lactose. Liquid compositions may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10%, or a range between any two of the foregoing values, lactose. Liquid compositions may comprise, by weight, less than 0.1% lactose. Liquid compositions may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% protein. Liquid compositions may comprise, by weight, at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% protein. Liquid compositions may comprise, by weight, about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10%, or a range between any two of the foregoing values, protein. Liquid compositions may comprise, by weight, less than 0.1% protein. Liquid compositions may comprise, by weight, at least about 1%, 2%, 3%, 4%, 5%, 6%, 7%, 8%, 9%, 10%, 15%, 20%, 25%, 30%, 35%, 40%, 45%, 50%, 55%, or 60% fat. Liquid compositions may comprise, by weight, less than 0.1% protein. Liquid compositions may comprise, by weight, at most about 1%, 2%, 3%, 4%, 5%, 6%, 7%, 8%, 9%, 10%, 15%, 20%, 25%, 30%, 35%, 40%, 45%, 50%, 55%, or 60% fat. Liquid compositions may comprise, by weight, less than 0.1% protein. Liquid compositions may comprise, by weight, at least about 1%, 2%, 3%, 4%, 5%, 6%, 7%, 8%, 9%, 10%, 15%, 20%, 25%, 30%, 35%, 40%, 45%, 50%, 55%, or 60%, or a range between any two of the foregoing values, fat. Liquid compositions may comprise, by weight, at least about 0.1%, 0.2%, 0.3%, 0.4%, 0.5%, 0.6%, 0.7%, 0.8%, 0.9%, 1%, 2%, or 3% mineral(s). Liquid compositions may comprise, by weight, at most about 0.1%, 0.2%, 0.3%, 0.4%, 0.5%, 0.6%, 0.7%, 0.8%, 0.9%, 1%, 2%, or 3% mineral(s). Liquid compositions may comprise, by weight, at least about 0.1%, 0.2%, 0.3%, 0.4%, 0.5%, 0.6%, 0.7%, 0.8%, 0.9%, 1%, 2%, or 3%, or a range between any two of the foregoing values, mineral(s). Liquid compositions may comprise, by weight, less than 0.1% mineral(s).

Liquid compositions obtained from the in vitro culturing of goat mammary epithelial cells, or goat mammary-like cells, may be substantially free of an immunoglobulin, substantially free of whole cells, or substantially free of bacterial microbes. Liquid compositions obtained from the in vitro culturing of goat mammary epithelial cells, or goat mammary-like cells can include, by weight, at least about 40% to about 50% water, about 40% to about 50% water, about 40% to about 60% water, about 40% to about 70% water, about 40% to about 80% water, about 40% to about 90% water, about 40% to more than 90% water, about 50% to about 60% water, about 50% to about 70% water, about 50% to about 80% water, about 50% to about 90% water, about 50% to more than 90% water, about 60% to about 70% water, about 60% to about 80% water, about 60% to about 90% water, about 60% to about more than 90% water, about 70% to about 80% water, about 70% to about 90% water, about 70% to about more than 90% water, about 80% to about 90% water, about 80% to about more than 90% water, or about more than 90% water. Liquid compositions obtained from the in vitro culturing of goat mammary epithelial cells, or goat mammary-like cells can include, by weight, less than about 40% water.

Liquid compositions obtained from the in vitro culturing of goat mammary epithelial cells, or goat mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, or more than 20% lactose. Liquid compositions obtained from the in vitro culturing of goat mammary epithelial cells, or goat mammary-like cells, may comprise, by weight, at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, or 20% lactose. Liquid compositions obtained from the in vitro culturing of goat mammary epithelial cells, or goat mammary-like cells, may comprise, by weight, about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, or 20%, or a range between any two of the foregoing values, lactose. Liquid compositions obtained from the in vitro culturing of goat mammary epithelial cells, or goat mammary-like cells, may comprise, by weight, less than about 0.1% lactose. Liquid compositions obtained from the in vitro culturing of goat mammary epithelial cells, or goat mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, or more than 15% protein. Liquid compositions obtained from the in vitro culturing of goat mammary epithelial cells, or goat mammary-like cells, may comprise, by weight, at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or 15% protein. Liquid compositions obtained from the in vitro culturing of goat mammary epithelial cells, or goat mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or 15%, or a range between any two of the foregoing values, protein. Liquid compositions obtained from the in vitro culturing of goat mammary epithelial cells, or goat mammary-like cells, may comprise, by weight, less than about 0.1% protein. Liquid compositions obtained from the in vitro culturing of goat mammary epithelial cells, or goat mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11% 12% or more than 12% fat. Liquid compositions obtained from the in vitro culturing of goat mammary epithelial cells, or goat mammary-like cells, may comprise, by weight, at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, or 12% fat. Liquid compositions obtained from the in vitro culturing of goat mammary epithelial cells, or goat mammary-like cells, may comprise, by weight, at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, or 12%, or a range between any two of the foregoing values, fat. Liquid compositions obtained from the in vitro culturing of goat mammary epithelial cells, or goat mammary-like cells, may comprise, by weight, less than about 0.1% fat. Liquid compositions obtained from the in vitro culturing of goat mammary epithelial cells, or goat mammary-like cells, may comprise, by weight, about comprise at least about 0.1%, 0.2%, 0.3%, 0.4%, 0.5%, 0.6%, 0.7%, 0.8%, 0.9%, 1%, 2%, 3%, or more than 3% mineral(s). Liquid compositions obtained from the in vitro culturing of goat mammary epithelial cells, or goat mammary-like cells, may comprise, by weight, about comprise at most about 0.1%, 0.2%, 0.3%, 0.4%, 0.5%, 0.6%, 0.7%, 0.8%, 0.9%, 1%, 2%, or 3% mineral(s). Liquid compositions obtained from the in vitro culturing of goat mammary epithelial cells, or goat mammary-like cells, may comprise, by weight, about comprise at most about 0.1%, 0.2%, 0.3%, 0.4%, 0.5%, 0.6%, 0.7%, 0.8%, 0.9%, 1%, 2%, or 3%, or a range between any two of the foregoing values, mineral(s). Liquid compositions obtained from the in vitro culturing of goat mammary epithelial cells, or goat mammary-like cells, may comprise, by weight, less than about 0.1% mineral(s).

Liquid compositions obtained from the in vitro culturing of human mammary epithelial cells, or human mammary-like cells, may be substantially free of an immunoglobulin, substantially free of whole cells, or substantially free of bacterial microbes. Liquid compositions obtained from the in vitro culturing of human mammary epithelial cells, or human mammary-like cells can include, by weight, at least about 40% to about 50% water, about 40% to about 50% water, about 40% to about 60% water, about 40% to about 70% water, about 40% to about 80% water, about 40% to about 90% water, about 40% to more than 90% water, about 50% to about 60% water, about 50% to about 70% water, about 50% to about 80% water, about 50% to about 90% water, about 50% to more than 90% water, about 60% to about 70% water, about 60% to about 80% water, about 60% to about 90% water, about 60% to about more than 90% water, about 70% to about 80% water, about 70% to about 90% water, about 70% to about more than 90% water, about 80% to about 90% water, or about more than 90% water. Liquid compositions obtained from the in vitro culturing of human mammary epithelial cells, or human mammary-like cells can include, by weight, less than 40% water.

Liquid compositions obtained from the in vitro culturing of human mammary epithelial cells, or human mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, or 30% lactose. Liquid compositions obtained from the in vitro culturing of human mammary epithelial cells, or human mammary-like cells, may comprise, by weight, at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, or 30% lactose. Liquid compositions obtained from the in vitro culturing of human mammary epithelial cells, or human mammary-like cells, may comprise, by weight, about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, or 30% lactose. Liquid compositions obtained from the in vitro culturing of human mammary epithelial cells, or human mammary-like cells, may comprise, by weight, less than about 0.1% lactose. Liquid compositions obtained from the in vitro culturing of human mammary epithelial cells, or human mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, or 8% protein. Liquid compositions obtained from the in vitro culturing of human mammary epithelial cells, or human mammary-like cells, may comprise, by weight, at most about 0.1%, 0.2%, 0.3%, 0.4%, 0.5%, 0.6%, 0.7%, 0.8%, 0.9%, 1%, 2%, 3%, 4%, 5%, 6%, 7%, or 8% protein. Liquid compositions obtained from the in vitro culturing of human mammary epithelial cells, or human mammary-like cells, may comprise, by weight, about 0.1%, 0.2%, 0.3%, 0.4%, 0.5%, 0.6%, 0.7%, 0.8%, 0.9%, 1%, 2%, 3%, 4%, 5%, 6%, 7%, or 8% protein. Liquid compositions obtained from the in vitro culturing of human mammary epithelial cells, or human mammary-like cells, may comprise, by weight, less than about 0.1% protein. Liquid compositions obtained from the in vitro culturing of human mammary epithelial cells, or human mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% fat. Liquid compositions obtained from the in vitro culturing of human mammary epithelial cells, or human mammary-like cells, may comprise, by weight, at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% fat. Liquid compositions obtained from the in vitro culturing of human mammary epithelial cells, or human mammary-like cells, may comprise, by weight, about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% fat. Liquid compositions obtained from the in vitro culturing of human mammary epithelial cells, or human mammary-like cells, may comprise, by weight, less than about 0.1% fat. Liquid compositions obtained from the in vitro culturing of human mammary epithelial cells, or human mammary-like cells, may comprise, by weight, at least about 0.1%, 0.2%, 0.3%, 0.4%, 0.5%, 0.6%, 0.7%, 0.8%, 0.9%, 1%, 2%, or 3% mineral(s). Liquid compositions obtained from the in vitro culturing of human mammary epithelial cells, or human mammary-like cells, may comprise, by weight, at most about 0.1%, 0.2%, 0.3%, 0.4%, 0.5%, 0.6%, 0.7%, 0.8%, 0.9%, 1%, 2%, or 3% mineral(s). Liquid compositions obtained from the in vitro culturing of human mammary epithelial cells, or human mammary-like cells, may comprise, by weight, about 0.1%, 0.2%, 0.3%, 0.4%, 0.5%, 0.6%, 0.7%, 0.8%, 0.9%, 1%, 2%, or 3% mineral(s). Liquid compositions obtained from the in vitro culturing of human mammary epithelial cells, or human mammary-like cells, may comprise, by weight, less than about 0.1% mineral(s).

Liquid compositions obtained from the in vitro culturing of sheep mammary epithelial cells, or sheep mammary-like cells, may be substantially free of an immunoglobulin, substantially free of whole cells, or substantially free of bacterial microbes. Liquid compositions obtained from the in vitro culturing of sheep mammary epithelial cells, or sheep mammary-like cells can include, by weight, at least about 40% to about 50% water, about 40% to about 50% water, about 40% to about 60% water, about 40% to about 70% water, about 40% to about 80% water, about 40% to about 90% water, about 40% to more than 90% water, about 50% to about 60% water, about 50% to about 70% water, about 50% to about 80% water, about 50% to about 90% water, about 50% to more than 90% water, about 60% to about 70% water, about 60% to about 80% water, about 60% to about 90% water, about 60% to about more than 90% water, about 70% to about 80% water, about 70% to about 90% water, about 70% to about more than 90% water, about 80% to about 90% water, about 80% to about more than 90% water, or about more than 90% water. Liquid compositions obtained from the in vitro culturing of sheep mammary epithelial cells, or sheep mammary-like cells can include, by weight, less than 40% water.

Liquid compositions obtained from the in vitro culturing of sheep mammary epithelial cells, or sheep mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, or 25% lactose. Liquid compositions obtained from the in vitro culturing of sheep mammary epithelial cells, or sheep mammary-like cells, may comprise, by weight, less than about 0.1% lactose. Liquid compositions obtained from the in vitro culturing of sheep mammary epithelial cells, or sheep mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, or 6% protein. Liquid compositions obtained from the in vitro culturing of sheep mammary epithelial cells, or sheep mammary-like cells, may comprise, by weight, less than about 0.1% protein. Liquid compositions obtained from the in vitro culturing of sheep mammary epithelial cells, or sheep mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15% 20%, or 25% fat. Liquid compositions obtained from the in vitro culturing of sheep mammary epithelial cells, or sheep mammary-like cells, may comprise, by weight, less than about 0.1% fat. Liquid compositions obtained from the in vitro culturing of sheep mammary epithelial cells, or sheep mammary-like cells, may comprise, by weight, about comprise at least about 0.1%, 0.2%, 0.3%, 0.4%, 0.5%, 0.6%, 0.7%, 0.8%, 0.9%, 1%, 2%, or 3% mineral(s). Liquid compositions obtained from the in vitro culturing of sheep mammary epithelial cells, or sheep mammary-like cells, may comprise, by weight, less than about 0.1% mineral(s).

Liquid compositions obtained from the in vitro culturing of buffalo mammary epithelial cells, or buffalo mammary-like cells, may be substantially free of an immunoglobulin, substantially free of whole cells, or substantially free of bacterial microbes. Liquid compositions obtained from the in vitro culturing of buffalo mammary epithelial cells, or buffalo mammary-like cells can include, by weight, at least about 40% to about 50% water, about 40% to about 50% water, about 40% to about 60% water, about 40% to about 70% water, about 40% to about 80% water, about 40% to about 90% water, about 40% to more than 90% water, about 50% to about 60% water, about 50% to about 70% water, about 50% to about 80% water, about 50% to about 90% water, about 50% to more than 90% water, about 60% to about 70% water, about 60% to about 80% water, about 60% to about 90% water, about 60% to about more than 90% water, about 70% to about 80% water, about 70% to about 90% water, about 70% to about more than 90% water, about 80% to about 90% water, about 80% to about more than 90% water, or about more than 90% water. Liquid compositions obtained from the in vitro culturing of buffalo mammary epithelial cells, or buffalo mammary-like cells can include, by weight, less than about 40% water.

Liquid compositions obtained from the in vitro culturing of buffalo mammary epithelial cells, or buffalo mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or 15% lactose. Liquid compositions obtained from the in vitro culturing of buffalo mammary epithelial cells, or buffalo mammary-like cells, may comprise, by weight, less than about 0.1% lactose. Liquid compositions obtained from the in vitro culturing of buffalo mammary epithelial cells, or buffalo mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% 10%, 15%, or 20% protein. Liquid compositions obtained from the in vitro culturing of buffalo mammary epithelial cells, or buffalo mammary-like cells, may comprise, by weight, less than about 0.1% protein. Liquid compositions obtained from the in vitro culturing of buffalo mammary epithelial cells, or buffalo mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, or 25% fat. Liquid compositions obtained from the in vitro culturing of buffalo mammary epithelial cells, or buffalo mammary-like cells, may comprise, by weight, less than about 0.1% fat. Liquid compositions obtained from the in vitro culturing of buffalo mammary epithelial cells, or buffalo mammary-like cells, may comprise, by weight, about comprise at least about 0.1%, 0.2%, 0.3%, 0.4%, 0.5%, 0.6%, 0.7%, 0.8%, 0.9%, 1%, 2%, or 3% mineral(s). Liquid compositions obtained from the in vitro culturing of buffalo mammary epithelial cells, or buffalo mammary-like cells, may comprise, by weight, less than about 0.1% mineral(s).

Liquid compositions obtained from the in vitro culturing of camel mammary epithelial cells, or camel mammary-like cells, may be substantially free of an immunoglobulin, substantially free of whole cells, or substantially free of bacterial microbes. Liquid compositions obtained from the in vitro culturing of camel mammary epithelial cells, or camel mammary-like cells can include, by weight, at least about 40% to about 50% water, about 40% to about 50% water, about 40% to about 60% water, about 40% to about 70% water, about 40% to about 80% water, about 40% to about 90% water, about 40% to more than 90% water, about 50% to about 60% water, about 50% to about 70% water, about 50% to about 80% water, about 50% to about 90% water, about 50% to more than 90% water, about 60% to about 70% water, about 60% to about 80% water, about 60% to about 90% water, about 60% to about more than 90% water, about 70% to about 80% water, about 70% to about 90% water, about 70% to about more than 90% water, about 80% to about 90% water, about 80% to about more than 90% water, or about more than 90% water. Liquid compositions obtained from the in vitro culturing of camel mammary epithelial cells, or camel mammary-like cells can include, by weight, less than about 40% water.

Liquid compositions obtained from the in vitro culturing of camel mammary epithelial cells, or camel mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, or 12% lactose. Liquid compositions obtained from the in vitro culturing of camel mammary epithelial cells, or camel mammary-like cells, may comprise, by weight, less than about 0.1% lactose. Liquid compositions obtained from the in vitro culturing of camel mammary epithelial cells, or camel mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% protein. Liquid compositions obtained from the in vitro culturing of camel mammary epithelial cells, or camel mammary-like cells, may comprise, by weight, less than about 0.1% protein. Liquid compositions obtained from the in vitro culturing of camel mammary epithelial cells, or camel mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or 15% fat. Liquid compositions obtained from the in vitro culturing of camel mammary epithelial cells, or camel mammary-like cells, may comprise, by weight, less than about 0.1% fat. Liquid compositions obtained from the in vitro culturing of camel mammary epithelial cells, or camel mammary-like cells, may comprise, by weight, about comprise at least about 0.1%, 0.2%, 0.3%, 0.4%, 0.5%, 0.6%, 0.7%, 0.8%, 0.9%, 1%, 2%, or 3% mineral(s). Liquid compositions obtained from the in vitro culturing of camel mammary epithelial cells, or camel mammary-like cells, may comprise, by weight, less than about 0.1% mineral(s).

Liquid compositions obtained from the in vitro culturing of donkey mammary epithelial cells, or donkey mammary-like cells, may be substantially free of an immunoglobulin, substantially free of whole cells, or substantially free of bacterial microbes. Liquid compositions obtained from the in vitro culturing of donkey mammary epithelial cells, or donkey mammary-like cells can include, by weight, at least about 40% to about 50% water, about 40% to about 50% water, about 40% to about 60% water, about 40% to about 70% water, about 40% to about 80% water, about 40% to about 90% water, about 40% to more than 90% water, about 50% to about 60% water, about 50% to about 70% water, about 50% to about 80% water, about 50% to about 90% water, about 50% to more than 90% water, about 60% to about 70% water, about 60% to about 80% water, about 60% to about 90% water, about 60% to about more than 90% water, about 70% to about 80% water, about 70% to about 90% water, about 70% to about more than 90% water, about 80% to about 90% water, about 80% to about more than 90% water, or about more than 90% water. Liquid compositions obtained from the in vitro culturing of donkey mammary epithelial cells, or donkey mammary-like cells can include, by weight, less than about 40% water.

Liquid compositions obtained from the in vitro culturing of donkey mammary epithelial cells, or donkey mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, or 20% lactose. Liquid compositions obtained from the in vitro culturing of donkey mammary epithelial cells, or donkey mammary-like cells, may comprise, by weight, less than about 0.1% lactose. Liquid compositions obtained from the in vitro culturing of donkey mammary epithelial cells, or donkey mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, or 9% protein. Liquid compositions obtained from the in vitro culturing of donkey mammary epithelial cells, or donkey mammary-like cells, may comprise, by weight, less than about 0.1% protein. Liquid compositions obtained from the in vitro culturing of donkey mammary epithelial cells, or donkey mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, or 9% fat. Liquid compositions obtained from the in vitro culturing of donkey mammary epithelial cells, or donkey mammary-like cells, may comprise, by weight, less than about 0.1% fat. Liquid compositions obtained from the in vitro culturing of donkey mammary epithelial cells, or donkey mammary-like cells, may comprise, by weight, about comprise at least about 0.1%, 0.2%, 0.3%, 0.4%, 0.5%, 0.6%, 0.7%, 0.8%, 0.9%, 1%, 2%, or 3% mineral(s). Liquid compositions obtained from the in vitro culturing of donkey mammary epithelial cells, or donkey mammary-like cells, may comprise, by weight, less than about 0.1% mineral(s).

Liquid compositions obtained from the in vitro culturing of elephant mammary epithelial cells, or elephant mammary-like cells, may be substantially free of an immunoglobulin, substantially free of whole cells, or substantially free of bacterial microbes. Liquid compositions obtained from the in vitro culturing of elephant mammary epithelial cells, or elephant mammary-like cells can include, by weight, at least about 40% to about 50% water, about 40% to about 50% water, about 40% to about 60% water, about 40% to about 70% water, about 40% to about 80% water, about 40% to about 90% water, about 40% to more than 90% water, about 50% to about 60% water, about 50% to about 70% water, about 50% to about 80% water, about 50% to about 90% water, about 50% to more than 90% water, about 60% to about 70% water, about 60% to about 80% water, about 60% to about 90% water, about 60% to about more than 90% water, about 70% to about 80% water, about 70% to about 90% water, about 70% to about more than 90% water, about 80% to about 90% water, about 80% to about more than 90% water, or about more than 90% water. Liquid compositions obtained from the in vitro culturing of elephant mammary epithelial cells, or elephant mammary-like cells can include, by weight, less than about 40% water.

Liquid compositions obtained from the in vitro culturing of elephant mammary epithelial cells, or elephant mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 21%, or 22% lactose. Liquid compositions obtained from the in vitro culturing of elephant mammary epithelial cells, or elephant mammary-like cells, may comprise, by weight, less than about 0.1% lactose. Liquid compositions obtained from the in vitro culturing of elephant mammary epithelial cells, or elephant mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, or 16% protein. Liquid compositions obtained from the in vitro culturing of elephant mammary epithelial cells, or elephant mammary-like cells, may comprise, by weight, less than about 0.1% protein. Liquid compositions obtained from the in vitro culturing of elephant mammary epithelial cells, or elephant mammary-like cells, may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, or 25% fat. Liquid compositions obtained from the in vitro culturing of elephant mammary epithelial cells, or elephant mammary-like cells, may comprise, by weight, less than about 0.1% fat. Liquid compositions obtained from the in vitro culturing of elephant mammary epithelial cells, or elephant mammary-like cells, may comprise, by weight, about comprise at least about 0.1%, 0.2%, 0.3%, 0.4%, 0.5%, 0.6%, 0.7%, 0.8%, 0.9%, 1%, 2%, or 3% mineral(s). Liquid compositions obtained from the in vitro culturing of elephant mammary epithelial cells, or elephant mammary-like cells, may comprise, by weight, less than about 0.1% mineral(s).

### Liquid Compositions

Aspects of the present disclosure may comprise a food composition or food product comprising a composition obtained from an in vitro culture of mammary epithelial cells, the composition comprising, by weight, about 25% to about 90% water, about 0.1% to about 20% of at least one protein, at most about 60% of at least one fat, at most about 30% of at least one carbohydrate, and about 0.0005% to about 3% of at least one mineral.

A food composition or food product comprising a composition obtained from an in vitro culture of mammary epithelial cells may include, by weight, at least about 25% to about 30% water, about 25% water to about 30% water, about 25% water to about 40% water, about 25% to about 50% water, about 25% to about 60% water, about 25% to about 70% water, about 25% to about 80% water, about 25% to about 90% water, about 25% to more than 90% water, about 30% water to about 40% water, about 30% water to about 50% water, about 30% to about 60% water, about 30% to about 70% water, about 30% to about 80% water, about 30% to about 90% water, about 30% to more than 90% water, about 40% to about 50% water, about 40% to about 60% water, about 40% to about 70% water, about 40% to about 80% water, about 40% to about 90% water, about 40% to more than 90% water, about 50% to about 60% water, about 50% to about 70% water, about 50% to about 80% water, about 50% to about 90% water, about 50% to more than 90% water, about 60% to about 70% water, about 60% to about 80% water, about 60% to about 90% water, about 60% to about more than 90% water, about 70% to about 80% water, about 70% to about 90% water, about 70% to about more than 90% water, about 80% to about 90% water, about 80% to about more than 90% water, or about more than 90% water. A food composition or food product comprising a composition obtained from an in vitro culture of mammary epithelial cells may include, by weight, less than about 25% water.

A food composition or food product comprising a composition obtained from an in vitro culture of mammary epithelial cells may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% 15%, or 20% of at least one protein. A food composition or food product comprising a composition obtained from an in vitro culture of mammary epithelial cells may comprise, by weight, at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% 15%, or 20% of at least one protein. A food composition or food product comprising a composition obtained from an in vitro culture of mammary epithelial cells may comprise, by weight, about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% 15%, or 20%, or a range between any two of the foregoing values, of at least one protein. A food composition or food product comprising a composition obtained from an in vitro culture of mammary epithelial cells may comprise, by weight, less than about 0.1% of at least one protein. A food composition or food product comprising a composition obtained from an in vitro culture of mammary epithelial cells may comprise, by weight, at least about 1%, 2%, 3%, 4%, 5%, 6%, 7%, 8%, 9%, 10%, 15%, 20%, 25%, 30%, 35%, 40%, 45%, 50%, 55%, or 60% of at least one fat. A food composition or food product comprising a composition obtained from an in vitro culture of mammary epithelial cells may comprise, by weight, at most about 1%, 2%, 3%, 4%, 5%, 6%, 7%, 8%, 9%, 10%, 15%, 20%, 25%, 30%, 35%, 40%, 45%, 50%, 55%, or 60% of at least one fat. A food composition or food product comprising a composition obtained from an in vitro culture of mammary epithelial cells may comprise, by weight, about 1%, 2%, 3%, 4%, 5%, 6%, 7%, 8%, 9%, 10%, 15%, 20%, 25%, 30%, 35%, 40%, 45%, 50%, 55%, or 60%, or a range between any two of the foregoing values, of at least one fat. A food composition or food product comprising a composition obtained from an in vitro culture of mammary epithelial cells may comprise, by weight, at least about 1%, 2%, 3%, 4%, 5%, 6%, 7%, 8%, 9%, 10%, 15%, 20%, 25%, or 30% of at least one carbohydrate. A food composition or food product comprising a composition obtained from an in vitro culture of mammary epithelial cells may comprise, by weight, at most about 1%, 2%, 3%, 4%, 5%, 6%, 7%, 8%, 9%, 10%, 15%, 20%, 25%, or 30% of at least one carbohydrate. A food composition or food product comprising a composition obtained from an in vitro culture of mammary epithelial cells may comprise, by weight, about 1%, 2%, 3%, 4%, 5%, 6%, 7%, 8%, 9%, 10%, 15%, 20%, 25%, or 30%, or a range between any two of the foregoing values, of at least one carbohydrate. A food composition or food product comprising a composition obtained from an in vitro culture of mammary epithelial cells may comprise, by weight, at least about 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, or 3% of at least one mineral. A food composition or food product comprising a composition obtained from an in vitro culture of mammary epithelial cells may comprise, by weight, at most about 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, or 3% of at least one mineral. A food composition or food product comprising a composition obtained from an in vitro culture of mammary epithelial cells may comprise, by weight, about 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, or 3%, or a range between any two of the foregoing values, of at least one mineral. A food composition or food product comprising a composition obtained from an in vitro culture of mammary epithelial cells may comprise, by weight, less than about 0.0005% of at least one mineral. A food composition or food product comprising a composition obtained from an in vitro culture of mammary epithelial cells may be substantially free of an antibody, substantially free of whole cells, and substantially free of bacterial microbes.

A food composition or food product comprising a composition obtained from an in vitro culture of mammary epithelial cells may further comprise a nutritionally beneficial amount of at least one immunoglobulin, such as Immunoglobulin A; probiotic microbes, such Pseudomonas, Staphylococcus, Streptococcus, Variovorax, Bifidobacterium, Flavobacterium, Lactobacillus, Stenotrophomonas, Brevundimonas, Chryseobacterium, and Enterobacter; immune enhancing cells such as leukocytes; or at least one vitamin. A food composition or food product may comprise a cheese, a yogurt, or an infant formula. A food composition or food product may have a variety of nutritional compositions. For example, a food composition or food product may comprise, by weight, about 40% to about 90% water, about 1% to about 2% of at least one protein, about 3% to about 5% of at least one fat, about 7% to about 8% of at least one carbohydrate, and about 0.1% to about 1% of at least one mineral. The mammary epithelial cells from which a food composition or food product may be obtained may be of human origin or may be of the origin of any mammalian species.

### Engineered Cells

Aspects of the present disclosure may comprise an engineered cell exhibiting one or more characteristics of a mammary epithelial cell, the engineered cell comprising an attenuated expression mechanism of a target gene or a target transcript as compared to a natural expression mechanism of a corresponding non-engineered cell. As used herein, the term “expression” used interchangeably with the term “cell expression” and “gene expression” generally refers to the process by which information from a gene is used in the synthesis of a functional gene product. These products may be proteins or may be a functional RNA. Expression may comprise genes translated into mRNA and then translated into protein or genes transcribed into RNA but not translated into protein. Expression may refer to polynucleotides transcribed into mRNA and/or the process by which the transcribed mRNA are subsequently translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” may also refer to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell.

An engineered cell may comprise a cell exhibiting one or more characteristics of a cell of a mammary cell lineage. A mammary cell lineage may display a biomarker typical of the phenotype of a mammary cell linear. A mammary cell lineage biomarker may comprise EpCAM, CK18, CK14, P63, CD49f, K14, K18, SMA, CK18 (associated with luminal cells), CK14 (associated with myoepithelial cells), α-SMA, p63, laminin-1, CD24, CD29, MUC1, or α- and β-casein mRNA. An engineered cell or cell used in aspects of the present disclosure may also comprise a cellular morphology indicative of the mammary cell lineage. An engineered cell or cell used in aspects of the present disclosure may exhibit one or more characteristics of a mammary epithelial cell, a mammary alveolar cell, or a mammary myoepithelial cell. An engineered cell may be capable of lactation. An engineered cell may be configured to generate a milk nutrient in response to a hormone or a signaling factor. An engineered cell may have an altered response to a hormone or signaling factor such as an enhanced or diminished sensitivity, such as to insulin. An engineered cell may be configured to generate a milk component in response to a hormone or signaling factor. An engineered milk-producing cell may comprise an engineered mammary epithelial cell or an engineered mammary alveolar cell. An engineered mill-producing cell may comprise engineering, quantifying, analyzing, or comparing a genome. A genome of an engineered cell may be compared to a control cell. A control cell may comprise a cell corresponding to a non-engineered or non-mammary adult stem cell. A control cell may comprise a corresponding non-engineered mesenchymal stem cell. A naturally occurring milk-producing cell may comprise a mammary alveolar cell.

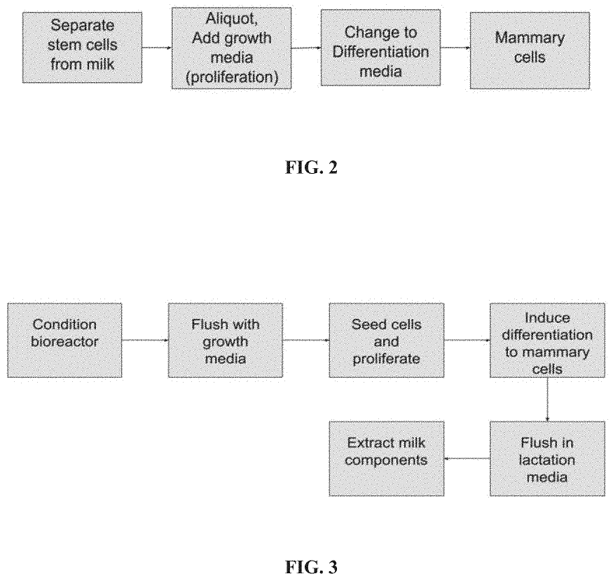

An expression mechanism may comprise an expression profile of a of a target gene, a target transcript, or a target protein. There may be one or there may be a plurality of target genes, transcripts, or proteins. There may be a first target gene, a first target transcript, or a first target protein and a second target gene, a second target transcript, or a second target protein. There may be subsequent target genes, transcripts, or proteins. A target gene or transcript may regulate the production of milk proteins, milk carbohydrates, the degradation of lactose, the production of milk lipids, or the production of milk fats. A modified expression profile may comprise an up-regulated expression of a first target gene, a first target transcript, or a first target protein, and a down-regulated expression of a second target gene, a second target transcript, or a second target protein. As illustrated in FIG. 3, a target gene may be identified through bioinformatics approaches, a database, or gene profiling. An engineering gene vector may be developed from the target gene identification using a specific choice of vector, insertion of the target gene, and choice of promotor, and upon insertion into a cell, genetic manipulation may be acquired wherein gene expression may be up- or down-regulated.

Higher or lower levels of a milk component obtained from the in vitro culture of a mammary cells, or a mammary-like cell can be obtained by engineering stem cells, mammary cells, or mammary-like cells such that the genome of the engineered stem cells, engineered mammary cells, or engineered mammary-like cells is altered, or different, from the genome of the naturally-occurring cells. Various genetic constructs responsible for the expression of a milk component in the liquid composition can be edited, disrupted, silenced, upregulated, or attenuated, etc. using standard molecular biology methods and techniques to produce a modified expression or a modified expression profile of such genetic construct or genetic constructs

The reduction or lowering of a concentration or amount of a milk component in the liquid composition may be desired through engineering of the cells, or through the use of separation or filtration methods. For example, the gene, genetic construct, or expression mechanism, for a certain lipid or fat can be edited, disrupted, silenced, or attenuated, in a cell. In a non-limiting example, at least one lipid or fat may include a long chain fatty acid that is mostly saturated. For example, the triglyceride concentration in the liquid composition can be reduced or substantially eliminated if at least one of the gene(s) encoding the lipids and fatty acids are edited, disrupted, silenced, or attenuated. At least one lipid or milk lipid can be upregulated or downregulated, or attenuated which may comprise at least one of palmitic acid, myristic acid, stearic acid, butyric acid, caproic acid, oleic acid, linoleic acid, α-linoleic acid, vaccenic acid, eicosapentaenoic acid, docosahexaenoic acid, calendic acid, γ-linoleic acid, eicosadienoic acid, dihomo-γ-linoleic acid, arachidonic acid, docosadienoic acid, adrenic acid, asbond acid, tetracosatetraenoic acid, tetracospentaenoic acid, hexadecatrienoic acid, stearidonic acid, eicosatrienoic acid, eicosatetraenoic acid, heneicosapentaenoic acid, docosapentaenoic acid, docosahexaenoic acid, tetracosapentaenoic acid, tetracosahexaenoic acid, rumenic acid, caprylic acid, or capric acid. A gene, genetic construct, or expression mechanism, for a certain protein, e.g., α-casein or β-casein, can be edited, disrupted, silenced, or attenuated, in a cell. For example, a gene, genetic construct, or expression mechanism, for a certain whey protein, e.g., β-lactoglobulin, glycomacropeptide, or lactalbumin, can be edited, disrupted, silenced, or attenuated, in a cell so that the resulting engineered cell expresses substantially no such whey protein.

α-casein production or expression can be enhanced or upregulated in a mammary cell or a mammary-like cell. In such a cell, the use of transcription factors can enhance transcription and increase production of the protein. Inducible promoters can be inserted upstream of the target gene to provide a controllable expression profile for the desired expression product. Such systems can be utilized in conjunction with a cell's endogenous gene(s), or in conjunction with an exogenous transgene in a cell. In bovines, genes known to influence protein content and composition in milk may be the casein genes CSN1 S1, CSN2, CSN1S2, and CSN3, encoding the casein proteins alpha S1 (α_{S1}), beta (β), alpha S2 (α_{S2}), and kappa (κ), respectively. See, Ferretti, L., Leone, P. and Sgaramella, V., 1990. Long range restriction analysis of the bovine casein genes. Nucleic acids research, 18(23), pp. 6829-6833; and Threadgill, D. W. and Womack, J. E., 1990. Genomic analysis of the major bovine milk protein genes. Nucleic acids research, 18(23), pp. 6935-6942, each of which is incorporated herein by reference in their entireties for all purposes. These genes may be located on BTA6 in the so-called casein gene cluster, which spans ˜250 kb, see, for example, Boettcher et al. (2004), entitled “Effects of casein haplotypes on milk production traits in Italian Holstein and Brown Swiss cattle,” Journal of dairy science, 87(12), pp. 4311-4317, which is incorporated herein by reference in its entirety for all purposes. All caseins may account for about 75% of the milk protein content (see, for example, Gallinat, J. L., Qanbari, S., Drögemüller, C., Pimentel, E. C. G., Thaller, G. and Tetens, J., 2013. DNA-based identification of novel bovine casein gene variants. Journal of dairy science, 96(1), pp. 699-709, which is incorporated herein by reference in its entirety for all purposes); the remaining 25% may be whey proteins.

The raising or increasing of a concentration or amount of a milk component in a liquid composition may be desired. For example, the gene, genetic construct, or expression mechanism, for a certain lipid or fat can be upregulated, in a mammary cell, or mammary-like cell. The gene, genetic construct, or expression mechanism, for a certain protein can be upregulated in a mammary cell, or a mammary-like cell. For example, the gene, genetic construct, or expression mechanism, for a certain oligosaccharide can be upregulated, in a mammary cell, or a mammary-like cell. In another example, human mammary cells or human mammary-like cells can be engineered or modulated to upregulate Secretor gene that encodes fucosyltransferase-2 (FUT-2) or the Lewis gene that encodes fucosyltransferase-3 (FUT-3) enzyme expression levels. These genes may be involved in the production of Human Milk Oligosaccharides (HMOs). An upregulation may comprise a gene knock in. As used herein, the term “knock-in” generally refers to the insertion of an exogenous or a heterologous gene into a specific locus in the genome of a cell. The knocked-in exogenous gene can be used to analyze product function and/or expression pattern. The exogenous gene can be introduced to disrupt the endogenous gene, generating a knock-out/knock-in cell. The knock-in gene can be used with an inducible gene expression strategy with, for example, a Cre/loxP system, a ubiquitous gene expression strategy using ROSA26 locus, or other various strategies.

Aspects of the present disclosure may comprise an attenuated expression mechanism which may comprise an altered promoter sequence of a target gene. As used herein, the term “promoter” generally refers to sequences that are DNA sequences that define where transcription of a gene by RNA polymerase begins. Promoter may be located directly upstream or at the 5′ end of the transcription initiation site. Promoters useful for expressing the recombinant genes described herein may include both constitutive and inducible/repressible promoters. Examples of inducible/repressible promoters may include galactose-inducible promoters (e.g., PLAC4-PBI). Where multiple recombinant genes are expressed in an engineered cell, the different genes can be controlled by different promoters or by identical promoters in separate operons, or the expression of two or more genes may be controlled by a single promoter as part of an operon. A promotor may be a DNA sequence to which proteins bind that initiate transcription of RNA from the DNA downstream of it. The RNA may encode a protein or may have a unique function itself such as a mRNA, siRNA, or tRNA. An attenuated expression mechanism may affect an altered signaling pathway in an engineered cell. At least one gene, genetic construct, or expression mechanism responsible for the expression of a particular milk component can be edited using a heterologous polypeptide including a gene regulating moiety, wherein the gene regulating moiety may be configured to regulate an expression of at least one of a target gene, a target transcript, or a target protein of the stem cell, mammary cell, or mammary-like cell or a derivative thereof, thereby effecting a modified expression profile of the at least one of a target gene, the target transcript, or target protein in an engineered cell as compared to a corresponding non-engineered cell.

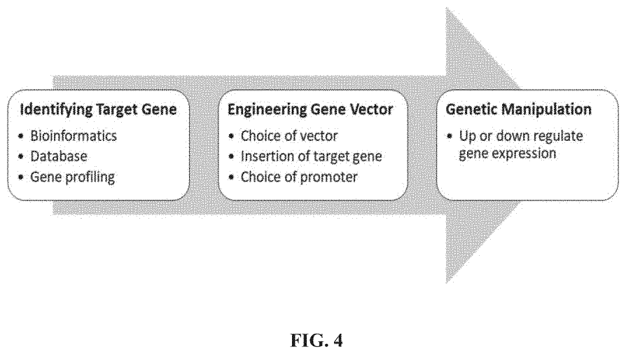

In a mammary cell, or a mammary-like cell, at least one inducible promoter, such as Tet-ON, can be inserted upstream of at least one of the listed genes. Among the various inducible promoters known or described herein, the tetracycline-dependent regulatable gene expression system can be used to induce the expression of casein gene, as a non-limiting example. Tet-regulated gene expression systems may be a widely used gene regulation systems and may comprise of two variants: the Tet-off and the Tet-on systems. Randomly induced mutations of the Tet-OFF-based tetracycline-controlled transactivator (tTA) may lead to the subsequent development of the Tet-ON system. The Tet-ON system may offer many advantages over other regulatable gene expression systems. The Tet-on system in FIG. 4 may show that the presence of doxycycline (Dox) results in its binding to the transactivator rtTA (reverse tetracycline-controlled transactivator) rtTA, and then the rtTA/Dox complex binds to the tetO sequences/Tet Response Element (TRE), allowing promoter activation and casein gene expression. Following introduction of an inducer protein to the thus engineered cell, the inducible promoter may be activated by the inducer protein and transcription of the subject gene may be initiated on demand. Examples of inducible promoter systems suitable for use with the mammary, or mammary-like cells, include and are not limited to Tet-On, ER (Estrogen receptor) system, Cumate-Controlled Operator System, etc. Such inducible promoter systems can also be utilized with exogenous transgenes inserted into the stem cells, mammary cells or mammary-like cells.

An attenuated expression mechanism may be affected by an endonuclease. An endonuclease may be an enzyme that cleaves a phosphodiester bond within a polynucleotide chain. An endonuclease may cleave DNA at a specific site, a restriction site or target sequence. An endonuclease can be an RNA-guided endonuclease. RNA-guided endonucleases may be useful as they can be a clustered regularly interspaced short palindromic repeats (CRISPR)-associated (Cas) endonuclease. Clustered regularly interspaced short palindromic repeats (CRISPR) along with CRISPR-associated (Cas) proteins may comprise a programmable genome-engineering tool that enables easy targeting and manipulation of precise genomic sequences in bacteria, plants, fungi and mammals, including humans. In this system, two small RNAs—the CRISPR RNA (crRNA) may form a complex with a trans-activating CRISPR RNA (tracrRNA) and function together in targeting the nuclease Cas9 to specific DNA sequences, where it can generate a double-stranded DNA break (DSB). The two RNAs can be joined together to form a single guide RNA (sgRNA). RNA can be used to guide Cas9 to specific genomic sequences and thus enable gene editing and genome engineering.

Numerous Cas endonuclease enzymes may various specificities and various advantages in certain circumstances. By way of non-limiting example, such Cas endonucleases suitable for use in editing the genome of a mammary epithelial cell, or a mammary-like cell, may include C2C1, C2C2, C2C3, Cas1, Cas1B, Cas2, Cas3, Cas4, Cas5, Cas5e, Cas6, Cas6e, Cas6f, Cas7, Cas8a, Cas8a1, Cas8a2, Cas8b, Cas8c, Cas9, Cas10, Cas10d, Cas10, Cas10d, Cas11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, or any variants or combinations thereof. Methods, procedures, and techniques, including other reagents, may be suitable for use with the cells, genes and other genetic constructs.

A genome may be edited by using site-directed nucleases to cut deoxyribonucleic acid (DNA) at precise target locations in the genome, thereby creating single-strand or double-strand DNA breaks at particular locations within the genome. Such breaks can be repaired by natural, endogenous cellular processes, such as homology-directed repair (HDR) and non-homologous end joining (NHEJ). See, for example, Cox et al., Nature Medicine 21(2), 121-31 (2015), which is incorporated herein by reference in its entirety for all purposes. These two main DNA repair processes consist of a family of alternative pathways. NHEJ may directly join the DNA ends resulting from a double-strand break, sometimes with the loss or addition of nucleotide sequence, which may disrupt or enhance gene expression. HDR may utilize a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few base pairs flanking the DNA break site to drive a more favored DNA end joining repair outcome, and recent reports have further elucidated the molecular mechanism of this process; see, e.g., Cho and Greenberg, Nature 518, 174-76 (2015); Kent et al., Nature Structural and Molecular Biology, Adv. Online doi:10.1038/nsmb.2961 (2015); Mateos-Gomez et al., Nature 518, 254-57 (2015); Ceccaldi et al., Nature 528, 258-62 (2015); each of which is incorporated herein by reference in their entireties for all purposes.

Genetic modification of engineered cells (e.g., stems cells, mammary cells, or mammary-like cells) can be accomplished by transducing a substantially homogeneous cell (e.g., stems cells, mammary cells, or mammary-like cells) composition with a recombinant DNA or RNA vector construct. As used herein, the term “expression control sequence” or “regulatory sequences” are used interchangeably and generally refer to polynucleotide sequences which may affect the expression of coding sequences to which they are operably linked. Expression control sequences may be sequences which control the transcription, post-transcriptional events, and translation of nucleic acid sequences. Expression control sequences may include appropriate transcription initiation, termination, promoter and enhancer sequences; efficient RNA processing signals, such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences may differ depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

As used herein, the term “regulatory region of a nucleic acid molecule” generally refers to a cis-acting nucleotide sequence that influences expression, positively or negatively, of an operatively linked gene. Regulatory regions may include sequences of nucleotides that confer inducible (e.g., require a substance or stimulus for increased transcription) expression of a gene. When an inducer is present or at increased concentration, gene expression can be increased. Regulatory regions may also include sequences that confer repression of gene expression (e.g., a substance or stimulus decreases transcription). When a repressor is present or at increased concentration gene expression can be decreased. Regulatory regions may influence, modulate or control many in vivo biological activities including cell proliferation, cell growth and death, cell differentiation and immune modulation. Regulatory regions may bind to one or more trans-acting proteins, which may result in either increased or decreased transcription of the gene.

Particular examples of gene regulatory regions are promoters and enhancers. Promoters are sequences located around the transcription or translation start site, for example, positioned 5′ of the translation start site. Promoters may be located within 1 Kb of the translation start site, but can be located further away, for example, 2 Kb, 3 Kb, 4 Kb, 5 Kb or more, up to and including 10 Kb Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions may also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

A vector can be a retroviral vector (e.g., gamma retroviral), which is employed for the introduction of the DNA or RNA construct into the host cell genome. For example, a polynucleotide encoding a desired polypeptide can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

As used herein, the term “vector” generally refers to a nucleic acid molecule capable of transporting another nucleic acid to which it has been linked. A vector may comprise a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors may include cosmids, bacterial artificial chromosomes (BAC) and yeast artificial chromosomes (YAC). A vector may comprise a viral vector, wherein additional DNA segments may be ligated into the viral genome. Some vectors are capable of autonomous replication in a host cell into which they are introduced (e.g., vectors having an origin of replication which functions in the host cell). Other vectors can be integrated into the genome of a host cell upon introduction into the host cell and are thereby replicated along with the host genome. Some vectors are capable of directing the expression of genes to which they are operatively linked. For initial genetic modification of the cells to provide the desired polypeptide expressing cells, a retroviral vector may be employed for transduction, however any other suitable viral vector or non-viral delivery system can be used. For subsequent genetic modification of the cells, retroviral gene transfer (transduction) can likewise prove effective. Combinations of retroviral vector and an appropriate packaging line are also suitable, where the capsid proteins will be functional for infecting mammalian cells.

Transducing viral vectors can be used to express a desired polypeptide in an engineered mammary, or mammary-like cell. For example, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649, 1997; Naldini et al., Science 272:263 267 (1996); and Miyoshi et al., Proc. Natl. Acad. Sci. U.S.A. 94: 10319, (1997)), each of which is incorporated herein by reference in their entireties for all purposes. Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244: 1275-1281 (1989); Eglitis et al., BioTechniques 6:608-614, (1988); Tolstoshev et al., Current Opinion in Biotechnology 1:55-61 (1990); Sharp, The Lancet 337: 1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)), each of which is incorporated herein by reference in their entireties for all purposes. Retroviral vectors are particularly well developed and have been used in clinical settings (see, e.g., Rosenberg et al., N Engl. J. Med 323:370 (1990); Anderson et al., U.S. Pat. No. 5,399,346); each of which is incorporated herein by reference in their entireties for all purposes.

A viral construct such as a retroviral construct may include at least one transcriptional promoter/enhancer or locus defining element(s), or other elements that control gene expression by other approaches, such as alternate splicing, nuclear RNA export, or post-translational modification of messenger. A vector construct may further comprise a packaging signal, long terminal repeats (LTRs) or portions thereof, or positive and negative strand primer binding sites appropriate to the virus used. A construct may also include a signal sequence for secretion of the peptide from a host cell in which it is placed. A signal sequence may comprise a mammalian signal. Other non-viral vectors can be used such as cationic lipids, polylysine, or dendrimers. An expression construct may comprise the necessary elements for the transcription and translation of an inserted coding sequence. An expression construct may further comprise sequences engineered to enhance stability, production, purification, or yield of the expressed peptide. Prokaryotic or eukaryotic cells can be used as host-expression systems to express polypeptides of interest such as microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus (CaMV); tobacco mosaic virus (TMV)) or transformed with recombinant plasmid expression vectors, such as Ti plasmid, containing the coding sequence. Mammalian expression systems can also be used to express polypeptides of interest. Aspects of the present disclosure may comprise an engineered cell exhibiting one or more characteristics of a mammary epithelial cell, the engineered cell comprising an inducible gene expression system, the inducible expression system including an exogenous nucleic acid, wherein the inducible gene expression system may be configured to express a hormone or a signaling factor. An inducible gene expression system may be configured to inducibly express a hormone or a signaling factor. Such a hormone or signaling factor may comprise insulin. Such a hormone or signaling factor may comprise estrogen. Such a hormone or signaling factor may comprise progesterone. Such a hormone or signaling factor may comprise prolactin.

Forced, transient, non-integrative gene expression can be achieved using various nucleic acid molecules such as messenger ribonucleic acid (mRNA), complementary deoxyribonucleic acid (cDNA), micro RNA (miRNA), transfer RNA (tRNA), silencing RNA (siRNA) or any variants, combinations, or analogs thereof. A nucleic acid may be natural in origin or may be a synthetic nucleic acid molecule. Gene expression may be transient, non-integrative such that nucleic acid molecules delivered into a cell are not integrated into the genome of the cell. mRNA introduced into a cell may make a protein by translation which may be sufficient to differentiate a naïve stem cell or progenitor cell into a mature cell type. mRNA differentiation protocols may be short (e.g., up to 14 days) and may not cause or harbor adverse effects since mRNAs are otherwise degraded and do not integrate with the host cell genome. mRNA may be a single stranded RNA molecule that corresponds to the genetic sequence of a gene and may be read by the ribosome in the process of transcription. mRNA may be complementary to one of the DNA strands of a gene. An mRNA molecule may carry a portion of the DNA code to other parts of the cell for processing. mRNA may be created during transcription wherein a single strand of DNA is decoded by RNA polymerase, synthesizing mRNA.

siRNA may be a class of short, double stranded RNA non-coding RNA molecules which may interfere with the expression of specific genes with complementary nucleotide sequences. siRNA may interfere with gene expression by degrading mRNA after transcription, preventing translation. siRNAs may be 20-24 base pairs in length with phosphorylated 5′ ends and hydroxylated 3′ ends. siRNAs may target complementary mRNA for degradation, thus preventing translation. siRNA molecules may be associated with RNA interference or gene silencing, also called gene knockdown. Various methods for gene silencing may include the use of antisense oligonucleotides, ribozymes, and RNA interference. The term RNA interference (RNAi) may describe a cellular mechanism that uses the gene's own DNA sequence to attenuate or modulate the expression of the gene. In mammals, RNAi may be triggered by double-stranded RNA (dsRNA). During RNAi, long dsRNA may be cut or diced into small fragments ˜21-25 nucleotides long by an enzyme called “Dicer.” These small fragments, referred to as small interfering RNAs (siRNA), may bind to proteins Argonaute proteins. After binding to an Argonaute protein, one strand of the dsRNA may be removed, leaving the remaining strand available to bind to messenger RNA target sequences according to the rules of base pairing: A binds U, G binds C, and vice versa. Once bound, the Argonaute protein can either cleave the messenger RNA, destroying it, or recruit accessory factors to regulate the target sequence in other ways.

siRNA molecules may be designed for targeting a specific gene to silence or attenuate expression of a milk component. As used herein, the term “gene silencing” generally refers to the regulation of gene expression in a cell to prevent the expression of a certain gene. Gene silencing can occur during either transcription or translation. When genes are silenced, their expression may be reduced. In contrast, when genes are knocked out, they may be completely erased from the organism's genome and, thus, have no expression. Gene silencing may be considered a gene knockdown mechanism since the methods used to silence genes, such as RNAi, CRISPR, or siRNA, generally reduce the expression of a gene by at least approximately 70% but may not completely eliminate it.

Numerous methods may exist for designing and chemically synthesizing high targeting short (e.g., about 18 to about 30 nucleotides, about 21-30 nucleotides, or about 21 to about 25 nucleotides) molecules. Designing and chemically synthesizing short molecules may comprise less than 18 nucleotides or may comprise longer than 25 nucleotides. Various software systems have been developed that can design specific siRNA molecules. See, for example Naito, Yuki, and Kumiko Ui-Tei, “siRNA Design Software for a Target Gene-Specific RNA Interference,” Frontiers in Genetics vol. 3 102. 11 Jun. 2012, doi:10.3389/fgene.2012.00102, which is incorporated herein by reference in its entirety for all purposes. Several reviews cover the art of designing effective siRNAs, including Davidson, B L, McCray Jr., P B, “Current prospects for RNA interference-based therapies,” Nat Rev Genet, 12:329-40, 2011; Peek, A S, Behlke, M A, “Design of active small interfering RNAs,” Curr Opin Mol Ther, 9:110-8, 2007; and Birmingham, A, et al., “A protocol for designing siRNAs with high functionality and specificity,” Nat Protocols, 2[9]:2068-78, 2007; each of which is incorporated herein by reference in their entireties for all purposes. Numerous online tools may be available, as well, including from IDT, Life Technologies, and Thermo Fisher Scientific.

Micro RNA (miRNA) can be small non-coding RNA molecules that function in RNA silencing and post-transcriptional regulation of gene expression. miRNAs base-pair with complementary sequences within mRNA molecules, silencing the mRNA molecules. Silencing may be achieved upon binding of the miRNA to the 3′UTR of the target mRNA through cleavage of the mRNA strand into two pieces, destabilization of mRNA through shortening the poly-A tail, or through inefficient translation of the mRNA into proteins by ribosomes.

Transfer RNA (tRNAs) are adaptor molecules important to translation composed of RNA which serve as a physical link between an mRNA and an amino acid sequence of proteins by carrying an amino acid to the ribosome as directed by a 3-nucleotide codon in a mRNA. tRNAs may be essential for the initiation of protein synthesis by catalyzing ligation of each amino acid to its cognate tRNAs. The translational functions of these entities may be necessary for myogenesis and myogenic differentiation/proliferation. tRNAs that may modulate myogenic gene expression may comprise leucyl-tRNA synthetase, the tRNA gene for lysine, or the tRNA gene for phenylalanine.

cDNA may be a DNA copy synthesized from a single-stranded RNA molecule such as mRNA or miRNA, and produced by reverse transcriptase, a DNA polymer that can use either DNA or RNA as a template. A cDNA can be delivered (e.g., transfected) into a cell to transfer the cDNA that codes for a protein of interest to the recipient cell. A nucleic acid molecule may be delivered to a cell or stem cell to modulate expression of one or more genes in the cells. The modulation may be in a transient and non-integrative manner such that the nucleic acid molecules are not integrated into a genome of the cells. Progenitor cells may be generated following delivery of the cDNA molecules.

It may be desirable to alter, silence, attenuate, or knock-out a gene or genetic construct in order to modulate the expression or expression profile of a milk component. As used herein, the term “knock-out” generally refers to a gene whose level of expression or activity has been reduced to zero. In some instances, a gene can be knocked-out via deletion of some or all of its coding sequence. In other instances, a gene can be knocked-out via introduction of one or more nucleotides into its open reading frame, which results in translation of a non-sense or otherwise non-functional protein product. For example, the cells (e.g., stems cells, mammary cells, or mammary-like cells), may have the expression mechanism for at least one protein, at least one fat, an oligosaccharide, or at least one amino acid, altered, silenced, attenuated, or knocked-out. Alternatively, or additionally, the gene, genetic construct or expression mechanism for at least one of the following genes can be readily altered, silenced, attenuated, or knocked-out using known methods and reagents, including those described herein. Such genes, genetic constructs or expression mechanisms include, but are not limited to, sirtuin (SIRT1), lactose, protein tyrosine phosphatase enzyme-1B (PTP-1B), protein tyrosine phosphatase enzyme-RF (PTP-RF), USF, GATA-1, GATA-2, GATA-3 c-Myc:Max, ATF, USF, CREB, TATA, SREBP-1, Arnt, USF, Tal-1beta, NRF-2, FOXD3, HNF-3beta, v-Myb, FOXJ2, FOXO1, Evi-1, FOXO4, ARP-1, Staf, NF-kappaB, myogenin, AML-1a, Elk-1 Oct-1, Tax/CREB, HNF-1, AP-1, Ahr, Bachl, RP58, AREB6, NKX3A, XFD-1, deltaEF1, poly A downstream element, Pax-4, Sox-5, Sox-9, Zic3, E47, LPL, AGPAT6, CD36, SCD, GPAM, BTN1A1, ACACA, ACSL1, LPIN1, FASN, FABP3, AGPAT6, SREBP1 (see e.g., J. S. Osorio et al. Physiol Genomics 48: 231-256, 2016, which is incorporated herein by reference in its entirety for all purposes), EpCAM, CK18, CK14, P63, CD49f, K14, K18, SMA, CK18 (associated with luminal cells), CK14 (associated with myoepithelial cells), α-SMA, p63, laminin-1, CD24, CD29, MUC1, and α- and β-casein mRNA.

Certain target genes or target factors may be altered, attenuated, upregulated for specific purposes and intended resulting product compositions. Aspects of the present disclosure may comprise an engineered cell wherein the target gene or target transcript regulates production of a monosaccharide, a disaccharide, or an oligosaccharide. A disaccharide may comprise lactose. An engineered cell may comprise the target gene or target transcript lactase (LCT). A target gene or target transcript may be endogenous to the corresponding non-engineered cell or may not be endogenous to the corresponding non-engineered cell.

The following is a non-limiting list of various genes, and their corresponding function, that can have their expression profile altered in a stem cell, a mammary cell, or a mammary-like cell as compared to their expression profile in a non-engineered cell: CIDEA, transcriptional co-activator for XOR; XDH, produces XOR, for the formation of milk fat globule membrane; C/EBPβ, transcriptional activator; HDAC, negative regulator of milk fat globule proteins; DGAT1&2, triacylglycerol biosynthesis; acyl-CoA: cholesterol acyltransferase, production of milk cholesterol; AKT1, lactation initiation; S14, de novo lipid synthesis8; LPL, produces long chain fatty acid for milk fat synthesis; BTN/BTN1A1, milk lipid secretion; ADFP, formation of milk fat globule; ACACA/B, milk lipid production; FASN, de novo fatty acid synthesis; ACLY, fatty acid biosynthesis; S-acyl fatty acid synthase thioesterase, medium chain/OLAH, production of medium chain fatty acids; ACOT, production of fatty acids; SCD, synthesis of unsaturated fatty acids; ELOVL, synthesis of long chain fatty acids; FADS, synthesis of unsaturated fatty acids; ACSS, synthesis of short chain fatty acids; ACSL, synthesis of long chain fatty acids; GPD1L, de novo fatty acid synthesis; GK5, de novo fatty acid synthesis; DGKA, de novo fatty acid synthesis; GPAM, Triacylglycerol biosynthesis; AGPAT1, Triacylglycerol biosynthesis; LPIN1/2, triglyceride biosynthesis; SLC27A, synthesis of long chain fatty acids; FABP, fatty acid transport; ABCG2, cholesterol transport; FDFT1, cholesterol synthesis and metabolism; SC4MOL, cholesterol synthesis and metabolism; SC5DL, cholesterol synthesis and metabolism; IDI1, cholesterol synthesis and metabolism; MVD, cholesterol synthesis and metabolism; CH25H, cholesterol synthesis and metabolism; DHCR7, cholesterol synthesis and metabolism; PLIN2, lipid droplet synthesis; SREBP1, regulator of lipid synthesis; MUC1, milk fat globule protein; MFGE8, milk fat globule protein; mTORC1, regulation of protein synthesis; CASTOR1, arginine/amino acid sensing; GATOR1, lysosomal regulation; GATOR2, regulation of protein synthesis; RRAGA/B, regulation of protein synthesis; RHEB, regulation of protein synthesis; TSC2, regulation of protein synthesis; PIK3C3, regulation of protein synthesis; EAAT3, amino acid transporter; CAT1, amino acid transporter; 4F2hc, amino acid transporter; LAT1, amino acid transporter; ASCT2, amino acid transporter; AMPK, metabolic regulation; STK11, energy homeostasis; SIRT1, energy homeostasis; ARNTL, molecular clock; CLOCK, molecular clock; NAMPT, metabolic regulation; CRY, regulation of lipid and protein biosynthesis; PER1, regulation of lipid and protein biosynthesis; CSN1, produces α S1/S2 casein; CSN2, produces 13 casein; CSN3, produces κ casein; LALBA, produces α-Lactalbumin; LTF, produces lactoferrin; LYZ, produces lysozyme; LPO, produces lactoperoxidase; TC-1, produces haptocorrin; SPP1, produces osteopontin; ABL, produces serum albumin; C3, produces complement C3; C4, produces complement C4; Galactosyltransferase; B4GALT1-2, sugar biosynthesis; LGALS1-3, 8-9, 12-14, 16, regulation of milk production; LCT, cystine transporter; GLUT1, glucose transport; FUT1-9, adds a fucosyl group onto milk oligosaccharides; Sialyltransferases, adds a sialyl group onto milk oligosaccharides; GALM, hexose metabolism; GLTP, transport of glycolipids; LGALSL, regulation of milk production; N-acetylglucosaminyltransferase, sugar biosynthesis; and/or glycosyltransferase, sugar biosynthesis.

Any of the above-listed milk component genes or proteins can be targeted using gene silencing methods. Using the sequence of the to-be-silenced gene, designing the siRNA may comprise the use of rational design software systems described above. All known sequences for the genes and expression products listed above and for other milk components are incorporated herein by reference for all purposes. Aspects of the present disclosure may comprise an engineered mammalian cell exhibiting one or more characteristics of a mammary epithelial cell, the engineered cell comprising, in its genome, an exogenous nucleic acid, wherein the exogenous nucleic acid encodes a hormone or a signaling factor. A hormone or signaling factor may be a growth factor, glucocorticoid, insulin, progesterone, prolactin, or estrogen. An engineered cell may exhibit one or more characteristics of a mammary epithelial cell, the engineered cell comprising at least one exogenous nucleic acid, wherein the at least one exogenous nucleic acid may encode at least one of a glucocorticoid, insulin, transferrin, apo transferrin, progesterone, prolactin, ethanolamine, estrogen, fibroblast growth factor (FGF), FGF receptor, TBX3, NRG3/ERBB4, Wnt/LEF1, insulin-like growth factor 1 (IGF-1), epidermal growth factor (EGF), hepatocyte growth factor (HGF), nuclear factor κB (NF-κB), pTHrP, non-phospho β-catenin, p-p65, RELA, CTNNB1, SMAD3, beclomethasone, betamethasone, budesonide, cortisone, dexamethasone, hydrocortisone, methylprednisolone, prednisolone, prednisone, or triamcinolone. An exogenous nucleic acid may comprise a heterologous nucleic acid or a homologous nucleic acid. An exogenous nucleic acid may be a nucleic acid originating outside the organism of interest. Exogenous nucleic acids may be introduced into a cell or organism through transformation or transfection either naturally or artificially. As used herein, the term “transfect” or “transfection” generally refers to the introduction of a heterologous nucleic acid into eukaryote cells, both higher and lower eukaryote cells, as well as yeast and fungal cells. Transfection deliberately introduces nucleic acids into eukaryotic cells artificially to enable the expression or production of proteins using the cell's own machinery or to down-regulate the production of a specific protein by stopping translation.

The stem cells, mammary cells, or a mammary-like cells can be engineered such that the genome of the engineered stem cells, engineered mammary cells, or engineered mammary-like cells is altered, or different, from the genome of the respective naturally occurring cells. Such engineered cells can have genes upregulated to increase cell proliferation. Such engineered cells can have their sensitivity to different growth factors increased or enhanced by upregulating a corresponding receptor in the cell. Upregulation can be achieved through various known method(s) or mechanism(s), including those described herein, and can include epigenetic remodeling, changing of regulatory sequences, removing regulatory molecules, substitution of promoter elements, increasing copy number of genes, or turning genes constitutively on or off. Aspects of the present disclosure may comprise an engineered cell exhibiting one or more characteristics of a mammary epithelial cell, the engineered cell comprising an altered genome that effects an enhancement in cellular sensitivity to insulin of the engineered cell as compared to a cellular sensitivity to insulin of a corresponding non-engineered cell. An altered genome may comprise an altered protein tyrosine phosphatase 1B (PTP1B) gene expression mechanism. An altered genome may comprise an altered protein tyrosine phosphatase receptor type F (PTPRF) gene expression mechanism. An altered genome may comprise an altered sirtuin 1 (SIRT1) gene expression mechanism. An altered SIRT1 gene expression mechanism may comprise an altered SIRT1 regulatory pathway that comprises an exogenous inducible promoter.

Upregulation of a corresponding receptor in a cell may alter the sensitivity to different growth factors by increasing or enhancing them. An altered SIRT1 gene expression mechanism may be configured to overexpress SIRT1. For example, at least one sirtuin (e.g., SIRT1, 3 or 7) can be upregulated to enhance mammary epithelial cell growth and survival, insulin receptor (INSR) can be upregulated to further sensitize the cell for insulin, EGFR can be upregulated to further sensitize the cell for EGF, prolactin receptor (PRLR) can be upregulated to further sensitize the cell for prolactin. Upregulation of lactose synthesis can be done through the upregulation of at least one of AKT1, GLUT1 and lactose synthase (α-lactalbumin and β1,4-galactosyltransferase) genes to increase glucose influx and conversion of glucose to lactose within the cells.

In addition to the specific receptors for culture or lactation medium components (such as hormones), various other genes involved in signaling pathways can be upregulated. In mammary epithelial cells, the Signal Transducer and Activator of Transcription 5A and 5B, also known as STAT5A/B (STAT5) pathway modulates three different cellular outcomes: differentiation, survival, and proliferation. STAT5A and STAT5B, collectively referred to as STAT5A/5B, may be two highly conserved transcription factors activated by various hormones or cytokines—including prolactin, growth hormone, and EGF—that may play important roles in the development and function of mammary glands. STAT5 expression and activity may be regulated by prolactin signaling with JAK2/ELF5, EGF signaling networks that include c-Src, and growth hormone, insulin growth factor, estrogen, and progesterone signaling pathways. Upregulation of STAT5 expression can be achieved through the manipulation of any of these pathways using methods and materials described herein or well known in the art. Activation levels of STAT5 may be influenced by AKT, caveolin, PIKE-A, Pak1, c-Myb, Brk, beta-integrin, dystroglycan, other STATs, and STAT pathway molecules JAK1, Shp2, and SOCS. See for example, Furth, P. A., Nakles, R. E., Millman, S. et al. “Signal transducer and activator of transcription 5 as a key signaling pathway in normal mammary gland developmental biology and breast cancer.” Breast Cancer Res 13, 220 (2011). https://doi.org/10.1186/bcr2921, which is incorporated herein by reference in its entirety for all purposes.

Aspects of the present disclosure may comprise a cell culture comprising an effective amount of a reagent, wherein the reagent is capable of regulating a cellular response to a hormone or a signaling factor of cells in the cell culture. A reagent may comprise a small molecule, a small interfering ribonucleotide (siRNA), a peptide, a nucleic acid, or a transcription factor. A reagent may be capable of enhancing a cellular response to a hormone or growth factor, such as insulin of the cells in the cell culture. The reagent may be capable of modulating the biological activity of at least one of sirtuin 1 (SIRT1), protein tyrosine phosphatase 1B (PTP1B), and protein tyrosine phosphatase receptor type F (PTPRF) of the cells comprised in the cell culture. A reagent may be capable of enhancing the biological activity of sirtuin 1 (SIRT1) of cells in the cell culture. The reagent may comprise resveratrol.

A gene or transcript may regulate the cell response to hormones or signaling factors. Genes or transcripts that may regulate a cell response to hormones or signaling factors may comprise, for example, Sirtuin 1 (SIRT1), Protein Tyrosine Phosphatase 1B (PTP1B), or Protein Tyrosine Phosphatase Receptor Type F (PTPRF). An exogenous nucleic acid may regulate the cell response to hormones or signaling factors. An exogenous nucleic acid may encode a desired polypeptide. A stem or mammary cell can be engineered such that its genome is altered to attenuate the expression of a protein, a fat, or an oligosaccharide. The engineered cell may include an exogenous nucleic acid encoding a hormone or a signaling factor. An engineered cell may exhibit or more characteristics of a mammary epithelial cell, the engineered cell comprising an inducible gene expression system, the inducible gene expression system including an exogenous nucleic acid, wherein the inducible gene expression system may be configured to express a hormone or a signaling factor. At least one exogenous nucleic acid may encode at least one of a hormone or a signaling factor, including, but not limited to, glucocorticoid, insulin, transferrin, apo transferrin, progesterone, prolactin, ethanolamine, estrogen, fibroblast growth factor (FGF), FGF receptor, TBX3, NRG3/ERBB4, Wnt/LEF1, insulin-like growth factor I (IGF-1), epidermal growth factor (EGF), hepatocyte growth factor (HGF), nuclear factor κB (NF-κB), pTHrP, non-phospho β-catenin, p-p65, REA, CTNNB1, or SMAD3. A glucocorticoid may include at least one of a beclomethasone, betamethasone, budesonide, cortisone, dexamethasone, hydrocortisone, methylprednisolone, prednisolone, prednisone, or triamcinolone.

A cell may be engineered with attenuated gene, transcript, or protein expression. A modified expression profile may comprise an up-regulated expression of a gene or a transcript that regulates production of a milk fat. A modified expression profile may comprise a down-regulated expression of a gene or a transcript that regulates production of a milk fat. For example, a cell may be engineered with attenuated expression of cell death-inducing DFFA-like effector A. Adequate lipid secretion by mammary cells during lactation is important for the survival of mammalian offspring and influences the taste and nutritional content of milk. Blood concentrations of non-esterified fatty acids may represent a source of fatty acids for milk fat synthesis and increased milk fat content. Cell death-inducing DFFA-like effector A (CIDEA) is a lipid droplet coat protein which may play a role in the regulation of milk fat synthesis and secretion. CIDEA mRNA may be a mammary gene expressed during the onset of lactation and its inhibition may result in reduced milk lipids. CIDEA may interact with transcription factor CCAAT/enhancer-binding protein β (C/EBPβ). C/EBPβ may play a role in mammary gland development and may regulate mammary stem cells (MaSCs). C/EBPβ may aid in the development of luminal progenitors and increase differentiated luminal cells. C/EBPβ may be a regulator of MaSC repopulation activity and luminal cell lineage commitment. An engineered cell may express attenuated C/EBPβ. Xanthine oxireductase (XOR) may be necessary for milk fat droplet envelopment and secretion during lactation. XOR may be expressed in high levels in the mammary epithelium during lactation. XOR may have two convertible forms: xanthine dehydrogenase (XDH) which uses NAD+ as a cofactor and xanthine oxidase (XO) which uses oxygen. Deficiencies in XOR and its convertible forms may result in xanthinuria and associated lactation problems as well as collapsed mammary epithelium resulting in premature involution of the mammary gland. An engineered cell may express attenuated XOR, XDH, or XO. An engineered cell may express attenuated histone deacetylase (HDAC). HDAC may regulate lipid accumulation. HDAC may decrease histone acetylation and transcription which may suppress lactation. An engineered cell may express attenuated diacylglycerol O-Acyltransferase 1 (DGAT1) or diacylglycerol O-Acyltransferase 2 (DGAT2). DGAT1 may have an effect on milk production ilk fat content, protein yield, total energy excreted, lactose content, and lactose yield. DGAT1 may vary throughout lactation to influence amount and composition of milk. There may be an inverse relationship between fat content and lactose content in milk regulated through DGAT1. DGAT1 may mediate triglyceride synthesis. Attenuation of DGAT1 expression may result in altered nutritional and mineral content in milk. DGAT2 may regulate lipid storage and synthesis. DGAT2 may impact milk yield in cattle and may determine dietary fat uptake and triglyceride synthesis and storage in mammals. An engineered gene may express attenuated acyl-coenzyme A: cholesterol acyltransferase (ACAT). ACAT may catalyze the esterification of free cholesterol which may be required for the secretion of cholesterol and cholesterol esters. ACAT may increase expression during lactation to regulate cholesterol secretion from the liver with triglycerides and alter the milk fat composition. Serine-threonine kinase 1 (AKT1) expression may be attenuated in engineered cells. AKT1 expression may be upregulated during lactation and may induce autocrine prolactin production to initiate lactation. AKT1 expression may induce terminal mammary epithelial differentiation accompanies by milk synthesis even in the absence of lobuloalveolar development. Spot 14 (S14) expression may be attenuated in engineered cells. S14 may be required for de novo lipid synthesis in the lactating mammary gland. S14 may impact triglyceride content and medium chain fatty acids in the triglyceride pool by regulating de novo lipid synthesis.

A gene or transcript regulating the production of milk fat may comprise a gene or a transcript selected from the group consisting of: CIDEA, XDH, C/EBPβ, HDAC, DGAT1, DGAT2, acyl-CoA: cholesterol acyltransferase, AKT1, S14, LPL, BTN/BTN1A1, ADFP, ACACA/B, FASN, ACLY, S-acyl fatty acid synthase thioesterase, medium chain/OLAH, ACOT, SCD, ELOVL, FADS, ACSS, ACSL, GDP1L, GK5, DGKA, GPAM, AGPAT1, LPINL LPIN2, SLC27A, FABP, ABCG2, FDFT1, SC4MOL, SC5DL, IDI1, MVD, CH25H, DHCR7, PLIN2, SREBP1, MUC1, MFGE8, or any variant or combination thereof.

A modified expression profile may comprise an up-regulated expression of a gene or a transcript that regulates production of a milk lipid. A modified expression profile may comprise a down-regulated expression of a gene or a transcript that regulates production of a milk lipid. A gene or transcript which regulates production of a milk lipid may be selected from the group consisting of: palmitic acid, myristic acid, stearic acid, butyric acid, caproic acid, oleic acid, linoleic acid, α-linoleic acid, vaccenic acid, eicosapentaenoic acid, docosahexaenoic acid, calendic acid, γ-linoleic acid, eicosadienoic acid, dihomo-γ-linoleic acid, arachidonic acid, docosadienoic acid, adrenic acid, asbond acid, tetracosatetraenoic acid, tetracospentaenoic acid, hexadecatrienoic acid, stearidonic acid, eicosatrienoic acid, eicosatetraenoic acid, heneicosapentaenoic acid, docosapentaenoic acid, docosahexaenoic acid, tetracosapentaenoic acid, tetracosahexaenoic acid, rumenic acid, caprylic acid, capric acid, or any variant or combination thereof. A gene or transcript regulating production of a milk lipid may comprise a gene or a transcript selected from the group consisting of: CIDEA, XDH, C/EBPβ, HDAC, DGAT1, DGAT2, acyl-CoA: cholesterol acyltransferase, AKT1, S14, LPL, BTN/BTN1A1, ADFP, ACACA/B, FASN, ACLY, S-acyl fatty acid synthase thioesterase, medium chain/OLAH, ACOT, SCD, ELOVL, FADS, ACSS, ACSL, GDP1L, GK5, DGKA, GPAM, AGPAT1, LPINL LPIN2, SLC27A, FABP, ABCG2, FDFT1, SC4MOL, SC5DL, IDI1, MVD, CH25H, DHCR7, PLIN2, SREBP1, MUC1, MFGE8, or any variant or combination thereof.

An engineered cell may regulate milk carbohydrate content. Carbohydrate content may be regulated through a variety of genes. A modified expression profile may comprise an up-regulated expression of a gene or a transcript that regulates production of a milk carbohydrate. A modified expression profile may comprise a down-regulated expression of gene or a transcript that regulates production of a milk carbohydrate. A gene or transcript may regulate production of a milk carbohydrate. A milk carbohydrate may comprise 2′-fucosyllactose, 3-fucosyllactose, difucosyllactose, difucosyllacto-N-tetrose (DFLNT), difucosyllacto-N-hexaose, fucosyllacto-N-hexaose (FLNH), lacto-N-fucopentaose (LNFP) I, LNFP II, LNFP III, 3′-sialyllactose, 6′-sialyllactose, disialyllacto-N-hexaose (DSLNH), disialyllacto-N-tetraose (DSLNT), fucodisialyllacto-N-hexaose (FDSLNH), sialyl-lacto-N-tetraose b (LSTb), and sialyl-lacto-N-tetraose c (LSTc), lacto-N-hexaose, lacto-N-neotetraose (LNnT), lacto-N-tetrose (LNT), lactose, or any variant or combination thereof. Carbohydrate content may be regulated through alpha lactalbumin (LALBA), β-1,4-galactosyltransferase (B4GALT), lectin galactoside-binding soluble (LGALS), lactase (LCT), glucose transporter (GLUT), fucosyltransferase (FUT), sialyltransferase, galactose mutarotase (GALM), glycolipid transfer protein (GLTP), lectin galactoside-binding-like protein (LGALSL), N-acetylglucosaminyltransferase, glycosyltransferase, or any combination or variant thereof.

Alpha lactalbumin (LALBA) may comprise a protein component of the whey fraction of milk. LALBA may regulate the production of lactose in milk. LALBA may be upregulated to increase the production of lactose. LALBA may comprise part of the lactose synthase complex required for lactose formation which may drive milk volume. LALBA may be a source of bioactive peptides and essential amino acids such as tryptophan, lysine, branched-chain amino acids, and sulfur-containing amino acids. β-1,4-galactosyltransferase (B4GALT) may comprise a gene that may encode type II membrane-bound glycoproteins and may transfer galactose in a beta 1,4 linkage to similar acceptor sugars. B4GALT may comprise encode an enzyme which may participate in glycoconjugate and lactose biosynthesis. B4GALT may add galactose to N-acetylglucosaminyltransferase residues that are either monosaccharides or the nonreducing ends of glycoprotein carbohydrate chains. N-acetylglucosaminyltransferase may comprise a key enzyme in glycoprotein biosynthesis. N-glycans on lactoferrin may be released by the activity of endo-N-acetylglucosaminidases and can serve as carbon sources for growth. The degraded N-glycans can be detected in the gut of breast-fed infants which may be correlated with the abundance of Bifidobacterium spp. in the gut. N-acetylglucosaminidase-like enzymes may be from Lactobacillus spp. Milk N-glycans may be upregulated in the later stages of lactation. Milk protein N-glycans may affect gut bacteria such as Lactobacillales and Bacteroidales. Lectin galactoside-binding soluble (LGALS) may encode for a member of the galectin family of carbohydrate binding proteins. Galectins may comprise milk glycan receptors which may affect milk glycans which contain lactose at their reducing end and may be modified to contain N-acetylglucosamine (GlcNAc), galactose (Gal), fucose (Fuc) and/or sialic acid (as N-acetylneuraminic acid; Neu5Ac). Galectin may comprise a major component of milk. Milk glycans may function as prebiotics that help shape the gut microflora, glycan receptor decoys against pathogenic microbes, regulators of immune responses and even regulators of gene expression in intestinal epithelial cell cultures as well as other cell types. Lactase (LCT) may comprise an enzyme that catalyzes the hydrolysis of lactose to glucose and galactose. Lactase is essential to the digestion of milk by breaking down lactose. LCT may comprise a part of the β-galactosidase family of enzymes and may be a glycoside hydrolase involved in the hydrolysis of the disaccharide lactose into constituent galactose and glucose monomers. A modified expression profile may comprise an up-regulated expression of a gene or a transcript that degrades lactose. A gene or transcript which may degrade lactose may comprise a gene or a transcript selected from the group consisting of a Lac operon, lactase, glycosidase, or any combination or variant thereof. Glucose transporters (GLUT) may comprise a group of membrane proteins which facilitate the transport of glucose across the plasma membrane. Glucose is the primary precursor for the synthesis of lactose which may control milk volume by maintaining the osmolarity of milk. Glucose uptake in the mammary gland may play a role in milk production. Milk may be rich with fucosylated oligosaccharides and other glycoconjugates. Fucosyltransferase (FUT) may be involved with the biosynthesis and degradation of oligosaccharides and glycoconjugates. FUT may be upregulated during lactation, particularly early lactation, and may affect fucosyloligosaccharide content in milk. Milk oligosaccharides may influence the composition of intestinal microbiota. The main carbohydrates added to the lactose core may be fucose and the sialic acid (Sia) N-acetylneuraminic acid. Approximately 70% and 28% of human milk oligosaccharides may be fucosylated and sialylated, respectively. A milk oligosaccharide may comprise sialyllactose. Sialyltransferases may be involved in the sialylation of milk oligosaccharides. Sialyltransferase may comprise disulfide-containing, type II transmembrane glycoproteins that catalyze the transfer of sialic acid to proteins and lipids and participate in the synthesis of the core structure oligosaccharides of human milk. Galactose may be metabolized from lactose may enter glycolysis by conversion to glucose-1 phosphate. Galactose metabolism may convert β-D-galactose to UDP-glucose. β-D-galactose may be converted to α-D-galactose by the enzyme, galactose mutarotase (GALM). Glycolipid transfer proteins (GLTP) may be small, soluble, proteins characterized by their ability to accelerate the intermembrane transfer of glycolipids. GLTP may accelerate glycolipid intermembrane transfer. Lectin galactoside-binding-like protein (LGALSL) may comprise a member of the galectin family of carbohydrate binding proteins. LGALSL protein may provide antimicrobial protection and may play a role in innate and adaptive immunity as well as regulate the intestinal microbiota. The biosynthesis of glycans may be determined the glycosyltransferases that assemble monosaccharide moieties into linear and branched glycan chains. When galactosyltransferase binds α-lactalbumin in a lactose synthase, it may switch its acceptor specificity from N-acetylglucosamine to glucose, which may enable lactose synthesis during milk formation.

A gene or transcript may regulate a cellular response to a hormone or a signaling factor. A gene or transcript which may regulate a cellular response may comprise a gene or a transcript selected from the group consisting of: USF, GATA-1, GATA-2, GATA-3 c-Myc:Max, ATF, USF, CREB, TATA, SREBP-1, Arnt, USF, Tal-1beta, NRF-2, FOXD3, HNF-3beta, v-Myb, FOXJ2, FOXO1, Evi-1, FOXO4, ARP-1, Staf, NF-kappaB, myogenin, AML-1a, Elk-1 Oct-1, Tax/CREB, HNF-1, AP-1, Ahr, Bachl, RP58, AREB6, NKX3A, XFD-1, deltaEF1, poly A downstream element, Pax-4, Sox-5, Sox-9, Zic3, E47, LPL, AGPAT6, CD36, SCD, GPAM, BTN1A1, ACACA, ACSL1, LPIN1, FASN, FABP3, AGPAT6, SREBP1, INSR, PRLR, EGFR, or any variant or combination thereof.

Aspects of the present disclosure may comprise a composition comprising an engineered non-mammary adult stem cell exhibiting a modified expression profile that is different than an expression profile of a corresponding non-engineered non-mammary adult stem cell, wherein the engineered non-mammary adult stem cell may be configured to be differentiated to an engineered cell exhibiting one or more characteristics of a cell of a mammary epithelial cell lineage. A mammary epithelial cell lineage may comprise a mammary epithelial luminal cell lineage. A mammary epithelial cell lineage may comprise a mammary basal cell lineage. A mammary epithelial cell lineage may comprise a precursor or progenitor cell. An engineered cell may be an engineered cell capable of lactation.

Aspects of the present disclosure may comprise a composition comprising an engineered non-mammary adult stem cell exhibiting a modified expression profile that may be different than an expression profile of a corresponding non-engineered or non-mammary adult stem cell, wherein the engineered non-mammary adult stem cell may be configured to be differentiated to an engineered cell capable of lactation. An engineered non-mammary adult stem cell may be configured to produce a milk component. An engineered non-mammary adult stem cell may be an engineered mesenchymal stem cell. A corresponding non-engineered non-mammary adult stem cell may be a corresponding non-engineered mesenchymal stem cell. A mammary cell lineage may comprise a mammary luminal cell lineage, a mammary epithelial cell lineage, a mammary alveolar cell lineage, a mammary myoepithelial cell lineage, or any variant or combination thereof.

An engineered non-mammary adult stem cell or an ancestor cell thereof may be brought in contact with a heterologous polypeptide, which heterologous polypeptide may include a gene regulating moiety. An engineered non-mammary adult stem cell may comprise a heterologous polypeptide including a gene regulating moiety. An engineered non-mammary adult stem cell may comprise a homologous polypeptide including a gene regulating moiety. An engineered non-mammary adult stem cell or an ancestor cell thereof may be brought in contact with a homologous polypeptide, which homologous polypeptide may include a gene regulating moiety.

A gene regulating moiety may comprise a polynucleotide-guided gene regulating moiety. A gene regulating moiety may comprise an endonuclease. An endonuclease may comprise a clustered regularly interspaced short palindromic repeats (CRISPR)-associated (Cas) endonuclease. A Cas endonuclease may be selected from the group consisting of: C2C1, C2C2, C2C3, Cas1, Cas1B, Cas2, Cas3, Cas4, Cas5, Cas5e, Cash, Cas6e, Cas6f, Cas7, Cas8a, Cas8a1, Cas8a2, Cas8b, Cas8c, Cas9, Cas10, Cas10d, Cas10, Cas10d, Cas11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, any modification thereof, any variant thereof, or any combination thereof.

A gene regulating moiety may be configured to regulate an expression of a target gene, a target transcript, or a target protein of the engineered non-mammary adult stem cell. An engineered non-mammary adult stem cell or an ancestor cell thereof may be brought in contact with a heterologous polynucleotide configured to form a complex with the gene regulating moiety. An engineered non-mammary stem cell may further comprise a heterologous polynucleotide configured to form a complex with a gene regulating moiety. An engineered non-mammary adult stem cell or an ancestor cell thereof may be brought in contact with a homologous polynucleotide configured to form a complex with the gene regulating moiety. An engineered non-mammary stem cell may further comprise a homologous polynucleotide configured to form a complex with a gene regulating moiety. A target gene, target transcript, or target protein may be associated with production of a milk component.

A target gene, target transcript, or target protein may be selected from a gene, a transcript, or a protein selected from the group consisting of: CIDEA, XDH, C/EBPβ, HDAC, DGAT1, DGAT2, acyl-CoA: cholesterol acyltransferase, AKT1, S14, LPL, BTN/BTN1A1, ADFP, ACACA/B, FASN, ACLY, S-acyl fatty acid synthase thioesterase, ACOT, SCD, ELOVL, FADS, ACSS, ACSL, GPD1L, GK5, DGKA, GPAM, AGPAT1, LPIN1, LPIN2, SLC27A, FABP, ABCG2, FDFT1, SC4MOL, SC5DL, IDI1, MVD, CH25H, DHCR7, PLIN2, SREBP1, MUC1, MFGE8, mTORC1, CASTOR1, GATOR1, GATOR2, RRAGA/B, RHEB, TSC2, PIK3C3, EAAT3, CAT1, 4F2hc, LAT1, ASCT2, AMPK, STK11, SIRT1, ARNTL, CLOCK, NAMPT, CRY, PER1, CSN1, CSN2, CSN3, LALBA, LTF, LYZ, LPO, TC-1, SPP1, ABL, C3, C4, Galactosyltransferase, B4GALT1, B4GALT2, LGALS1, LGALS2, LGALS3, LGALS8, LGALS9, LGALS12, LGALS13, LGALS14, LGALS16, LCT, GLUT1, FUT1, FUT2, FUT3, FUT4, FUT5, FUT6, FUT7, FUT8, FUT9, sialyltransferase, GALM, GLTP, LGALSL, N-acetylglucosaminyltransferase, glycosyltransferase, or any variant or combination thereof. A modified expression profile may comprise an up-regulated expression of a gene or a transcript that regulates production of a milk protein. A modified expression profile may comprise a down-regulated expression of a gene or a transcript that regulates production of a milk protein. A gene or the transcript regulating the production of a milk protein may be a gene or a transcript selected from the group consisting of casein and whey protein. A gene or transcript regulating the production of a milk protein may comprise a gene or a transcript selected from the group consisting of: mTORC1, CASTOR1, GATOR1, GATOR2, RRAGA/B, RHEB, TSC2, PIK3C3, EAAT3, CAT1, 4F2hc, LAT1, ASCT2, AMPK, STK11, SIRT1, ARNTL, CLOCK, NAMPT, CRY, PER1, CSN1, CSN2, CSN3, LALBA, LTF, LYZ, LPO, TC-1, SPP1, ABL, C3, C4, or any variant or combination thereof.

A modified expression profile may comprise an up-regulated expression of a gene or a transcript that regulates production of a milk carbohydrate. A modified expression profile may comprise a down-regulated expression of a gene or a transcript that regulates production of a milk carbohydrate. A gene or transcript which may regulate production of a milk carbohydrate may be selected from the group consisting of: 2′-fucosyllactose, 3-fucosyllactose, difucosyllactose, difucosyllacto-N-tetrose (DFLNT), difucosyllacto-N-hexaose, fucosyllacto-N-hexaose (FLNH), lacto-N-fucopentaose (LNFP) I, LNFP II, LNFP III, 3′-sialyllactose, 6′-sialyllactose, disialyllacto-N-hexaose (DSLNH), disialyllacto-N-tetraose (DSLNT), fucodisialyllacto-N-hexaose (FDSLNH), sialyl-lacto-N-tetraose b (LSTb), and sialyl-lacto-N-tetraose c (LSTc), lacto-N-hexaose, lacto-N-neotetraose (LNnT), lacto-N-tetrose (LNT), Lactose, or any variant or combination thereof. A gene or transcript regulating the production of a milk carbohydrate may comprise a gene or a transcript selected from the group consisting of: LALBA, Galactosyltransferase B4GALT1, Galactosyltransferase B4GALT2, LGALS1, LGALS2, LGALS3, LGALS8, LGALS9, LGALS12, LGALS13, LGALS14, LGALS16, LCT, GLUT1, FUT1, FUT2, FUT3, FUT4, FUT5, FUT6, FUT7, FUT8, FUT9, sialyltransferase, GALM, GLTP, LGALSL, N-acetylglucosaminyl transferase, glycosyltransferase, or any variant or combination thereof. A modified expression profile may comprise an up-regulated expression of a gene or a transcript that degrades lactose. A gene or transcript that degrades lactose may comprise a gene or a transcript selected from the group consisting of a Lac operon, lactase, or glycosidase.

A modified expression profile may comprise an up-regulated expression of a gene or a transcript that regulates production of a milk fat. A modified expression profile may comprise a down-regulated expression of a gene or a transcript that regulates production of a milk fat. A gene or transcript which regulates production of a milk fat may be selected from the group consisting of: palmitic acid, myristic acid, stearic acid, butyric acid, caproic acid, oleic acid, linoleic acid, α-linoleic acid, vaccenic acid, eicosapentaenoic acid, docosahexaenoic acid, calendic acid, γ-linoleic acid, eicosadienoic acid, dihomo-γ-linoleic acid, arachidonic acid, docosadienoic acid, adrenic acid, asbond acid, tetracosatetraenoic acid, tetracospentaenoic acid, hexadecatrienoic acid, stearidonic acid, eicosatrienoic acid, eicosatetraenoic acid, heneicosapentaenoic acid, docosapentaenoic acid, docosahexaenoic acid, tetracosapentaenoic acid, tetracosahexaenoic acid, rumenic acid, caprylic acid, capric acid, or any variant or combination thereof. A gene or transcript regulating the production of milk fat may comprise a gene or a transcript selected from the group consisting of: CIDEA, XDH, C/EBPβ, HDAC, DGAT1, DGAT2, acyl-CoA: cholesterol acyltransferase, AKT1, S14, LPL, BTN/BTN1A1, ADFP, ACACA/B, FASN, ACLY, S-acyl fatty acid synthase thioesterase, medium chain/OLAH, ACOT, SCD, ELOVL, FADS, ACSS, ACSL, GDP1L, GK5, DGKA, GPAM, AGPAT1, LPIN1, LPIN2, SLC27A, FABP, ABCG2, FDFT1, SC4MOL, SC5DL, IDI1, MVD, CH25H, DHCR7, PLIN2, SREBP1, MUC1, MFGE8, or any variant or combination thereof.

A gene or transcript may regulate a cellular response to a hormone or a signaling factor. A gene or transcript which regulates a cellular response may comprise a gene or a transcript selected from the group consisting of: USF, GATA-1, GATA-2, GATA-3 c-Myc:Max, ATF, USF, CREB, TATA, SREBP-1, Arnt, USF, Tal-1beta, NRF-2, FOXD3, HNF-3beta, v-Myb, FOXJ2, FOXO1, Evi-1, FOXO4, ARP-1, Staf, NF-kappaB, myogenin, AML-1a, Elk-1 Oct-1, Tax/CREB, HNF-1, AP-1, Ahr, Bachl, RP58, AREB6, NKX3A, XFD-1, deltaEF1, poly A downstream element, Pax-4, Sox-5, Sox-9, Zic3, E47, LPL, AGPAT6, CD36, SCD, GPAM, BTN1A1, ACACA, ACSL1, LPIN1, FASN, FABP3, AGPAT6, SREBP1, INSR, PRLR, EGFR, or any variant or combination thereof.

A modified expression profile may comprise (i) an up-regulated expression of a first target gene, a first target transcript, or a first target protein, and (ii) a down-regulated expression of a second target gene, a second target transcript, or a second target protein.

A modified expression profile may be indicative of a mammary cell lineage. An engineered non-mammary adult stem cell may exhibit a cellular morphology indicative of a mammary cell lineage. A mammary cell lineage may comprise a mammary epithelial cell lineage. A mammary cell lineage may comprise a mammary alveolar cell lineage. A mammary cell lineage may comprise a mammary myoepithelial cell lineage. An engineered non-mammary adult stem cell may be configured to generate a milk component in response to a hormone or a signaling factor.

An engineered non-mammary adult stem cell may comprise an exogenous polynucleotide that (1) includes an inducible promoter sequence and (2) encodes a hormone or a signaling factor. An inducible promoter sequence may be configured for inducible expression of a hormone or signaling factor. An inducible promoter sequence may be configured for activation by a reagent. An engineered non-mammary adult stem cell may have an altered response to a hormone or a signaling factor as compared to a corresponding non-engineered non-mammary adult stem cell. An engineered non-mammary adult stem cell may have an enhanced sensitivity to a hormone or a signaling factor as compared to a corresponding non-engineered non-mammary adult stem cell. An engineered non-mammary adult stem cell may have a diminished sensitivity to a hormone or a signaling factor as compared to a corresponding non-engineered non-mammary adult stem cell.

A hormone or signaling factor may be selected from the group consisting of: glucocorticoid, insulin, transferrin, apo transferrin, progesterone, prolactin, ethanolamine, estrogen, fibroblast growth factor (FGF), FGF receptor, TBX3, NRG3/ERBB4, Wnt/LEF1, insulin-like growth factor 1 (IGF-1), epidermal growth factor (EGF), hepatocyte growth factor (HGF), nuclear factor κB (NF-κB), pTHrP, non-phospho β-catenin, p-p65, RELA, CTNNB1, SMAD3, or any variant or combination thereof. A glucocorticoid may comprise beclomethasone, betamethasone, budesonide, cortisone, dexamethasone, hydrocortisone, methylprednisolone, prednisolone, prednisone, triamcinolone, or a combination thereof. An engineered non-mammary adult stem cell may be derived from a non-mammary adult stem cell isolated from a tissue or a bodily secretion of a subject.

### Methods

Aspects of the present disclosure may comprise a method for generating an engineered cell the method comprising providing a non-mammary adult stem cell or a derivative thereof; and subjecting the non-mammary adult stem cell or derivative thereof to conditions sufficient to generate an engineered cell exhibiting one or more characteristics of a cell of a mammary epithelial cell lineage.

Aspects of the present disclosure may comprise a method for generating an engineered cell may comprise isolating a cell from a tissue or a bodily secretion, contacting a cell with a growth medium and expanded a population of cells, contacting a population of expanded cells with a differentiation medium, contacting differentiated cells with a heterologous polypeptide comprising a gene regulating moiety, and introducing an exogenous nucleic acid comprising an inducible promoter sequence and encoding a hormone or a signaling factor into the population of cells.

Aspects of the present disclosure may comprise a method for generating an engineered cell comprising providing a non-mammary adult stem cell or a derivative thereof; and subjecting the non-mammary adult stem cell or derivative thereof to conditions sufficient to generate an engineered cell exhibiting one or more characteristics of a cell of a mammary epithelial cell lineage. A mammary epithelial cell lineage may comprise a mammary epithelial luminal cell lineage. A mammary epithelial cell lineage may comprise a mammary basal cell lineage. A non-mammary adult stem cell may comprise a mesenchymal stem cell. An engineered cell may be capable of lactation. An engineered cell may be capable of producing a milk component. Subjecting a non-mammary adult stem cell or derivative thereof capable of producing a milk component may be performed in vitro.

A modified expression profile may be characteristic of a mammary cell lineage. An engineered cell may comprise a cell exhibiting one or more characteristics of a cell of a mammary cell lineage. A mammary cell lineage may display a biomarker typical of the phenotype of a mammary cell linear. A modified expression profile may comprise a modified expression profile of a gene, a transcript, or a protein selected from the group consisting of: EpCAM, CD14, CD29, CD49b, CD49f, CD61, Sca1, Prominin, ALDEFLUOR, CK14, CK18, or any variant or combination thereof. A mammary cell lineage may comprise a mammary epithelial cell lineage. A mammary cell lineage may comprise a mammary alveolar cell lineage. A mammary cell lineage may comprise a mammary myoepithelial cell lineage. A mammary cell lineage may comprise any precursor or progenitor cells to such a lineage.

Aspects of the present disclosure may comprise a method for optically determining the mammary cell lineage and viability. The engineered cell described herein can be examined by optical techniques such as light microscopy, fluorescent microscopy, or electronic microscopy to determine if the engineered cells sufficiently exhibit morphology of mammary cell lineage (e.g. mammary-like cells) or terminally differentiated mammary cells. In some cases, the optical techniques can select for engineered cells that exhibit uniform morphology or characteristics of mammary-like cells or mammary cells. In some cases, the optical techniques can select for engineered cells with viability by eliminating cells that display senescence such as flat or granular morphology, presence of vacuoles. In some cases, the optical techniques such as fluorescent microscopy can determine the mammary cell lineage by imaging the biomarkers described herein exhibited by the engineered cells.

Subjecting the non-mammary adult stem cell or derivative thereof to conditions sufficient to generate an engineered cell exhibiting one or more characteristics of a cell of a mammary epithelial cell lineage may comprise contacting a non-mammary adult stem cell or derivative thereof with a heterologous polypeptide including a gene regulating moiety, wherein the gene regulating moiety may be configured to regulate an expression of a target gene, a target transcript, or a target protein of the non-mammary adult stem cell or derivative thereof, thereby effecting a modified expression profile of the target gene, target transcript, or target protein in the engineered cell as compared to a corresponding non-engineered non-mammary adult stem cell.

A target gene, the target transcript, or the target protein may be associated with production of a milk component. A target gene, the target transcript, or the target protein may be a gene or a transcript selected from or a gene or transcript associated with the group consisting of: CIDEA, XDH, C/EBPβ, HDAC, DGAT1, DGAT2, acyl-CoA: cholesterol acyltransferase, AKT1, S14, LPL, BTN/BTN1A1, ADFP, ACACA/B, FASN, ACLY, S-acyl fatty acid synthase thioesterase, ACOT, SCD, ELOVL, FADS, ACSS, ACSL, GPD1L, GK5, DGKA, GPAM, AGPAT1, LPIN1, LPIN2, SLC27A, FABP, ABCG2, FDFT1, SC4MOL, SC5DL, IDI1, MVD, CH25H, DHCR7, PLIN2, SREBP1, MUC1, MFGE8, mTORC1, CASTOR1, GATOR1, GATOR2, RRAGA/B, RHEB, TSC2, PIK3C3, EAAT3, CAT1, 4F2hc, LAT1, ASCT2, AMPK, STK11, SIRT1, ARNTL, CLOCK, NAMPT, CRY, PER1, CSN1, CSN2, CSN3, LALBA, LTF, LYZ, LPO, TC-1, SPP1, ABL, C3, C4, Galactosyltransferase, B4GALT1, B4GALT2, LGALS1, LGALS2, LGALS3, LGALS8, LGALS9, LGALS12, LGALS13, LGALS14, LGALS16, LCT, GLUT1, FUT1, FUT2, FUT3, FUT4, FUT5, FUT6, FUT7, FUT8, FUT9, sialyltransferase, GALM, GLTP, LGALSL, N-acetylglucosaminyltransferase, glycosyltransferase, or any combination or variant thereof.

Milk, particularly bovine milk, may contain more than 25 different proteins, but the whey proteins alpha-lactalbumin, beta-lactoglobulin, serum albumin, and lactoferrin, as well as the four caseins, have been identified as allergens. The identification or modulation of a suitable protein source for subjects allergic to protein components in milk may represent an important goal. Breast milk may further comprise antimicrobial and antiviral components. The antimicrobial agents may include lactoferrin, lysozyme, secretory immunoglobulin A, kappa-casein, and oligosaccharides and glycoconjugates. The antiviral agents in human milk may include mucins from the milk fat globule membrane and lactoferrin. Specific host-defense factors in milk, such as secretory immunoglobulin A and lactoferrin may resist proteolytic degradation and survive passage through the gastrointestinal tract of breastfed infants, subsequently exerting their physiologic function in the infants, and enabling an innate immunity. A gene or transcript may regulate production of a milk protein selected from the group consisting of a S1/S2 casein, β casein, κ casein, α-lactalbumin, lactoferrin, lactoperoxidase, lysozyme, haptocorrin, osteopontin, serum albumin, complement C3, complement C4, or any combination or variant thereof. A modified expression profile may comprise an up-regulated expression of a gene or a transcript that regulates production of a milk protein. The up-regulated expression of a gene or a transcript may regulate production of a milk protein. A modified expression profile may comprise a down-regulated expression of a gene or a transcript that regulates production of a milk protein. The down-regulated expression of a gene or a transcript may regulate production of a milk protein.

A casein may comprise an amphiphilic phosphoprotein present in milk. The casein fraction may be composed of alpha S1, alpha S2, beta, and κ casein, of which alpha S1casein may be a major allergen according to IgE and T cell recognition data. Bovine milk may comprise α S1/S2 casein while human milk may comprise β casein and κ casein. The casein pattern may differ depending on the stage of lactation in a lactating mammal. Casein fragments may enhance the absorption of calcium by keeping it in solution in the gut lumen. Casein derived peptides may regulate intestinal motility and promote growth of beneficial bacteria in the gut. Lactoferrin may comprise an iron binding glycoprotein present in milk belonging to the transferrin protein family. Lactoferrin may regulate iron absorption in the bowel, have antimicrobial, anti-inflammatory, and antioxidant properties. Lactoferrin may be the second most abundant protein in milk after caseins. Lactoperoxidase may be a peroxidase enzyme secreted from a mammary gland that functions as an antimicrobial agent. Lactoperoxidase may catalyze the oxidation of a number of inorganic and organic substrates by hydrogen peroxide. The lactoperoxidase system may plays a role in the innate immune system by killing bacteria in milk. Lactoperoxidase may be an effective antimicrobial agent and may be used as an antibacterial agent in reducing bacterial microflora in milk and milk products. Activation of the lactoperoxidase system by addition of hydrogen peroxide and thiocyanate may extend the shelf life of refrigerated raw milk. It may be fairly heat resistant and may be used as an indicator of over pasteurization of milk. Lysozyme may be a polypeptide which may hydrolyze the β linkages between N-acetylmuramic acid and N-acetylglucosamine, such as in the outer membrane of a bacteria. Lysozyme may be found in high quantities in milk. Human milk may comprise more lysozyme than bovine milk. Lysozyme may hydrolyze a bacterial cell wall rendering the bacteria unstable, thus acting as an antimicrobial. Lysozyme may act synergistically with IgA and lactoferrin. Haptocorrin may be expressed by mammary epithelial cells and may be present in milk. Haptocorrin is a vitamin B-12 binding protein. B-12 may be supplied exclusively to an infant through milk bound to haptocorrin. Haptocorrin may mediate vitamin B-12 absorption in breastfed infants during the neonatal period when the intrinsic factor system (the mechanism by which vitamin B-12 is absorbed in adults) may not be functioning to its full capacity. Haptocorrin may withstand proteolytic degradation and exert a bacteriostatic effect on a pathogenic bacteria strain. Haptocorrin may influence bacterial growth in the gastrointestinal tract. Osteopontin may comprise a phosphorylated acidic glycoprotein present in milk. Osteopontin may play a role in immunomodulatory activities, tissue remodeling, bone formation, as well as cell attachment, migration, proliferation, and differentiation. Osteopontin may contain RGD and non-RGD integrin binding domains, as well as a CD44-binding motif, and may exert its multiple functions by binding to its receptors on cell membranes to initiate signaling cascades. Osteopontin may be expressed more in earlier stages of lactation and may contribute to the transitional immune system of a neonate where protection against pathogens may be required. Serum albumin may be a large protein component present in milk which may make up a portion of total whey protein. Serum albumin may comprise a source of essential amino acids. Serum albumin may bind to many ligands, including fatty acids, trace elements, calcium, and other molecules. In milk, serum albumin has been associated with zinc, copper, thyroxine, fatty acids, and other small molecules. The complement system may play an important role in the defense against infection. Effector functions of complement may include opsonization, lysis of bacteria, and the attraction of phagocytes at the site of complement activation by the generation of chemotactic fragments. Complement C3 and complement C4 may be proteins in the innate immune system which may be the most commonly measured complement components. Complement C3 may be associated with triglyceride metabolism. Complement C3 and complement C4 may decrease during lactation controlling inflammatory response and offering antimicrobial protection potentials as bridge between innate and acquired immunity.

Aspects of the present disclosure may comprise a method for generating a milk nutrient. A method for generating a milk nutrient may comprise contacting a cell, such as a cell with a mammary cell lineage, with a lactogenic medium, contacting the cell with a reagent capable of regulating a cellular response to a hormone or a signaling factor, using the cell to generate an aqueous medium comprising one or more particles (for example, milk lipid globules), and isolating the milk nutrient from the aqueous medium. A food composition comprising the milk nutrient may be formulated from the isolated milk nutrient.

Aspects of the present disclosure may comprise a method for generating an engineered cell, the method comprising providing a non-mammary adult cell or a derivative thereof and subjecting the cell or derivative thereof to conditions sufficient to generate an engineered cell exhibiting one or more characteristics of a cell of a mammary epithelial cell lineage. Subjecting the cell or derivative thereof to conditions sufficient to generate an engineered cell exhibiting one or more characteristics of a cell of a mammary epithelial cell lineage may comprise contacting the non-mammary adult cell or derivative thereof with a heterologous polypeptide including a gene regulating moiety, wherein the gene regulating moiety may be configured to regulate an expression of a target gene, a target transcript, or a target protein of the non-mammary adult cell or derivative thereof, thereby effecting a modified expression profile of the target gene, target transcript, or target protein in the engineered cell as compared to a corresponding non-engineered or non-mammary adult cell.

A gene regulating moiety may comprise a polynucleotide-guided gene regulating moiety. A gene regulating moiety may comprise an endonuclease. An endonuclease may comprise a clustered regularly interspaced short palindromic repeats (CRISPR)-associated (Cas) endonuclease. A Cas endonuclease may be selected from the group consisting of: C2C1, C2C2, C2C3, Cas1, Cas1B, Cas2, Cas3, Cas4, Cas5, Cas5e, Cas6, Cas6e, Cas6f, Cas7, Cas8a, Cas8a1, Cas8a2, Cas8b, Cas8c, Cas9, Cas10, Cas10d, Cas10, Cas10d, Cas11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, any modifications thereof, variants thereof, or combinations thereof.

Subjecting the cell or derivative thereof to conditions sufficient to generate an engineered cell exhibiting one or more characteristics of a cell of a mammary epithelial cell lineage may comprise contacting the non-mammary adult cell or adult stem cell or the derivative thereof with a heterologous polynucleotide configured to bind at least a portion of the target gene or the target transcript. Subjecting the cell or derivative thereof to conditions sufficient to generate an engineered cell exhibiting one or more characteristics of a cell of a mammary epithelial cell lineage may further comprise forming a complex of the gene regulating moiety and heterologous polynucleotide. The complex may comprise a clustered regularly interspaced short palindromic repeats (CRISPR)/CRISPR-associated (Cas) protein complex.

An engineered cell may be configured to generate a milk component in response to a hormone or signaling factor. A non-mammary cell or stem cell or derivative thereof may be contacted with an exogenous polynucleotide which exogeneous polynucleotide may comprise an inducible promoter sequence and encode a hormone or a signaling factor. An inducible promoter sequence may be configured for inducible expression of a hormone or signaling factor. An inducible promoter sequence may be configured for activation by a reagent. An engineered cell may have an altered response to a hormone or a signaling factor as compared to a corresponding non-engineered or non-mammary adult cell, adult stem cell, or derivative thereof. An engineered cell may have an enhanced sensitivity to a hormone or a signaling factor as compared to a corresponding non-engineered or non-mammary adult cell, adult stem cell, or derivative thereof. An engineered cell may have a diminished sensitivity to a hormone or a signaling factor as compared to a corresponding non-engineered or non-mammary adult cell, adult stem cell, or derivative thereof.

A hormone or signaling factor may comprise a glucocorticoid, insulin, transferrin, apo transferrin, progesterone, prolactin, ethanolamine, estrogen, fibroblast growth factor (FGF), FGF receptor, T-box 3 (TBX3), NRG3/ERBB4, Wnt/LEF1, insulin-like growth factor 1 (IGF-1), epidermal growth factor (EGF), hepatocyte growth factor (HGF), nuclear factor κB (NF-κB), pTHrP, non-phospho β-catenin, p-p65, RELA, CTNNB1, SMAD3, or any variant or combination thereof. A glucocorticoid may comprise beclomethasone, betamethasone, budesonide, cortisone, dexamethasone, hydrocortisone, methylprednisolone, prednisolone, prednisone, triamcinolone, or any variant or combination thereof.

Subjecting the cell or derivative thereof to conditions sufficient to generate an engineered cell exhibiting one or more characteristics of a cell of a mammary epithelial cell lineage may comprise contacting a non-mammary adult cell, stem cell, or derivative thereof with a differentiation medium under conditions sufficient to obtain an engineered cell. Providing a non-mammary cell, adult stem cell, or a derivative thereof may comprise isolating a non-mammary cell, adult stem cell, or derivative from a tissue or a bodily secretion of a mammalian subject.

### Methods of Generating a Milk Component

Aspects of the present disclosure may comprise a method for generating a milk component, the method comprising providing a reaction vessel including a cell culture, which cell culture comprises an engineered mammary or mammary-like cell derived from a non-mammary adult stem cell; and subjecting a cell culture to conditions sufficient to produce the milk component in vitro. A cell culture may further comprise a culture medium. A milk component may comprise whey protein, casein, a lipid, an oligosaccharide, or a combination thereof. An engineered mammary or mammary-like cell may comprise an engineered milk-producing cell. A non-mammary adult stem cell may comprise a mesenchymal stem cell. An engineered mammary or mammary-like cell may comprise a genome that is different from a genome of a naturally occurring milk-producing cell. Subjecting a cell culture to conditions sufficient to produce the milk component in vitro may comprise using the non-mammary adult stem cell or a derivative thereof to generate an engineered mammary or mammary-like cell or a derivative thereof. An engineered mammary or mammary-like cell may exhibit one or more characteristics of a mammary epithelial cell. An engineered mammary or mammary-like cell may exhibit one or more characteristics of a mammary myoepithelial cell. An engineered mammary or mammary-like cell may exhibit one or more characteristics of a mammary precursor or progenitor cell, luminal cell, basal cell, or alveolar cell.

Subjecting a cell culture to conditions sufficient to produce the milk component in vitro may comprise contacting a non-mammary adult stem cell or derivative thereof with a growth medium under conditions sufficient to produce an expanded cell culture. Subjecting a cell culture to conditions sufficient to produce the milk component in vitro may comprise contacting the non-mammary adult stem cell or derivative thereof with a differentiation medium under conditions sufficient to obtain the engineered mammary or mammary-like cell.

Aspects of the present disclosure may further comprise isolating the non-mammary adult stem cell from a tissue or a bodily secretion of a subject. A tissue or bodily secretion may comprise adipose tissue, muscle tissue, cord blood, bone marrow, organ tissue, mammary tissue, extra-embryonic tissue, umbilical cord blood, tendon, periodontal ligament, synovial membrane, trabecular bone, bone marrow, nervous system, skin, periosteum, muscle, peripheral blood, breastmilk, or other body fluid, or any variant or combination thereof. Tissue or bodily secretion may comprise adipose tissue, mammary tissue, umbilical cord, umbilical cord blood, breastmilk, or any variant or combination thereof.

A subject may be a mammal. A mammal may comprise a human, a non-human primate, a horse, a cow, a dairy cow, a buffalo, a goat, a sheep, a camel, an elephant, a snow leopard, a whale, a seal, a pig, a dog, a mouse, a rat, or a rabbit.

An engineered mammary or mammary-like cell may be brought in contact with a heterologous polypeptide, which heterologous polypeptide includes a gene regulating moiety. An engineered mammary or mammary-like cell may be brought in contact with a homologous polypeptide, which homologous polypeptide includes a gene regulating moiety. A gene regulating moiety may be a polynucleotide-guided gene regulating moiety. A gene regulating moiety may be an endonuclease. An endonuclease may be a clustered regularly interspaced short palindromic repeats (CRISPR)-associated (Cas) endonuclease. A Cas endonuclease may be selected from the group consisting of: C2C1, C2C2, C2C3, Cas1, Cas1B, Cas2, Cas3, Cas4, Cas5, Cas5e, Cas6, Cas6e, Cas6f, Cas7, Cas8a, Cas8a1, Cas8a2, Cas8b, Cas8c, Cas9, Cas10, Cas10d, Cas10, Cas10d, Cas11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, a modification thereof, or any variant or combination thereof.

A cell culture may comprise a two-dimensional cell culture including the engineered mammary or mammary-like cell, a three-dimensional cell culture including the engineered mammary or mammary-like cell, or a high-density cell culture including the engineered mammary or mammary-like cell. Subjecting a cell culture to conditions sufficient to produce the milk component in vitro may comprise contacting an engineered mammary or mammary-like cell or a derivative thereof with a lactogenic medium under conditions sufficient to produce the milk component. A lactogenic medium may comprise a substance capable of activating an expression of a hormone or a signaling factor in the engineered mammary or mammary-like cell or derivative thereof. A lactogenic medium may comprise a substance capable of regulating a cellular response to a hormone or a signaling factor of the engineered milk-producing cell or derivative thereof.

Subjecting a cell culture to conditions sufficient to produce the milk component in vitro may comprise using the cell culture to generate an aqueous medium comprising one or more particles. A particle of a plurality of one or more particles may comprise a fat surrounded by a layer. A layer may comprise a milk lipid or fat. A layer may comprise a milk fat globule membrane (MFGM). A milk fat globule membrane (MFGM) may comprise a milk fat globule membrane (MFGM) protein. The one or more particles may be emulsified by the layer and dispersed in the aqueous medium. A fat may comprise a triglyceride. An aqueous medium may comprise a carbohydrate. A carbohydrate may comprise a monosaccharide, a disaccharide, an oligosaccharide, or any variant or combination thereof.

An oligosaccharide may comprise from 3 to 20 saccharide units. An oligosaccharide may comprise, at least 1, 2, 3, 4, 5, 6, 7, 8, 9, 10, 15, 20, 25, 30, 35, 40, 45, 50, 60, 70, 80, 90, 100, or more than 100 saccharide units. An oligosaccharide may comprise a terminal lactose moiety. A carbohydrate may comprise a plurality of oligosaccharides comprising an oligosaccharide. A milk component may not comprise lactose. A milk component may comprise lactose. A milk component may comprise a fat, a protein, a carbohydrate, or any variant or combination thereof. A protein may comprise casein, whey protein, alpha-lactalbumin, lactoferrin, immunoglobulin IgA, lysozyme, serum albumin, or any variant or combination thereof.

A milk component may be isolated from the cell culture. A method of generating a milk component may further comprise isolating the milk component from the cell culture. A method of generating a milk component may result in a sterile composition comprising the milk component. A sterile component may be substantially free of whole cells. A food composition comprising a milk component, wherein the milk component may be produced by methods of the present disclosure. A food composition or food product may comprise a milk component wherein the milk component may be produced by one or more engineered mammary or mammary-like cells derived from a non-mammary adult stem cell. A milk component may be produced by the one or more engineered mammary or mammary-like cells in a culture medium.

A food composition may comprise a dairy composition selected from the group consisting of: milk, yogurt, cheese, cream, or butter. Milk may be selected from colostrum, mature milk, fore milk, hind milk, or transition milk. Milk may be formulated for a subject of an age. Milk may be formulated for a child of an age three-years or younger. Milk may be formulated for an infant of twelve-months old or younger. Milk may be formulated for a 1, 2, 3, 4, 5, 6, 7, 8, 9, 10, 15, 20, 25, 30, 35, 40, 45, 50, 60, 70, 80, 90, 100-year-old, or older than 100-year-old subject. Milk may be formulated for a less than one-year old subject. A subject may be a neonate, an infant, a baby, a toddler, a juvenile, an adolescent, a young adult, an adult, or an elderly subject.

A food composition may be sterile. A food composition may not be sterile. A food composition may be lactose-free. A food composition may comprise lactose. A food composition may further comprise a mineral. A food composition may further comprise a vitamin. A food composition may further comprise an amino acid. A food composition may further comprise one or more probiotic bacteria. The one or more probiotic bacteria may comprise one or more strains selected from the group consisting of: Bifidobacterium, Lactobacillus, Clostridium, Ralstonia, Staphylococcus, Streptococcus, and any variant or combination thereof. The one or more probiotic bacteria may comprise one or more strains selected from the group consisting of: Pseudomonas, Staphylococcus, Streptococcus, Elizabethkingia, Variovorax, Bifidobacterium, Flavobacterium, Lactobacillus, Stenotrophomonas, Brevundimonas, Chryseobacterium, Enterobacter, or any variant or combination thereof. A food composition may further comprise an antibody. An antibody may comprise Immunoglobulin IgA. A food composition may further comprise lactoferrin.

A milk component may comprise a fat, a protein, a carbohydrate, or any variant or combination thereof. A protein may comprise casein, whey protein, alpha-lactalbumin, lactoferrin, immunoglobulin IgA, lysozyme, serum albumin, or any variant or combination thereof. A carbohydrate may comprise a monosaccharide, a disaccharide, an oligosaccharide, or any variant or combination thereof. An oligosaccharide may comprise 1, 2, 3, 4, 5, 6, 7, 8, 9, 10, 15, 20, 25, 30, 35, 40, 45, 50, 60, 70, 80, 90, 100, or more than 100, or a range between any two of the foregoing, saccharide units. An oligosaccharide may comprise a terminal lactose moiety. A carbohydrate may comprise one or a plurality of oligosaccharides. A carbohydrate may not comprise lactose. A carbohydrate may comprise lactose. A fat may comprise a triglyceride. A fat may comprise a fat or lipid other than a triglyceride.

A milk component may comprise, by weight, from about 0.1% to about 20% protein. A milk component may comprise, by weight, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% 15%, or 20% of at least one protein. A milk component may comprise, by weight, at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% 15%, or 20% of at least one protein. A milk component may comprise, by weight, about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% 15%, or 20%, or a range between any two foregoing values, of at least one protein. A milk component may comprise, by weight, less than about 0.1% of at least one protein. A milk component may comprise, by weight, at least about 1%, 2%, 3%, 4%, 5%, 6%, 7%, 8%, 9%, 10%, 15%, 20%, 25%, or 30% of at least one carbohydrate. A milk component may comprise, by weight, at most about 1%, 2%, 3%, 4%, 5%, 6%, 7%, 8%, 9%, 10%, 15%, 20%, 25%, or 30% of at least one carbohydrate. A milk component may comprise, by weight, about 1%, 2%, 3%, 4%, 5%, 6%, 7%, 8%, 9%, 10%, 15%, 20%, 25%, or 30%, or a range between any two foregoing values, of at least one carbohydrate. A milk component may comprise by weight at most about 30% carbohydrate. A milk component may comprise, by weight, at least about 1%, 2%, 3%, 4%, 5%, 6%, 7%, 8%, 9%, 10%, 15%, 20%, 25%, or 30% of lactose. A milk component may comprise, by weight, at most about 1%, 2%, 3%, 4%, 5%, 6%, 7%, 8%, 9%, 10%, 15%, 20%, 25%, or 30% of lactose. A milk component may comprise, by weight, about 1%, 2%, 3%, 4%, 5%, 6%, 7%, 8%, 9%, 10%, 15%, 20%, 25%, or 30%, or a range between any two foregoing values, of lactose. A milk component may comprise by weight at most about 30% lactose. A milk component may comprise, by weight, at least about 1%, 2%, 3%, 4%, 5%, 6%, 7%, 8%, 9%, 10%, 15%, 20%, 25%, 30%, 35%, 40%, 45%, 50%, 55%, or 60% of at least one fat. A milk component may comprise, by weight, at most about 1%, 2%, 3%, 4%, 5%, 6%, 7%, 8%, 9%, 10%, 15%, 20%, 25%, 30%, 35%, 40%, 45%, 50%, 55%, or 60% of at least one fat. A milk component may comprise, by weight, about 1%, 2%, 3%, 4%, 5%, 6%, 7%, 8%, 9%, 10%, 15%, 20%, 25%, 30%, 35%, 40%, 45%, 50%, 55%, or 60%, or a range between any two foregoing values, of at least one fat. A milk component may comprise by weight at most about 60% fat.

A milk component may comprise, by weight, from at least about 3% to 4%, 3% to 5%, 3% to 6%, 3% to 7%, 3% to 8%, 3% to more than 8%, 4% to 5%, 4% to 6%, 4% to 7%, 4% to 8%, 4% to more than 8%, 5% to 6%, 5% to 7%, 5% to 8%, 5% to more than 8%, 6% to 7%, 6% to 8%, 6% to more than 8%, 7% to 8%, 7% to more than 8%, 8% to more than 8% fat. A milk component may comprise, by weight, less than about 3% fat. A milk component may comprise, by weight, from at least about 1% to 2%, 1% to 3%, 1% to 4%, 1% to 5%, 1% to 6%, 1% to 7%, 1% to more than 7%, 2% to 3%, 2% to 4%, 2% to 5%, 2% to 6%, 2% to 7%, 2% to more than 7%, 3% to 4%, 3% to 5%, 3% to 6%, 3% to 7%, 3% to more than 7%, 4% to 5%, 4% to 6%, 4% to 7%, 4% to more than 7%, 5% to 6%, 5% to 7%, 5% to more than 7%, 6% to 7%, 6% to more than 7%, or 7% to more than 7% protein. A milk component may comprise, by weight, less than about 1% protein. A milk component may comprise, by weight, from at least about 1% to 2%, 1% to 3%, 1% to 4%, 1% to 5%, 1% to 6%, 1% to 7%, 1% to more than 7%, 2% to 3%, 2% to 4%, 2% to 5%, 2% to 6%, 2% to 7%, 2% to more than 7%, 3% to 4%, 3% to 5%, 3% to 6%, 3% to 7%, 3% to more than 7%, 4% to 5%, 4% to 6%, 4% to 7%, 4% to more than 7%, 5% to 6%, 5% to 7%, 5% to more than 7%, 6% to 7%, 6% to more than 7%, or 7% to more than 7% carbohydrate. A milk component may comprise, by weight, less than about 1% carbohydrate.

### Kits

Aspects of the present disclosure may comprise kits. A kit may comprise a CRISPR kit. A CRISPR kit may comprise a heterologous polypeptide, a (polynucleotide-guided) gene regulating moiety, an endonuclease wherein the endonuclease may comprise a clustered regularly interspaced short palindromic repeats (CRISPR)-associated (Cas) endonuclease, a heterologous polynucleotide, and a complex. A kit may comprise a culture medium or a plurality of culture media.

### Computer Systems

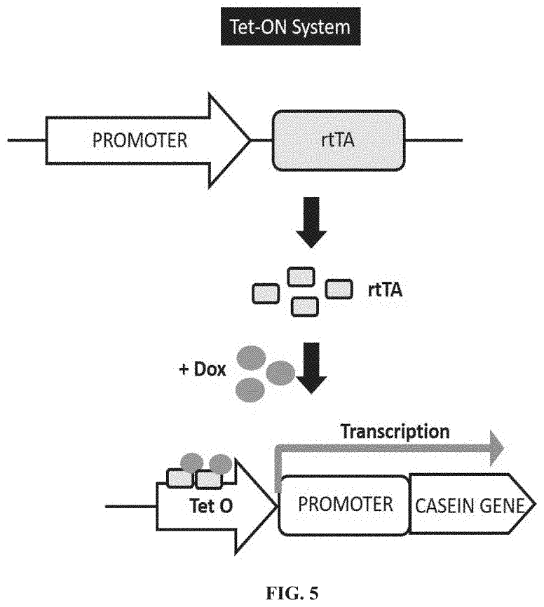

The present disclosure provides computer systems that are programmed to implement methods of the disclosure. FIG. 5 shows a computer system 101 that is programmed or otherwise configured to perform the methods described herein. The computer system 101 can regulate various aspects of the present disclosure, such as, for example, determining the ratio of media supplied to a culture in a bioreactor. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user (e.g., a cellular network). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine-readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture,” e.g., in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140, for example, determining a ratio of media supplied to a culture or the flow rate of media entering in a bioreactor. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105. The algorithm can, for example, determine the ratio of media supplied to a culture or the flow rate of media entering in a bioreactor.

### EXAMPLES

### Example 1: Isolation of Stem Cells

Examples 1a-1d illustrate isolation of non-mammary adult stem cells in general, and mesenchymal stem cells in particular, from various sources.

### Example 1a: Isolation of Mesenchymal Stem Cells from Adipose Tissue

This example illustrates isolation of mesenchymal stem cells from adipose tissue of different mammals.

Fresh adipose tissue is obtained from slaughterhouses or liposuction (e.g., in case of humans, after informed consent), kept in phosphate buffer saline supplemented with antibiotics. Enzymatic digestion of the adipose tissue is performed followed by centrifugation to isolate mesenchymal stem cells. The isolated mesenchymal stem cells are transferred to cell culture flasks and grown under standard growth conditions, e.g., 37° C., 5% CO₂. The initial culture medium include DMEM-F12, RPMI, and Alpha-MEM medium (supplemented with 15% fetal bovine serum), and 1% antibiotics. The culture medium is subsequently replaced with 10% FBS (fetal bovine serum)-supplemented media after the first passage. For example, Ahmad and Shakoori (2013) (J. Stem Cell Regen Med. 9(2): 29-36), which is incorporated herein by reference in its entirety for all purposes, describes certain variation(s) of the method(s) described herein in this example.

### Example 1b: Isolation of Mesenchymal Stem Cells from Umbilical Cord and Cord Blood

This example illustrates isolation of mesenchymal stem cells from umbilical cord or/and cord blood of mammal(s) with healthy pregnancy, for instance, at the end of the gestation period.

For humans, umbilical cord or cord blood of healthy pregnancy/pregnancies are obtained at the end of the gestation period from hospital(s) after obtaining maternal consent. The umbilical cord is collected in phosphate buffer saline supplemented with antibiotics; and the cord blood is collected in tubes with anticoagulant. The collected umbilical cord is cut into 5 cm² pieces under sterile conditions, followed by removal of blood vessels. The segments of umbilical cord are then transferred in cell culture plates containing DMEM-F12, RPMI, and Alpha-MEM (supplemented with 10% fetal bovine serum), and 1% antibiotics for seven days. Explants are removed and media is changed after seven days with fresh growth medium. Mesenchymal stem cells from cord blood are isolated by density gradient centrifugation by adding Ficoll-Paque solution, cells from ficoll-media interphase are collected and added to the aforementioned complete media. Growth medium is supplemented with dexamethasone to reduce adherence of monocytes. Non-adherent cells are removed after seven days of isolation by changing the culture medium with fresh complete growth medium. For example, Tropel et al. (2004) (Exp Cell Res. 295(2): 395-406), Van Pham et al. (2016) (Cell Tissue Bank. 17(2): 289-302), and Hassan et al. (2017) (Int J Stem Cell. 10(2): 184-192), each of which is incorporated herein by reference in their entireties for all purposes, alone or in combination(s), describe certain variation(s) of the method(s) described herein in this example.

### Example 1c: Isolation of Mesenchymal Stem Cells from Mammary Tissue

This example illustrates isolation of mesenchymal stem cells from healthy mammary tissue of any lactating mammal, including human or any other animal(s) such as described herein in this disclosure.

Fresh mammary tissue is obtained from slaughterhouses and is washed with phosphate buffer saline (PBS). The obtained mammary tissue is cut into 1 mm³ pieces and kept in a culture medium under standard culture conditions. Alternatively, the obtained mammary tissue is subjected to enzymatic digestion followed by density gradient centrifugation. The cells are then washed thrice with phosphate buffer saline and seeded in culture plates in DMEM-F12, RPMI, and Alpha-MEM medium under standard culture conditions. After preliminary plating, the media is changed every two or three days. Adherent fibroblast-like cells appear after ten days of culturing and are subsequently trypsinized into another flask for expansion. The cells of passage 3 are then used for differentiation into mature breast luminal or/and epithelial cells. For example, Huynh et al. (1991) (Exp Cell Res. 197(2): 191-99), Gibson et al. (1991) (In Vitro Cell Dev Biol Anim. 27(7): 585-594), Blatchford et al. (1999) (Animal Cell Technology: Basic & Applied Aspects. Springer, Dordrecht. pp. 141-145); and Zhang et al. (2013) (Oncol. Lett. 6(6): 1577-1582), each of which is incorporated herein by reference in their entireties for all purposes, alone or in combination(s), describe certain variation(s) of the method(s) described herein in this example.

### Example 1d: Isolation of Mesenchymal Stem Cells from Milk

This example illustrates isolation of mesenchymal stem cells from milk collected under aseptic conditions from human or any other mammal(s) such as described herein in this disclosure.

An equal volume of phosphate buffer saline is added to diluted milk, followed by centrifugation for 20 min. The cell pellet is washed thrice with phosphate buffer saline and cells are seeded in cell culture flasks in DMEM-F12, RPMI, and Alpha-MEM medium supplemented with 10% fetal bovine serum and 1% antibiotics under standard culture conditions. For example, Hassiotou et al. (2012), Stem Cells. 30(10): 2164-2174, which is incorporated herein by reference in its entirety for all purposes, describes certain variation(s) of the method(s) described herein in this example.

### Example 2: Differentiation of Stem Cells Using 2D and 3D Culture Systems

The cells isolated from any of the above-referenced sources can be differentiated into mammary epithelial and mammary luminal cells in 2D and 3D culture systems. For 2D culture, the isolated stem cells obtained were initially seeded in culture plates in growth media supplemented with 10 ng/ml epithelial growth factor and 5 μg/ml insulin. At confluence, cells were fed with growth medium supplemented with 2% fetal bovine serum, 1% penicillin-streptomycin (100 U/ml penicillin, 100 ug/ml streptomycin), and 5 μg/ml insulin for 48 h. To induce differentiation, the cells were fed with complete growth medium containing 5 μg/ml insulin, 1 μg/ml hydrocortisone, 0.65 ng/ml triiodothyronine, 100 nM dexamethasone, and 1 μg/ml prolactin. After 24 h serum is removed from the complete induction medium. For 3D culture, the isolated stem cells were trypsinized and cultured in Matrigel, hyaluronic acid, or ultra-low attachment surface culture plates for six days and induced to differentiate and lactate by adding growth media supplemented with long/ml epithelial growth factor and 5 μg/ml insulin. At confluence, cells were fed with growth medium supplemented with 2% fetal bovine serum, 1% penicillin-streptomycin (100 U/ml penicillin, 100 ug/ml streptomycin), and 5 μg/ml insulin for 48 h. To induce differentiation, the cells were fed with complete growth medium containing 5 μg/ml insulin, 1 μg/ml hydrocortisone, 0.65 ng/ml triiodothyronine, 100 nM dexamethasone, and 1 μs/ml prolactin. After 24 h, serum is removed from the complete induction medium.

See, for example, Huynh et al. 1991. Exp Cell Res. 197(2): 191-199; Gibson et al. 1991, In Vitro Cell Dev Biol Anim. 27(7): 585-594; Blatchford et al. 1999; Animal Cell Technology: Basic & Applied Aspects, Springer, Dordrecht. 141-145; Williams et al. 2009, Breast Cancer Res, 11(3): 26-43; and Arevalo et al. 2015, Am J Physiol Cell Physiol. 310(5): C348-C356; each of which is incorporated herein by reference in their entireties for all purposes.

### Example 3: Use of CRISPR-Cas Endonuclease to Engineer Mammary Cells

This example illustrates methods for CRISPR-Cas engineering of mammary or mammary-like cells as described in the present disclosure.

To convert Cas9 from a DNA scissor into a gene activator, it is necessary to disrupt its nuclease activity. Cas9's two nuclease domains, the RuvC and HNH domains, are conserved among several types of nucleases, and each is responsible for cutting one strand of DNA upon binding. One type of effector that is fused to dCas9 is a transcriptional activator. There are different forms of these dCas9-activator fusions. In eukaryotic cells, the first generation of dCas9 activators consisted of dCas9 fused to the activation domain of p65 or a VP64 activator, an engineered tetramer of the herpes simplex VP16 transcriptional activator domain. There have been several attempts to improve the direct fusion design for the second generation of CRISPRa.

One can use the approach, termed the synergistic activation mediator (SAM) system, described by Konermann et al., entitled “Genome-scale transcriptional activation by an engineered CRISPR-Cas9 complex,” Nature (2015), 517(7536):583-588, which is incorporated herein by reference in its entirety for all purposes. The SAM system employs multiple transcriptional activators to create a synergistic effect. This tool makes use of the first-generation version of dCas9-VP64, with additional features engineered into the sgRNA to enhance activator recruitment. This sgRNA contains two copies of an RNA hairpin from the MS2 bacteriophage, which interacts with the RNA-binding protein (RBP) MCP (MS2 coat protein).

The β-casein CRISPR Activation Plasmid is a synergistic activation mediator (SAM) transcription activation system designed to specifically upregulate gene expression. This Plasmid consists of three plasmids at a 1:1:1 mass ratio: a plasmid encoding the deactivated Cas9 (dCas9) nuclease (D10A and N863A) fused to the transactivation domain VP64, and a blasticidin resistance gene; a plasmid encoding the MS2-p65-HSF1 fusion protein, and a hygromycin resistance gene; a plasmid encoding a target-specific 20 nucleotide (nt) guide RNA fused to two MS2 RNA aptamers, and a puromycin resistance gene. The resulting SAM complex binds to a site-specific region approximately 200-250 nt upstream of the transcriptional start site and provides robust recruitment of transcription factors for highly efficient gene activation.

β-casein CRISPR Activation Plasmid is transfected into mammary cell lines transiently by adjusting the cell and reagent amounts proportionately for wells or dishes of different sizes. The transfection reagent concentration, 0.5-5 of each plasmid, is introduced into each well with the transfection reagent. Following transfection, stable activated clones are selected via antibiotic selection. The gene activation efficiency is assayed by WB, IF or IHC using β-casein antibody.

### Example 4: Method of Making Mammary-Like Cells

This example illustrates methods for making mammary or mammary-like cells as described in the present disclosure.

Mammalian cells are brought to induced pluripotency by reprogramming with viral vectors encoding for Oct4, Sox2, Klf4, and c-Myc. The Resultant Reprogrammed Cells are then Cultured in Mammocult media (available from Stem Cell Technologies), or mammary cell enrichment media (DMEM, 3% FBS, estrogen, progesterone, heparin, hydrocortisone, insulin, EGF) to make them mammary like, from which expression of select milk components can be induced. Alternatively, epigenetic remodeling are performed using remodeling systems such as CRISPR/Cas9, to activate select genes of interest, such as casein, α-lactalbumin to be constitutively on, to allow for the expression of their respective proteins.

### Example 5: Method of Culturing Cells

This example illustrates methods for cell culturing as described in the present disclosure.

Completed growth media includes high glucose DMEM/F12, 10% FBS, 1% NEAA, 1% pen/strep, 1% ITS-X, 1% L-Glu, long/ml EGF, and 5 μg/ml hydrocortisone. Completed lactation media includes high glucose DMEM/F12, 1% NEAA, 1% pen/strep, 1% ITS-X, 1% L-Glu, 10 ng/ml EGF, 5 μg/ml hydrocortisone, and 1 μg/ml prolactin (5 ug/ml in Hyunh 1991). Cells are seeded at a density of 20,000 cells/cm² onto collagen coated flasks in completed growth media and left to adhere and expand for 48 hours in completed growth media, after which the media is switched out for completed lactation media. Upon exposure to the lactation media, the cells start to differentiate and stop growing. Within about a week, the cells start secreting lactation product(s) such as milk lipids, lactose, casein and whey into the media. A desired concentration of the lactation media can be achieved by concentration or dilution by ultrafiltration. A desired salt balance of the lactation media can be achieved by dialysis, for example, to remove unwanted metabolic products from the media. Hormones and other growth factors used can be selectively extracted by resin purification, for example the use of nickel resins to remove His-tagged growth factors, to further reduce the levels of contaminants in the lactated product.

### Example 6: Cell-Produced Nutrient Compositions

This example provides a non-limiting example of compositions obtained from in vitro culturing of mammary epithelial cells or mammary-like cells. The compositions (e.g., liquid compositions) illustrated hereinbelow in this example are substantially free of immunoglobulin(s), substantially free of whole cells, or substantially free of bacterial microbe(s).

In a non-limiting example, a composition (e.g., liquid composition) obtained from bovine mammary epithelial cells or bovine mammary-like cells can include, by weight, about 40% to about 90% water, about 0.1% to about 10% lactose, about 0.1% to about 10% protein, at most about 60% fat, and about 0.1% to about 3% mineral(s). A composition (such as a liquid composition) of the present disclosure, for example, obtained from an in vitro culture of bovine mammary cells as described herein can contain at least one ingredient or component as listed in Table 1.

TABLE 1
Non-limiting example compositions obtainable from bovine mammary
cells, optionally supplemented with additional components.

| | Bovine | Bovine |
|---|---|---|
| | Composition A | Composition B |
| Total Solids (%) | 12.5 | 15.9 |
| Fat (%) | 3.9 | 7.8 |
| Protein (%) | 3.3 | 6.6 |
| Casein (%) | 2.6 | 5.2 |
| Whey (%) | 0.7 | 1.4 |
| Fat/Protein | 1.18 | 1.18 |
| Fat/Casein | 1.50 | 1.50 |
| Ash (%) | 0.7 | |
| Total Calcium (mM) | 32 | 50 |
| Ionic Calcium (mM) | 3 | 6 |
| Soluble Calcium (mM) | 7 | 0 |
| Colloidal Calcium (mM) | 22 | 44 |
| Total Phosphorous (mM) | 30 | 38 |
| Ionic Phosphate (mM) | 5 | 0 |
| Soluble Phosphorous (mM) | 6 | 0 |
| Colloidal Phosphorous (mM) | 19 | 38 |
| Lactose % | 4.6 | 1.5 |
| | mg/ml | mg/ml |
| a-lactalbumin | 1.2 | 0 |
| b-Lactoglobulin | 3 | 0 |
| a-s1-casein | 11.6 | 23.2 |
| a-s2-casein | 3 | 6 |
| g-casein | 1.6 | 3.2 |
| b-casein | 9.6 | 19.2 |
| k-casein | 3.6 | 7.2 |
| Immunoglobulins | 0.6 | 0 |
| Lactoferrin | 0.3 | 0 |
| Lysozyme | Trace | 0 |
| Serum Albumin | 0.4 | 0 |
| Other | 0.6 | 0 |

In another non-limiting example, a composition (e.g., liquid composition) obtained from human mammary cells or human mammary-like cells can include, by weight, about 40% to about 90% water, about 0.1% to about 30% lactose, about 0.1% to about 8% protein, about 0.1% to about 10% fat, and about 0.1% to about 3% mineral(s). A composition (such as a liquid composition) of the present disclosure, for example, obtained from an in vitro culture of human mammary cells as described herein can contain at least one ingredient or component as listed in Table 2.

TABLE 2
Non-limiting example compositions obtainable from human mammary
cells, optionally supplemented with additional components.

| | Human |
|---|---|
| | Composition C |
| Protein (g/100 g) | 1.20 |
| Casein (g/100 ml) | 0.44 |
| a-s1-casein (g/100 ml) | |
| a-s2-casein (g/100 ml) | |
| g-casein (g/100 ml) | |
| b-casein (g/100 ml) | 0.41 |
| k-casein (g/100 ml) | 0.03 |
| Whey (g/100 ml) | 0.77 |
| a-lactalbumin (g/100 ml) | 0.33 |
| b-Lactoglobulin (g/100 ml) | 0 |
| Immunoglobulins (g/100 ml) | 0.11 |
| Lactoferrin (g/100 ml) | 0.218 |
| Lysozyme (g/100 ml) | 0.055 |
| Serum Albumin (g/100 ml) | 0.055 |
| Nonprotein Nitrogen Containing Molecules | |
| Free Amino Acids | |
| Creatine | |
| Carnitine | |
| Nucleotides (e.g., cytidine-monophosphate (CMP), | |
| uridine-monophosphate (UMP), adenosine- | |
| monophosphate (AMP), guanosine-monophosphate | |
| (GMP), inosine-monophosphate (IMP), and cytidine | |
| 5’ diphosphate (CDP)) | |
| Polyamines (e.g., putrescine, spermidine, spermine) | |
| Fat (%) | 3.7 |
| Saturated Fatty Acids (g/100 ml) | 1.5 |
| Trans Fatty Acids (g/100 ml) | 0.032 |
| Mono-unsaturated Fatty Acids (g/100 ml) | 1.47 |
| Poly-unsaturated Fatty Acids (g/100 ml) | 0.466 |
| Omega 3 Fatty Acids (g/100 ml) | 0.061 |
| n-3 LC PUFA | 0.018 |
| n-3 PUFA | 0.042 |
| Omega 6 Fatty Acids (g/100 ml) | 0.401 |
| n-6 LC PUFA | 0.045 |
| n-6 PUFA | 0.356 |
| n-3/n-6 ratio | 0.152 |
| 18:2n-6 (Linoleic Acid) (g/100 ml) | 0.358 |
| 20:4n-6 (Arachidonic Acid) (g/100 ml) | 0.016 |
| 18:3n-3 (a-Linolenic Acid) (g/100 ml) | 0.043 |
| 20:5n-3 (EPA) (g/100 ml) | 0.004 |
| 22:5n-3 (DPA) (g/100 ml) | 0.005 |
| 22:6n-3 (DHA) (g/100 ml) | 0.008 |
| Capric acid C10:0 (g/100 ml) | 0.049 |
| Lauric acid C12:0 (g/100 ml) | 0.191 |
| Myristic acid C14:0 (g/100 ml) | 0.213 |
| Palmitic Acid C16:0 (g/100 ml) | 0.78 |
| Palitoleic acid C16: ln-7 (g/100 ml) | 0.097 |
| Margaric Acid C17:0 (g/100 ml) | 0.014 |
| Stearic acid C18:0 (g/100 ml) | 0.255 |
| Oleic acid C18: 1n-9c (g/100 ml) | 1.294 |
| Vaccenic acid C18: 1n-7t (g/100 ml) | 0.049 |
| Gondoic (11-Eicosenoic) acid C20: 1n-9 (g/100 ml) | 0.014 |
| Dihomo-1-linolenic (cis-8,11,14-Eicosatreienoic | 0.015 |
| acid) C20:3n-6 (g/100 ml) | |
| Total Fatty Acids (g/100 ml) | 3.44 |
| Carbohydrate (%) | 7.28 |
| Lactose (g/100 ml) | 7.24 |
| Glucose (g/100 ml) | 0.024 |
| Galactose (g/100 ml) | 0.013 |
| Oligosaccharides (g/100 ml) | 2.4 |
| Vitamins | |
| Vitamin A (mcg/100 ml) | 60 |
| Vitamin D (mcg/100 ml) | 0.1 |
| Vitamin E (mg/100 ml) | 0.25 |
| Vitamin K (mcg/100 ml) | 0.2 |
| Vitamin C (mg/100 ml) | 5.5 |
| Thiamin (mcg/100 ml) | 20 |
| Riboflavin (mcg/100 ml) | 35 |
| Niacin (mg/100 ml) | 0.1 |
| Vitamin B6 (mcg/100 ml) | 15 |
| Folic Acid (mcg/100 ml) | 8.5 |
| Pantothenic Acid (mcg/100 ml) | 0.26 |
| Vitamin B12 (mcg/100 ml) | 0.2 |
| Biotin (mcg/100 ml) | 0.15 |
| Inositol (mg/100 kcal) | 20 |
| Choline (mg/100 kcal) | 13 |
| Ash (%) | 0.2 |
| Calcium (mg/100 ml) | 31 |
| Phosphorous (mg/100 ml) | 14 |
| Magnesium (mg/100 ml) | 10 |
| Iron (mg/100 ml) | 0.03 |
| Iodine (mcg/100 ml) | 17.00 |
| Selenium (mcg/100 ml) | 2.000 |
| Copper (mg/100 ml) | 0.025 |
| Zinc (mg/100 ml) | 2.19 |
| Manganese (mcg/100 ml) | 0.06 |
| Sodium (mg/100 ml) | 18 |
| Potassium (mg/100 ml) | 52.5 |
| Chloride (mg/100 ml) | 42 |

### Example 6: Using CRISPR/Cas to Knock in an Exogenous Nucleic Acid

This example illustrates a method for CRISPR/Cas9 engineering of mammary or mammary-like cells as described in the present disclosure for knocking in an exogenous nucleic acid. In some cases, the exogenous nucleic acid can be a regulatory region such as enhancer, promoter, silencer, insulator, or operator. In some cases, the exogenous nucleic acid comprises a promoter, where the promoter can be an inducible promoter (e.g. the inducible promoter system described in FIG. 4). In some cases, the exogenous nucleic acid being knocked in the mammary or mammary-like cells by the CRISPR/Cas described herein can encode any one of the gene described herein. In some cases, the exogenous nucleic acid can encode an exogenous gene. For example, the exogenous nucleic acid can encode any one of the following exogenous gene: glucocorticoid, insulin, transferrin, apo transferrin, progesterone, prolactin, ethanolamine, estrogen, fibroblast growth factor (FGF), FGF receptor, TBX3, NRG3/ERBB4, Wnt/LEF1, insulin-like growth factor 1 (IGF-1), epidermal growth factor (EGF), hepatocyte growth factor (HGF), nuclear factor κB (NF-κB), pTHrP, non-phospho β-catenin, p-p65, RELA, CTNNB1, SMAD3, beclomethasone, betamethasone, budesonide, cortisone, dexamethasone, hydrocortisone, methylprednisolone, prednisolone, prednisone, or triamcinolone.

At least one vector encoding the components of the CRISPR/Cas9 (e.g. gRNA, exogenous nucleic acid to be knocked in, or Cas9) is introduced into the mammary or mammary-like cell. Cas9 performs a site-directed digestion to cut the chromosome of the mammary or mammary-like cell at a precise location targeted by the gRNA. The break in the chromosome is subsequently repaired by inserting the exogenous nucleic acid into the chromosome, thus knocking in the exogenous nucleic acid comprising the exogenous regulatory region or the exogenous gene.

The gene expression stemmed from the knocked in exogenous regulatory region or the knocked in exogenous gene can be measured by nucleic acid-based detection techniques to detect mRNA of the gene that is regulated by the knocked-in exogenous regulatory region or mRNA of the knocked in exogenous gene. In an example, nucleic acid-based detection technique can include quantitative polymerase chain reaction (qPCR), gel electrophoresis, immunochemistry, in situ hybridization such as fluorescent in situ hybridization (FISH), cytochemistry, or next generation sequencing. In some embodiments, the nucleic acid-based detection technique can involve TaqMan™ qPCR, which involves a nucleic acid amplification reaction with a specific primer pair, and hybridization of the amplified nucleic acids with a hydrolysable probe specific to a target nucleic acid. In some instances, the nucleic acid-based detection technique can include hybridization and/or amplification assays such as Southern or Northern analyses, polymerase chain reaction analyses, and probe arrays. In some embodiments, the gene expression stemmed from the knocked in exogenous regulatory region or the knocked in exogenous gene can be measured by polypeptide detection techniques to detect polypeptide encoded by the gene that is regulated by the knocked-in exogenous regulatory region or polypeptide encoded by the knocked in exogenous gene. In an example, polypeptide detection technique can include electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), enzyme-linked immunosorbent assays (ELISAs), immunofluorescent assays, or Western blotting.

In some embodiments, the CRISPR/Cas9 engineered mammary or mammary-like cell can be screened for the expression level of a knocked in exogenous gene, where the mammary or mammary-like cell can then be cloned and proliferated. For example, mammary or mammary-like cells comprising a knocked in exogenous nucleic acid encoding insulin can be screened for the expression of insulin. The mammary or mammary-like cells expressing sufficient or increased insulin as determined by the nucleic acid detection or the polypeptide detection techniques described herein can then be cloned, expanded, and stored until these cells are used to produce the composition described here herein.

### Example 7. Using CRISPR/Cas9 or Cas 12 to Knock Out an Endogenous Gene

This example illustrates a method for CRISPR/Cas such as Cas9 or Cas12 knocking out an endogenous gene in mammary or mammary-like cells. In some embodiments, the endogenous gene can be any one of the gene described herein. In some embodiments, the endogenous gene can be any one of the casein described herein. In some embodiments, the endogenous gene can be an allergen described herein. In some cases, the endogenous gene can be a gene (e.g. LALBA) that is involved in lactose synthesis.

At least one vector encoding the components of the CRISPR/Cas9 or Cas12 (e.g. gRNA, Cas9, or Cas12) is introduced into the mammary or mammary-like cell. Cas9 or Cas12 performs a site-directed digestion to cut the chromosome of the mammary or mammary-like cell at a precise location targeted by the gRNA. The break in the chromosome knocks out the expression of the endogenous gene.

The gene expression of the knocked out endogenous gene can be measured by any of the nucleic acid-based detection techniques described herein to detect mRNA of the knocked out endogenous gene or by polypeptide detection techniques to detect polypeptide encoded by the knocked out endogenous gene.

In some embodiments, the CRISPR/Cas9 engineered mammary or mammary-like cells can be screened for the expression level of the knocked out endogenous gene, where the mammary or mammary-like cells can then be cloned and proliferated. For example, mammary or mammary-like cells comprising a knocked out endogenous gene of an allergen can be screened for the expression of the allergen. The mammary or mammary-like cells exhibiting suppressed or absent expression of the allergen as determined by the nucleic acid detection or the polypeptide detection techniques described herein can then be cloned, expanded, and stored until these cells are used to produce the composition described here herein.

### Example 8. Using CRISPR/Cas to Knock Down a Transcript on an Endogenous Gene

This example illustrates a method of CRISPR/Cas such as or Cas13 knocking down expression level of an endogenous gene in mammary or mammary-like cells. In some embodiments, the endogenous gene can be any one of the gene described herein. In some embodiments, the endogenous gene can be any one of the casein described herein. In some embodiments, the endogenous gene can be an allergen described herein. In some cases, the endogenous gene can be a gene (e.g. LALBA) that is involved in lactose synthesis.

At least one vector encoding the components of the CRISPR/Cas13 (e.g. gRNA or Cas13) is introduced into the mammary or mammary-like cell. Cas13, guided by the gRNA, exerts RNase activity on the targeted transcript of the endogenous, thus knocking down the gene expression of the endogenous gene. The gene expression of the knocked down endogenous gene can be measured by any of the nucleic acid-based detection techniques described herein to detect mRNA of the knocked down endogenous gene or by polypeptide detection techniques to detect polypeptide encoded by the knocked out endogenous gene.

In some cases, the at least one vector encodes a deactivated version of Cas9 or Cas12 fused to an epigenetic modifier. The fusion of deactivated Cas and epigenetic modifier can knock down the gene expression of the endogenous gene by exerting epigenetic modification to the regulatory region of the endogenous gene.

In some embodiments, the CRISPR/Cas engineered mammary or mammary-like cells can be screened for the expression level of the knocked down endogenous gene, where the mammary or mammary-like cells can then be cloned and proliferated. For example, mammary or mammary-like cells comprising a knocked down endogenous gene of an allergen can be screened for the expression of the allergen. The mammary or mammary-like cells exhibiting suppressed or absent expression of the allergen as determined by the nucleic acid detection or the polypeptide detection techniques described herein can then be cloned, expanded, and stored until these cells are used to produce the composition described here herein.

### Example 9. Inactivated Cas to Modulate Gene Expression

This example illustrates methods for CRISPR-Cas engineering of mammary or mammary-like cells as described in the present disclosure for modulating gene expression. In some cases, the Cas can be a deactivated Cas, where the nuclease activity of the Cas is deactivated or removed. The deactivated Cas such as a deactivated Cas9 can be fused to an effector, where the gRNA complexing with the deactivated Cas can then direct the effector to a targeted region of the chromosome to modulate gene expression. In some embodiments, the targeted region can be a regulatory region described herein. In some cases, the targeted region can be nucleic acid sequence encoding any one of the gene described herein. In some instances, the effector can be a transcription activator (e.g. VP16, VP64, or p65.) In some embodiments, the effector can be transcription repressor, including KRAB, EnR, SID, etc.

In some embodiments, the effector can be an epigenetic modifier to remodel the epigenome that mediates the expression of the selected genes of interest. In some cases, epigenetic modifier can include methyltransferase, demethylase, dismutase, an alkylating enzyme, depurinase, oxidase, photolyase, integrase, transposase, recombinase, polymerase, ligase, helicase, glycosylase, acetyltransferase, deacetylase, kinase, phosphatase, ubiquitin-activating enzymes, ubiquitin-conjugating enzymes, ubiquitin ligase, deubiquitinating enzyme, adenylate-forming enzyme, AMPylator, de-AMPylator, SUMOylating enzyme, de SUMOylating enzyme, ribosylase, deribosylase, N-myristoyltransferase, chromotine remodeling enzyme, protease, oxidoreductase, transferase, hydrolase, lyase, isomerase, synthase, synthetase, or demyristoylation enzyme. In some instances, the epigenetic modifier is selected from a group consisting of p300, TET1, LSD1, HDAC1, HDAC8, HDAC4, HDAC11, HDT1, SIRT3, HST2, CobB, SIRT5, SIR2A, SIRT6, NUE, vSET, SUV39H1, DIMS, KYP, SUVR4, Set4, Setl, SETD8, and TgSET8. Epigenetic modification can be performed to increase expression of the selected genes of interest such as casein or α-lactalbumin. In some cases, the epigenetic remodeling can be performed to decrease expression of the selected genes that have been identified as allergens (e.g. casein).

### Example 10. Mammary or Mammary-Like Cells Expressing CRISPR/Cas

This example illustrates methods of generating mammary or mammary-like cells exhibiting Cas activity as described in the present disclosure. In some cases, the Cas described herein can be provided in any form. For example, a Cas protein can be provided in the form of a protein, such as a Cas protein alone or complexed with a guide nucleic acid. A Cas protein can be provided in the form of a nucleic acid encoding the Cas protein. In some embodiments, nucleic acid encoding Cas protein can be stably integrated in the genome of the mammary or mammary-like cell. In some cases, the nucleic acid encoding Cas protein can be a construct that is not integrated into the genome of the mammary or mammary-like cell. Nucleic acid encoding Cas protein can be operably linked to a promoter active in the mammary or mammary-like cell. Nucleic acid encoding Cas protein can be operably linked to a promoter in an expression construct. In some embodiments, the Cas protein is a dead Cas protein.

In some embodiments, the mammary or mammary-like cell described herein can be engineered to express Cas activity, where genetic modification of these cells can be induced by subsequent introduction of gRNA targeting the regulatory region or any one of gene described herein. In such arraignment, the mammary or mammary-like cell engineered to express Cas activity can be perpetually stored and expanded prior to the induction of the genetic modification.

Each range of values recited herein includes all combinations and sub-combinations of ranges, as well as specific numerals contained therein. All publications and patent applications cited in this specification are herein incorporated by reference to the extent not inconsistent with the description herein and for all purposes as if each individual publication or patent application were specifically and individually indicated to be incorporated by reference for all purposes.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof.

The herein described components (e.g., steps), devices, and objects and the description accompanying them are used as examples for the sake of conceptual clarity and that various configuration modifications using the disclosure provided herein are within the ordinary skill of those in the art. Consequently, as used herein, the specific examples set forth and the accompanying description are intended to be representative of their more general classes. In general, use of any specific example herein is also intended to be representative of its class, and the non-inclusion of such specific components (e.g., steps), devices, and objects herein should not be taken as indicating that limitation is desired.

With respect to the use of substantially any plural or singular terms herein, the reader can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely examples, and that, in fact, many other architectures can be implemented that achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components.

While particular aspects of the present subject matter described herein have been shown and described, it will be apparent to those in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is solely defined by the appended claims. In general, terms used herein and especially in the appended claims (e.g., bodies of the appended claims) are generally intended as “open” terms (e.g., the terms “include,” “includes,” or “including” may be interpreted as “including but not limited to,” the term “having” may be interpreted as “having at least”). If a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases may not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” may be interpreted to mean “at least one” or “one or more”); the same holds true for the use of definite articles used to introduce claim recitations. In addition, even if a specific number of an introduced claim recitation is explicitly recited, those skilled in the art will recognize that such recitation may be interpreted to mean at least the recited number (e.g., the bare recitation of “two recitations,” without other modifiers, may mean at least two recitations, or two or more recitations). Furthermore, in those instances where a convention analogous to “at least one of A, B, and C, etc.” is used, one having skill in the art can understand the convention (e.g., “compositions having at least one of A, B, and C” can include but not be limited to, compositions that have A alone, B alone, C alone, A and B together, A and C together, B and C together, and/or A, B, and C together, etc.). In those instances where a convention analogous to “A, B, or C” is used, one having skill in the art can understand the convention (e.g., “a composition having A, B, or C” can include but not be limited to compositions that have A alone, B alone, C alone, A and B together, A and C together, B and C together, and/or A, B, and C together, etc.).

The herein described aspects depict different components contained within, or connected with, different other components. It is to be understood that such depicted liquid compositions are merely exemplary, and that in fact many other liquid compositions can be implemented that achieve the same or similar functionality. In a conceptual sense, any arrangement of components to achieve the same or similar functionality is effectively “associated” such that the desired functionality is achieved.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for generating an engineered cell, the method comprising:
(a) providing a non-mammary adult stem cell or derivative thereof and
(b) subjecting said non-mammary adult stem cell or derivative thereof to conditions sufficient to generate said engineered cell, wherein said engineered cell is capable of lactation.

2. The method of claim 1, wherein (b) is performed in vitro.

3. The method of claim 1, wherein said engineered cell is capable of producing a milk component.

4. The method of claim 1, wherein said non-mammary adult stem cell is a mesenchymal stem cell.

5. The method of claim 1, wherein (b) comprises contacting said non-mammary adult stem cell or derivative thereof with a heterologous polypeptide including a gene regulating moiety, wherein said gene regulating moiety is configured to regulate an expression of a target gene, a target transcript, or a target protein of said non-mammary adult stem cell or derivative thereof, thereby effecting a modified expression profile of said target gene, said target transcript, or said target protein in said engineered cell as compared to a corresponding non-engineered non-mammary adult stem cell.

6. The method of claim 5, wherein said target gene, said target transcript, or said target protein is associated with production of a milk component selected from the group consisting of a milk protein, a milk carbohydrate, a milk fat, a milk lipid, and a combination thereof.

7. The method of claim 5, wherein said modified expression profile comprises an up-regulated expression of a gene or a transcript that degrades lactose.

8. The method of claim 5, wherein said modified expression profile is characteristic of a mammary cell lineage.

9. The method of claim 8, wherein said target gene, said target transcript, or said target protein is selected from the group consisting of: EpCAM, CD14, CD24, Muc1, CD29, CD49b, CD49f, CD61, Sca1, Prominin, ALDEFLUOR, CK14, CK18, and a combination thereof.

10. The method of claim 5, wherein said gene regulating moiety is an endonuclease.

11. The method of claim 10, wherein said endonuclease is a clustered regularly interspaced short palindromic repeats (CRISPR)-associated (Cas) endonuclease.

12. The method of claim 1, wherein said engineered cell is configured to generate a milk component in response to a hormone or a signaling factor.

13. A composition, comprising an engineered non-mammary adult stem cell exhibiting a modified expression profile that is different than an expression profile of a corresponding non-engineered non-mammary adult stem cell, wherein said engineered non-mammary adult stem cell is configured to be differentiated to an engineered cell capable of lactation.

14. The composition of claim 13, wherein said engineered non-mammary adult stem cell is configured to produce a milk component.

15. The composition of claim 13, wherein said engineered non-mammary adult stem cell is an engineered mesenchymal stem cell.

16. The composition of claim 15, wherein said corresponding non-engineered non-mammary adult stem cell is a corresponding non-engineered mesenchymal stem cell.

17. The composition of claim 13, wherein said engineered non-mammary adult stem cell or an ancestor cell thereof has been brought in contact with a heterologous polypeptide, which heterologous polypeptide includes a gene regulating moiety.

18. The composition of claim 17, wherein said gene regulating moiety is a polynucleotide-guided gene regulating moiety.

19. The composition of claim 17, wherein said gene regulating moiety is an endonuclease.

20. The composition of claim 19, wherein said endonuclease is a clustered regularly interspaced short palindromic repeats (CRISPR)-associated (Cas) endonuclease.

21. The composition of claim 17, wherein said gene regulating moiety is configured to regulate an expression of a target gene, a target transcript, or a target protein of said engineered non-mammary adult stem cell.

22. The composition of claim 21, wherein said target gene, said target transcript, or said target protein is associated with production of a milk component.

23. The composition of claim 22, wherein said milk component is a milk protein, a milk carbohydrate, a milk fat, or a milk lipid.

24. The composition of claim 21, wherein said target gene, said target transcript, or said target protein is a gene or a transcript that regulates a cellular response to a hormone or a signaling factor.

25. The composition of claim 13, wherein said modified expression profile comprises an up-regulated expression of a gene or a transcript that degrades lactose.

26. The composition of claim 13, wherein said modified expression profile is characteristic of a mammary cell lineage.

27. The composition of claim 26, wherein said modified expression profile comprises a modified expression profile of a gene, a transcript, or a protein selected from the group consisting of EpCAM, CD14, CD24, Muc1, CD29, CD49b, CD49f, CD61, Sca1, Prominin, ALDEFLUOR, CK14, CK18, and a combination thereof.

28. A method for generating a milk component, the method comprising:
(a) providing a reaction vessel including a cell culture, which cell culture comprises an engineered mammary or mammary-like cell derived from a non-mammary adult stem cell; and
(b) subjecting said cell culture to conditions sufficient to produce said milk component in vitro.

29. The method of claim 28, wherein said milk component comprises whey protein, casein, a lipid, an oligosaccharide, or a combination thereof.

30. The method of claim 28, wherein said engineered mammary or mammary-like cell is an engineered milk-producing cell.
